(19) United States
(12) Patent Application Publication
(10) Pub. No.: US 2024/0254659 A1
(43) Pub. Date: Aug. 1, 2024

(54) SYSTEMS AND METHODS FOR REGULATING TARGET GENES

(71) Applicant: Epicrispr Biotechnologies, Inc., South San Francisco, CA (US)

(72) Inventors: Daniel O. Hart, Oakland, CA (US); Lei S. Qi, Palo Alto, CA (US); Timothy Daley, San Francisco, CA (US); Thomas Blair Gainous, Oakland, CA (US); Giovanni Carosso, San Francisco, CA (US); Tengyu Ko, Redwood City, CA (US); Robin W. Yeo, San Francisco, CA (US); Christopher Darryl Still, II, South San Francisco, CA (US)

(21) Appl. No.: 18/417,827

(22) Filed: Jan. 19, 2024

Related U.S. Application Data
(63) Continuation of application No. PCT/US2022/073920, filed on Jul. 20, 2022.
(60) Provisional application No. 63/223,842, filed on Jul. 20, 2021.

(51) Int. Cl. C40B 30/06; C12N 9/22; C12N 15/113

(52) U.S. Cl. CPC C40B 30/06; C12N 9/22; C12N 15/113; C12N 2310/20

(57) ABSTRACT

The disclosure provides compositions, methods, and systems for modulating expression of target genes (e.g., target endogenous genes). Complexes of the disclosure can be useful for bringing one or more heterologous gene effectors into close proximity with a target gene or target gene regulatory sequence, thereby facilitating modulation of an expression or activity level of the target gene. The disclosure provides, for example, compositions, systems, and methods for high throughput screens to identify heterologous gene effector domains and complexes of the effector domains with guide moieties to modulate expression of particular target gene(s). The disclosure also provides complexes identified by systems disclosed herein that can be employed for other purposes, such as research or as therapeutics.

### CROSS REFERENCE

This application is a continuation application of International Patent Application No. PCT/US2022/073920, filed Jul. 20, 2022, which claims the benefit of U.S. Provisional Application No. 63/223,842, filed Jul. 20, 2021, each of which is incorporated herein by reference in its entirety.

### SEQUENCE LISTING

The instant application is being filed along with a Sequence Listing in electronic format. The Sequence Listing is provided as a file entitled 55176-715_301_SL.xml and is 60,526,592 bytes in size. The information in the electronic format of the Sequence Listing is herein incorporated by reference in its entirety.

### BACKGROUND

Complex networks regulate gene expression, underpinning survival, growth, differentiation, and various physiological functions of cells. In some cases, one or more specific genes can be turned on or turned off to effect such regulation in the cells. In some cases, aberrant expression of particular genes contributes to many diseases and conditions. Aberrant expression of a gene of interest can be abnormally increased expression level of the gene, abnormally decreased expression level of the gene, abnormally prolonged duration of expression of the gene, or abnormally shortened duration of expression of the gene.

### SUMMARY

Agents that are capable of modulating expression of specific genes in a desirable way can have therapeutic benefit, but many strategies that are currently employed fail to elicit effects that are robust, persistent, and/or reversible. In addition, only a selected few gene effectors have been explored and utilized to regulate a wide variety of target genes. Thus, various aspects of the present disclosure provide systems, methods, and compositions comprising one or more gene effectors that can be tailor-made for regulating a specific target gene (e.g., upregulating expression, downregulating expression, prolonging or shortening duration of expression, etc.).

Disclosed herein, in some aspects, is a method, comprising: (a) contacting a population of cells with a library of complexes, wherein an individual complex of the library comprises: (i) a heterologous gene effector that is different from heterologous gene effectors in other complexes of the library; and (ii) a guide nucleic acid sequence that exhibits 100% sequence identity to guide nucleic acid sequences in the other complexes of the library, wherein the heterologous gene effector and the guide nucleic acid molecule form the individual complex that exhibits specific binding to a target endogenous gene in the population of cells, and wherein the library comprises at least 25 different complexes; (b) upon the contacting, sorting the population of cells based on a change in expression or activity level of the target endogenous gene in the population of cells; and (c) identifying one or more lead heterologous gene effectors of the library that effect the change.

Disclosed herein, in some aspects, is a method, comprising: (a) contacting a population of cells with a library of complexes, wherein an individual complex of the library comprises: (i) a heterologous gene effector that is different from heterologous gene effectors in other complexes of the library; and (ii) a guide nucleic acid sequence that exhibits 100% sequence identity to guide nucleic acid sequences in the other complexes of the library, wherein the heterologous gene effector and the guide nucleic acid sequence form the individual complex that exhibits specific binding to a target endogenous gene in the population of cells, and wherein the heterologous gene effector comprises a viral gene effector; (b) upon the contacting, sorting the population of cells based on a change in expression or activity level of the target endogenous gene in the population of cells; and (c) identifying one or more lead heterologous gene effectors of the library that effect the change.

In some embodiments, the viral gene effector is derived from a human virus selected from the group consisting of Adenoviridae, Arenaviridae, Bornaviridae, Coronaviridae, Filoviridae, Flaviviridae, Hepadnaviridae, Herpesviridae, Orthomyxoviridae, Papillomaviridae, Paramyxoviridae, Parvoviridae, Peribunyaviridae, Phenuiviridae, Pneumoviridae, Polyomaviridae, Poxviridae, Retroviridae, and Rhabdoviridae. In some embodiments, the viral gene effector is derived from a human-bat shared virus selected from the group consisting of Flaviviridae, Lyssaviridae, Filoviridae, Paramyxoviridae, Orthomyxoviridae, Coronaviridae, Reoviridae, Togaviridae, Phenuviridae, and Hantaviridae. In some embodiments, the viral gene effector is derived from a virus selected from the group consisting of Archaea-tropic virus, Siphoviridae, podoviridae, Mimiviridae, Nimaviridae, Ligamenvirales, Globuloviridae, Fuselloviridae, Bicaudaviridae, Satellite virus, Iridoviridae, Turriviridae, Caudovirales, Phycodnaviridae and Myoviridae. In some embodiments, the library comprises at least 30, 50, 100, 200, 500, 1,000, 2,000, 5,000, or 10,000 different complexes. In some embodiments, the guide nucleic acid sequence comprises between about 10 and about 30 nucleotides, between about 15 and about 25 nucleotides, or about 15 nucleotides. In some embodiments, the individual complex further comprises a heterologous endonuclease. In some embodiments, the heterologous endonuclease of the individual complex exhibits 100% sequence identity to heterologous endonucleases of the other complexes. In some embodiments, the heterologous gene effector and the heterologous endonuclease are fused to each other. In some embodiments, the heterologous gene effector and the heterologous endonuclease are non-covalently coupled to each other. In some embodiments, the heterologous endonuclease is a Cas protein. In some embodiments, the Cas protein lacks nucleic acid cleavage activity. In some embodiments, the guide nucleic acid sequence is a part of a guide RNA molecule. In some embodiments, the heterologous gene effector comprises a heterologous transcriptional regulator. In some embodiments, the heterologous gene effector comprises a heterologous chromatin regulator. In some embodiments, the change is enhanced expression or activity level of the target endogenous gene. In some embodiments, the change is reduced expression or activity level of the target endogenous gene. In some embodiments, the heterologous gene effector exhibits at least about 80%, at least about 85%, at least about 90%, at least about 95%, at least about 99%, or 100% sequence identity to any one of SEQ ID NOs: 16-16154 or any one of SEQ ID NOs: 16-13605. In some embodiments, the heterologous gene effector exhibits at least about 80%, at least about 85%, at least about 90%, at least about 95%, at least about 99%, or 100% sequence identity to any one of SEQ ID NOs: 16155-47350 or any one of SEQ ID NOs: 16155-43953. In some embodiments, the heterologous gene effector comprises a plurality of different heterologous gene effectors, wherein a combination of the plurality of different heterologous gene effectors of the individual complex is different from combinations of heterologous gene effectors within other complexes of the library. In some embodiments, the plurality of different heterologous gene effectors is not P300, TET1, TET2, TET3, and/or HSF1. In some embodiments, at least one of the plurality of different heterologous gene effectors is not VP64, P65, Rta, VPR, AD2, CR3, ELKF1, GATA4, PRVIE, p53, SP1, MYOD, MEF2C, TAX, PPAR-gamma, MED1, MED7, MED17, MED26, MED29, TBP, GTF2H-2D, GTF2B, CBP, HSF1, MS2-p65-HSF1, MS2-TET1, NLS-dCas9-VP64, P300, p65, PRDM9, PUFa-GADD45A-TET1, R2, SunTag-scFv-sfGFP-TET1CD, TET1, TET2, TET3, VP120, VP16, VP16, VP16, VP48, VP64, VP64 or p65+/−HSF1 or MyoD1, and/or VPR (Vp64+p65+Rta). In some embodiments, at least one of the plurality of different heterologous gene effectors is not KRAB, Mad mSIN3 interaction domain (SID), ERF repressor domain (ERD), cat3a, last 301 amino acids of Dnmt3a Isoform 1, dCas9-KRAB-MeCP2, DNMT3A, DNMT3A, DNMT3A, DNMT3A R887E-DNMT3L, DNMT3A-DNMT3L, DNMT3B, EZH2, HDAC, KRAB-DNMT3A, KRAB-DNMT3A-DNMT3L, KRAB-DNMT3L, LSD1, M.SssI, MQ1, MQ1 Q147E, SID4×, and/or SuntTag-DNMT3A. In some embodiments, the heterologous gene effector is not P300, TET1, TET2, TET3, and/or HSF1. In some embodiments, the heterologous gene effector is not VP64, P65, Rta, VPR, AD2, CR3, ELKF1, GATA4, PRVIE, p53, SP1, MYOD, MEF2C, TAX, PPAR-gamma, MED1, MED7, MED17, MED26, MED29, TBP, GTF2H-2D, GTF2B, CBP, HSF1, MS2-p65-HSF1, MS2-TET1, NLS-dCas9-VP64, P300, p65, PRDM9, PUFa-GADD45A-TET1, R2, SunTag-scFv-sfGFP-TET1CD, TET1, TET2, TET3, VP120, VP16, VP16, VP16, VP48, VP64, VP64 or p65+/−HSF1 or MyoD1, and/or VPR (Vp64+p65+Rta). In some embodiments, the heterologous gene effector is not KRAB, Mad mSIN3 interaction domain (SID), ERF repressor domain (ERD), cat3a, last 301 amino acids of Dnmt3a Isoform 1, dCas9-KRAB-MeCP2, DNMT3A, DNMT3A, DNMT3A, DNMT3A R887E-DNMT3L, DNMT3A-DNMT3L, DNMT3B, EZH2, HDAC, KRAB-DNMT3A, KRAB-DNMT3A-DNMT3L, KRAB-DNMT3L, LSD1, M.SssI, MQ1, MQ1 Q147E, SID4×, and/or SuntTag-DNMT3A. In some embodiments, the one or more lead heterologous gene effectors is at most 1 heterologous gene effector, at most 2 heterologous gene effectors, at most 5 heterologous gene effectors, at most 10 heterologous gene effectors, at most 15 heterologous gene effectors, at most 20 heterologous gene effectors, or at most 50 heterologous gene effectors. In some embodiments, a degree of the change in the expression or the activity level of the target endogenous gene effected by the one or more lead heterologous gene effectors is greater than that by a control by at least 2-fold. In some embodiments, the degree is greater than that by the control by at least 5-fold, 10-fold, 20-fold, 50-fold, or 100-fold. In some embodiments, the control is a population of cells without the library of complexes. In some embodiments, the control is a population of cells contacted by a control heterologous gene effector. In some embodiments, the control heterologous gene effector is P300, TET1, TET2, TET3, and/or HSF1. In some embodiments, the control heterologous gene effector is VP64, P65, Rta, VPR, AD2, CR3, ELKF1, GATA4, PRVIE, p53, SP1, MYOD, MEF2C, TAX, PPAR-gamma, MED1, MED7, MED17, MED26, MED29, TBP, GTF2H-2D, GTF2B, CBP, HSF1, MS2-p65-HSF1, MS2-TET1, NLS-dCas9-VP64, P300, p65, PRDM9, PUFa-GADD45A-TET1, R2, SunTag-scFv-sfGFP-TET1CD, TET1, TET2, TET3, VP120, VP16, VP16, VP16, VP48, VP64, VP64 or p65+/−HSF1 or MyoD1, and/or VPR (Vp64+p65+Rta). In some embodiments, the control heterologous gene effector is KRAB, SID, ERD, cat3a, last 301 amino acids of Dnmt3a Isoform 1, dCas9-KRAB-MeCP2, DNMT3A, DNMT3A, DNMT3A, DNMT3A R887E-DNMT3L, DNMT3A-DNMT3L, DNMT3B, EZH2, HDAC, KRAB-DNMT3A, KRAB-DNMT3A-DNMT3L, KRAB-DNMT3L, LSD1, M.SssI, MQ1, MQ1 Q147E, SID4×, and/or SuntTag-DNMT3A. In some embodiments, the method further comprises performing (a)-(c) for an additional target endogenous gene in an additional population of cells, wherein (1) the one or more lead heterologous gene effectors of the library that effects the change in expression or activity level of the target endogenous gene is different from (2) one or more lead heterologous gene effectors of the library that effects a change in expression or activity level of the additional target endogenous gene. In some embodiments, the population of cells and the additional population of cells are of the same cell types. In some embodiments, the population of cells and the additional population of cells are of different cell types. In some embodiments, the population of cells comprises mammalian cells. In some embodiments, the population of cells comprises human cells. In some embodiments, the population of cells comprises stem cells. In some embodiments, the population of cells comprises differentiated cells. In some embodiments, the target endogenous gene is a disease-associated gene. In some embodiments, the target endogenous gene is a differentiation-associated gene. In some embodiments, the target endogenous gene is an age-related gene.

Disclosed herein, in some aspects, is a system comprising the library of complexes of the method of any one of preceding embodiments.

Disclosed herein, in some aspects, is a kit comprising the library of complexes of the method of any one of preceding embodiments.

Disclosed herein, in some aspects, is a nucleic acid library encoding the heterologous gene effectors of the library of complexes of the method of any one of preceding embodiments.

Disclosed herein, in some aspects, is a nucleic acid library encoding the library of complexes of the method of any one of preceding embodiments.

Disclosed herein, in some aspects, is a population of cells collectively expressing the heterologous gene effectors of the library of complexes of the method of any one of preceding embodiments.

Disclosed herein, in some aspects, is a population of cells collectively expressing the library of complexes of the method of any one of preceding embodiments.

Disclosed herein, in some aspects, is a complex comprising a guide moiety and a heterologous gene effector, wherein the heterologous gene effector comprises an amino acid sequence with at least about 70% sequence identity to any one of SEQ ID NOs: 23631, 1102, 2057, 5543, 9066, 11948, 15646, 17629, 19860, 21015, 21166, 22149, 22707, 23639, 25430, 25555, 32678, 33890, 34047, 35737, 38138, 38780, 40913, 40985, 40986, and 42623.

In some embodiments, the amino acid sequence has at least 90% sequence identity to any one of SEQ ID NOs: 23631, 1102, 2057, 5543, 9066, 11948, 15646, 17629, 19860, 21015, 21166, 22149, 22707, 23639, 25430, 25555, 32678, 33890, 34047, 35737, 38138, 38780, 40913, 40985, 40986, and 42623. In some embodiments, the heterologous gene effector comprises the amino acid sequence of any one of SEQ ID NOs: 23631, 1102, 2057, 5543, 9066, 11948, 15646, 17629, 19860, 21015, 21166, 22149, 22707, 23639, 25430, 25555, 32678, 33890, 34047, 35737, 38138, 38780, 40913, 40985, 40986, and 42623. In some embodiments, the amino acid sequence has at least 90% sequence identity to SEQ ID NO: 23631. In some embodiments, the heterologous gene effector comprises the amino acid sequence of SEQ ID NO: 23631. In some embodiments, the amino acid sequence has at least 90% sequence identity to SEQ ID NO: 33890. In some embodiments, the heterologous gene effector comprises the amino acid sequence of SEQ ID NO: 33890. In some embodiments, the amino acid sequence has at least 90% sequence identity to SEQ ID NO: 40985. In some embodiments, the heterologous gene effector comprises the amino acid sequence of SEQ ID NO: 40985. In some embodiments, the heterologous gene effector contains less than 500 amino acids. In some embodiments, the heterologous gene effector contains less than 100 amino acids. In some embodiments, the guide moiety specifically binds to a target gene or a target gene regulatory sequence. In some embodiments, the guide moiety comprises a guide nucleic acid sequence. In some embodiments, the guide nucleic acid sequence comprises or consists of between about 10 and about 30 nucleotides. In some embodiments, the guide nucleic acid sequence is a guide RNA. In some embodiments, the guide nucleic acid sequence is a single guide RNA (sgRNA). In some embodiments, the guide moiety comprises a nuclease or a part thereof. In some embodiments, the nuclease or part thereof is a modified nuclease that has reduced nuclease activity compared to a wild-type version of the nuclease. In some embodiments, the nuclease or part thereof substantially lacks nucleic acid cleavage activity. In some embodiments, the nuclease or part thereof is a Cas protein or part thereof. In some embodiments, the nuclease or part thereof is a nuclease deactivated Cas (dCas) protein or part thereof. In some embodiments, the guide moiety and the heterologous gene effector are fused to each other, optionally via a linker. In some embodiments, the guide moiety and the heterologous gene effector are non-covalently coupled to each other.

Disclosed herein, in some aspects, is a vector comprising the heterologous gene effector of any one of the preceding embodiments. In some embodiments, the vector further comprises the guide moiety.

Disclosed herein, in some aspects, is a vector comprising the complex of any one of the preceding embodiments.

Disclosed herein, in some aspects, is a vector comprising a nucleic acid that encodes the heterologous gene effector of any one of the preceding embodiments.

In some embodiments, the vector further comprises a nucleic acid that encodes the guide moiety or a component thereof. In some embodiments, the vector is a viral vector. In some embodiments, the vector is a non-viral vector.

Disclosed herein, in some aspects, is a population of cells comprising the complex of any one of the preceding embodiments.

Disclosed herein, in some aspects, is a method of modulating expression or activity of a target gene, the method comprising contacting a population of cells that comprise the target gene with the complex or the vector of any one of the preceding embodiments.

In some embodiments, the population of cells comprises mammalian cells. In some embodiments, the population of cells comprises human cells. In some embodiments, the population of cells comprises stem cells. In some embodiments, the population of cells comprises differentiated cells. In some embodiments, the contacting is in vitro or ex vivo. In some embodiments, the contacting is in vivo.

Disclosed herein, in some aspects, is a method of treating a subject in need thereof, the method comprising administering to the subject the complex or vector of any one of the preceding embodiments, thereby modulating expression or activity of a target gene in a population of cells in the subject.

In some embodiments, the target gene is a target endogenous gene. In some embodiments, the target gene is a disease-associated gene. In some embodiments, the target gene is a differentiation-associated gene. In some embodiments, the modulating expression or activity comprises increasing expression or activity level of the target gene. In some embodiments, the modulating expression or activity comprises reducing expression or activity level of the target gene. In some embodiments, expression or activity of the target gene is increased at least 2-fold compared to a control. In some embodiments, expression or activity of the target gene is reduced at least 2-fold compared to a control. In some embodiments, the expression or activity of the target gene is modulated for a period of time that is at least 10% longer than a control. In some embodiments, the expression or activity of the target gene is modulated for at least 12 hours. In some embodiments, the expression or activity of the target gene is modulated for at least 28 days. In some embodiments, the control is the population of cells prior to the contacting. In some embodiments, the control is a population of cells not contacted with the complex. In some embodiments, the control is a population of cells contacted with a control complex.

Disclosed herein, in some aspects, is an expression vector comprising: a plurality of heterologous polynucleotide sequences, wherein each heterologous polynucleotide sequence of the plurality of heterologous polynucleotide sequences exhibits at least about 80% sequence identity to the polynucleotide sequence of any one or more of SEQ ID NOs: 49334-49341 or 49344-49352.

In some embodiments of any one of the expression vectors disclosed herein, each heterologous polynucleotide sequence exhibits at least about 80% sequence identity to SEQ ID NO: 49334. In some embodiments of any one of the expression vectors disclosed herein, each heterologous polynucleotide sequence exhibits at least about 80% sequence identity to SEQ ID NO: 49335. In some embodiments of any one of the expression vectors disclosed herein, each heterologous polynucleotide sequence exhibits at least about 80% sequence identity to SEQ ID NO: 49336. In some embodiments of any one of the expression vectors disclosed herein, each heterologous polynucleotide sequence exhibits at least about 80% sequence identity to SEQ ID NO: 49337. In some embodiments of any one of the expression vectors disclosed herein, each heterologous polynucleotide sequence exhibits at least about 80% sequence identity to SEQ ID NO: 49338. In some embodiments of any one of the expression vectors disclosed herein, each heterologous polynucleotide sequence exhibits at least about 80% sequence identity to SEQ ID NO: 49339. In some embodiments of any one of the expression vectors disclosed herein, each heterologous polynucleotide sequence exhibits at least about 80% sequence identity to SEQ ID NO: 49340. In some embodiments of any one of the expression vectors disclosed herein, each heterologous polynucleotide sequence exhibits at least about 80% sequence identity to SEQ ID NO: 49341. In some embodiments of any one of the expression vectors disclosed herein, each heterologous polynucleotide sequence exhibits at least about 80% sequence identity to SEQ ID NO: 49344. In some embodiments of any one of the expression vectors disclosed herein, each heterologous polynucleotide sequence exhibits at least about 80% sequence identity to SEQ ID NO: 49345. In some embodiments of any one of the expression vectors disclosed herein, each heterologous polynucleotide sequence exhibits at least about 80% sequence identity to SEQ ID NO: 49346. In some embodiments of any one of the expression vectors disclosed herein, each heterologous polynucleotide sequence exhibits at least about 80% sequence identity to SEQ ID NO: 49347. In some embodiments of any one of the expression vectors disclosed herein, each heterologous polynucleotide sequence exhibits at least about 80% sequence identity to SEQ ID NO: 49348. In some embodiments of any one of the expression vectors disclosed herein, each heterologous polynucleotide sequence exhibits at least about 80% sequence identity to SEQ ID NO: 49349. In some embodiments of any one of the expression vectors disclosed herein, each heterologous polynucleotide sequence exhibits at least about 80% sequence identity to SEQ ID NO: 49350. In some embodiments of any one of the expression vectors disclosed herein, each heterologous polynucleotide sequence exhibits at least about 80% sequence identity to SEQ ID NO: 49351. In some embodiments of any one of the expression vectors disclosed herein, each heterologous polynucleotide sequence exhibits at least about 80% sequence identity to SEQ ID NO: 49352.

In some embodiments of any one of the expression vectors disclosed herein, each heterologous polynucleotide sequence exhibits at least about 90% sequence identity to the polynucleotide sequence of any one of SEQ ID NOs: 49334-49341 or 49344-49352. In some embodiments of any one of the expression vectors disclosed herein, each heterologous polynucleotide sequence exhibits at least about 95% sequence identity to the polynucleotide sequence of any one of SEQ ID NOs: 49334-49341 or 49344-49352.

In some embodiments of any one of the expression vectors disclosed herein, each heterologous polynucleotide sequence comprises (i) a CRISPR target sequence and (ii) one or more CRISPR protospacer adjacent motif (PAM) sequences. In some embodiments of any one of the expression vectors disclosed herein, the CRISPR target sequence is flanked by two different CRISPR PAM sequences. In some embodiments of any one of the expression vectors disclosed herein, the one or more CRISPR PAM sequences comprises a Cas12 PAM sequence. In some embodiments of any one of the expression vectors disclosed herein, the one or more CRISPR PAM sequences comprises a Cas9 PAM sequence.

In some embodiments of any one of the expression vectors disclosed herein, the plurality comprises 4 or more heterologous polynucleotide sequences. In some embodiments of any one of the expression vectors disclosed herein, the plurality comprises 6 or more heterologous polynucleotide sequences.

In another aspect, the present disclosure provides a nucleic acid molecule comprising: a heterologous polynucleotide sequence that is a chimeric sequence comprising (i) a CRISPR target sequence and (ii) a CRISPR protospacer adjacent motif (PAM) sequence and an additional CRISPR PAM sequence that are different, wherein the CRISPR target sequence is flanked by the CRISPR PAM sequence and the additional CRISPR PAM sequence.

In some embodiments of any one of the nucleic acid molecules disclosed herein, the heterologous polynucleotide sequence is a single strand.

In some embodiments of any one of the nucleic acid molecules disclosed herein, a distance between the CRISPR PAM sequence and the additional CRISPR PAM sequence is at most about 50 nucleobases. In some embodiments of any one of the nucleic acid molecules disclosed herein, the distance is at most about 40 nucleobases. In some embodiments of any one of the nucleic acid molecules disclosed herein, the distance is at most about 35 nucleobases. In some embodiments of any one of the nucleic acid molecules disclosed herein, the distance is at most about 25 nucleobases. In some embodiments of any one of the nucleic acid molecules disclosed herein, the distance is at most about 20 nucleobases.

In some embodiments of any one of the nucleic acid molecule disclosed herein, a size of the CRISPR target sequence is at least about 10 nucleobases. In some embodiments of any one of the nucleic acid molecules disclosed herein, the size is at least about 15 nucleobases.

In some embodiments of any one of the nucleic acid molecules disclosed herein, the CRISPR PAM sequence and the additional CRISPR PAM sequence are recognized by different CRISPR types. In some embodiments of any one of the nucleic acid molecules disclosed herein, one of the CRISPR PAM sequence and the additional CRISPR PAM sequence is recognized by Cas12 or a variant thereof. In some embodiments of any one of the nucleic acid molecules disclosed herein, one of the CRISPR PAM sequence and the additional CRISPR PAM sequence is recognized by Cas9 or a variant thereof.

In some embodiments of any one of the nucleic acid molecule disclosed herein, the heterologous polynucleotide sequence is a non-coding sequence.

In some embodiments of any one of the nucleic acid molecules disclosed herein, the CRISPR target sequence exhibits at least about 80% sequence identity to SEQ ID NO: 49334.c

In another aspect, the present disclosure provides a nucleic acid molecule comprising: a plurality of heterologous polynucleotide sequences, wherein: (i) each heterologous polynucleotide sequence of the plurality of heterologous polynucleotide sequences comprises a polynucleotide sequence and an additional polynucleotide sequence that are derived from different human chromosomes; and (ii) a size of each heterologous polynucleotide sequence is at most about 50 nucleobases.

In some embodiments of any one of the nucleic acid molecules disclosed herein, the size is at most about 40 nucleobases. In some embodiments of any one of the nucleic acid molecules disclosed herein, the size is at most about 30 nucleobases. In some embodiments of any one of the nucleic acid molecules disclosed herein, the size is at most about 25 nucleobases.

In some embodiments of any one of the nucleic acid molecules disclosed herein, a size of the polynucleotide sequence is at least about 5 nucleobases. In some embodiments of any one of the nucleic acid molecules disclosed herein, a size of the additional polynucleotide sequence is at least about 5 nucleobases.

In some embodiments of any one of the nucleic acid molecules disclosed herein, a distance between a heterologous polynucleotide sequence and an additional heterologous polynucleotide sequence of the plurality is at most about 100 nucleobases.

In some embodiments of any one of the nucleic acid molecules disclosed herein, the distance is at most about 80 nucleobases. In some embodiments of any one of the nucleic acid molecules disclosed herein, the distance is at most about 60 nucleobases. In some embodiments of any one of the nucleic acid molecules disclosed herein, the distance is at most about 50 nucleobases.

In some embodiments of any one of the nucleic acid molecules disclosed herein, each of the heterologous polynucleotide sequences is a non-coding sequence.

In some embodiments of any one of the nucleic acid molecules disclosed herein, at least one of the polynucleotide sequence and the additional polynucleotide sequence is derived from a Cluster of Differentiation (CD) protein.

In some embodiments of any one of the nucleic acid molecules disclosed herein, the plurality comprises 4 or more heterologous polynucleotide sequences. In some embodiments of any one of the nucleic acid molecules disclosed herein, the plurality comprises 6 or more heterologous polynucleotide sequences.

In another aspect, the present disclosure provides an expression vector comprising any one of the nucleic acid sequences disclosed herein.

In some embodiments of any one of the expression vectors or the nucleic acid molecules disclosed herein, the expression vector or the nucleic acid molecule further comprises an Upstream Activating Sequence (UAS) that is downstream of the heterologous polynucleotide sequence or the heterologous polynucleotide sequences. In some embodiments of any one of the expression vectors or the nucleic acid molecules disclosed herein, the UAS is a non-human UAS. In some embodiments of any one of the expression vector or the nucleic acid molecule disclosed herein, the UAS is derived from yeast GAL4 promoter.

In some embodiments of any one of the expression vectors or the nucleic acid molecules disclosed herein, the expression vector or the nucleic acid molecule further comprises a promoter.

In some embodiments of any one of the expression vectors or the nucleic acid molecules disclosed herein, at least one of the plurality of heterologous polynucleotide sequences is upstream of the promoter. In some embodiments of any one of the expression vectors or the nucleic acid molecules disclosed herein, the promoter is a strong constitutive human promoter. In some embodiments of any one of the nucleic acid molecules disclosed herein, the promoter is a weak minimal viral promoter. In some embodiments of any one of the nucleic acid molecules disclosed herein, the expression vector or the nucleic acid molecule further comprises a target gene under the control of the promoter.

In another aspect, the present disclosure provides a cell comprising any one of the expression vectors or the nucleic acid sequences as disclosed herein. In some embodiments, the cell can be a mammalian cell.

In another aspect, the present disclosure provides a method of regulating expression of a target gene in a cell, the method comprising: (a) providing a vector comprising (i) any one of the nucleic acid sequences as disclosed herein, (ii) a promoter, and (ii) the target gene; and contacting the nucleic acid sequence with an actuator moiety capable of interacting with the promoter to modulate expression level of the target gene.

In some embodiments of any one of the methods disclosed herein, the actuator moiety is a complex comprising a CRISPR endonuclease and a gene effector. In some embodiments of any one of the methods disclosed herein, the gene effector is a gene activator. In some embodiments of any one of the methods disclosed herein, the gene effector is a gene repressor. In some embodiments of any one of the methods disclosed herein, the complex further comprises a guide nucleic acid molecule exhibiting specific binding to the heterologous polynucleotide sequence or the heterologous polynucleotide sequence.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 provides an overview of an illustrative effector screen design to identify human nuclear proteins that activate expression of CD45 or reduce expression of CD71.

FIG. 2 illustrates a starting vector backbone that can be used for generating a vector of the disclosure.

FIG. 3 illustrates a vector of the disclosure that encodes a dCas9 fused to a heterologous effector, with an IRES driving expression of a downstream reporter gene.

FIG. 4 shows relative baseline expression of CD45 in illustrative cell lines.

FIG. 5 shows relative baseline expression of CD71 in illustrative cell lines.

FIG. 6 shows modulation of CD45 and CD71 by complexes of the disclosure, and cell sorting based on the resulting expression profiles of CD45 and CD71. Increased expression of CD45 can be observed in experimental conditions in the activator screen (top right panel), and reduced expression of CD71 can be observed in experimental conditions in the repressor screen (bottom right panel).

FIG. 7 provides an illustrative schematic of an expression construct for a combinatorial screen of the disclosure.

FIG. 8 provides an illustrative schematic of an expression construct for the generation of cell lines stably expressing GAI-dCas9-ABI. This reagent can be used in a combinatorial screen of the disclosure.

FIG. 9 illustrates modulation of CD45 and CD71 expression by complexes of the disclosure that comprise combinations of a transcriptional regulator and a chromatin regulator associated with dCas9. The top images show an illustrative activator screen for CD45, and the bottom images show an illustrative repressor screen for CD71. The graphs on the right illustrate an increase in CD45 expression by a complex of the disclosure, and repression of CD71 expression by a complex of the disclosure.

FIG. 10 schematically shows an example structure of an expression vector for an engineered synthetic reporter (ESR). FIG. 10 discloses SEQ ID NO: 49340 (full sequence shown), which includes SEQ ID NO: 49334 (“synthetic guide target”) SEQ ID NO: 49341 (“UAS”), SEQ ID NO: 49335 (Cas12a/MINI PAM+synthetic guide target), SEQ ID NO: 49336 (synthetic guide target+SpCas9 PAM), SEQ ID NO: 49337 (synthetic guide target+SpCas9 PAM and SaCas9 PAM), SEQ ID NO: 49338 (Cas12a/mini PAM+synthetic guide target+SpCas9 PAM and SaCas9 PAM), and SEQ ID NO: 49339 (Cas12a/mini PAM+synthetic guide target+SpCas9 PAM).

FIG. 11 schematically shows an example sequence of the ESR. FIG. 11 discloses SEQ ID NO: 49344 (single ESR repeat, which includes a Cas12a/mini PAM (TTTA), heterologous guide target (SEQ ID NO: 49334), Cas9 PAM (CGG), and UAS (SEQ ID NO: 49341)); SEQ ID NO: 49345 (7× copies of the ESR repeat); SEQ ID NO: 49346 (miniCMV promoter); and SEQ ID NO: 49347 (EF 1a promoter).

FIG. 12 schematically shows an example sequence of a control reporter vector. FIG. 12 discloses SEQ ID NO: 49348 (single TRE3G repeat, which includes a Cas12a/mini PAM (TTTA), a Cas9 PAM (CCC), a Cas9 spacer (SEQ ID NO: 49349), and a CasMINI/Cas12a spacer (SEQ ID NO: 49350)); and a full TRE3GS_promoter (SEQ ID NO: 49351, which includes 7 copies of the TRE3G repeat and a modified miniCMV promoter (SEQ ID NO: 49352)).

FIG. 13 shows flow cytometry data analysis of a reporter 293T cell line engineered with the ESR encoding miniCMV-GFP (ESR121).

FIG. 14 shows flow cytometry data analysis of a reporter 293T cell line engineered with the ESR encoding EF1a-GFP (ESR221).

FIG. 15 shows flow cytometry data analysis of a reporter K562 cell line engineered with the ESR encoding miniCMV-GFP (ESR111).

FIG. 16 shows flow cytometry data analysis of a reporter K562 cell line engineered with the ESR encoding EF1a-GFP (ESR211).

FIG. 17 shows a different flow cytometry data analysis of a reporter 293T cell line engineered with the ESR encoding miniCMV-GFP (ESR121).

FIG. 18 shows a different flow cytometry data analysis of a reporter 293T cell line engineered with the ESR encoding EF1a-GFP (ESR221).

FIG. 19 shows flow cytometry data analysis of a reporter 293T cell lines engineered with the ESR encoding green fluorescent protein (GFP) (clone 1), an additional reporter 293T cell lines engineered with the ESR encoding GFP (clone 2), a control reporter 293T cell line expressing a control expression vector encoding GFP (TRE3G-GFP), and an additional control reporter 293T cell line expressing a control expression vector encoding GFP (SV40-GFP), wherein ESR-EF1a 293T clonal cell lines show a narrower distribution of GFP expression than existing reporter cells.

FIG. 20 provides an overview of an illustrative screen design to identify heterologous gene effectors that activate or reduce expression of target genes (e.g., CD45, CD71), or a GFP reporter gene.

FIG. 21A provides representative flow cytometry histograms showing high dynamic range of ESR-GFP transcriptional activation or suppression from positive control constructs VPR or KRAB, respectively.

FIG. 21B provides representative histograms showing transcriptional activation or repression of two endogenous human gene targets: lowly-expressed CD45 for activation or highly-expressed CD71 for suppression.

FIG. 22A is a volcano plot showing hits in a screen for candidate activator heterologous gene effectors using an enhanced synthetic reporter system.

FIG. 22B is a volcano plot showing hits in a screen for candidate repressor heterologous gene effectors using an enhanced synthetic reporter system.

FIG. 23A is a volcano plot showing hits in a screen for candidate activator heterologous gene effectors using an endogenous gene (CD45) in wild type K562 cells.

FIG. 23B is a volcano plot showing hits in a screen for candidate repressor heterologous gene effectors using an endogenous gene (CD71) in wild type K562 cells.

FIG. 24A shows the geometric mean of CXCR4 expression 3 and 7 days after transfection with plasmids encoding complexes that comprise heterologous gene effectors disclosed herein targeted to CXCR4 by a sgRNA.

FIG. 24B provides representative flow cytometry histograms of CXCR4-APC fluorescence at 3 d.p.t., showing relative performance of one novel activator (EPICXV.1) versus canonical activator (VPR), and one novel suppressor (EPICXV.71) versus canonical suppressor (KRAB). Modulators shown are fused to dCasMini.

FIG. 24C illustrates relative sizes (bp) of sequences encoding dCas9, dCasMini, canonical activator VPR, and fusions thereof, as compared to the novel candidate effectors disclosed herein (EPICXV).

FIG. 25A shows IFNg secretion as determined by ELISA 3 days after treatment of wildtype HEK293T cells with dCasMini-effector fusions and sgRNA targeting human IFNG.

FIG. 25B shows CD45 surface expression as determined by flow cytometry 2 days after treatment of wildtype HEK293T cells with dCasMini-effector fusions and sgRNA targeting CD45.

FIG. 25C shows CD2 surface expression as determined by flow cytometry 3 days after treatment of wildtype HEK293T cells with dCasMini-effector fusions and sgRNA targeting CD2.

FIG. 25D shows CD2 surface expression as determined by flow cytometry 5 days after treatment of wildtype HEK293T cells with dCasMini-effector fusions and sgRNA targeting CD2.

FIG. 26A shows GFP reporter expression of HEK293T cells bearing a stably integrated TRE3G promoter-driven GFP 2 days after treatment with dCasMini-effector fusions and sgRNA targeting the reporter, as determined by flow cytometry.

FIG. 26B shows GFP reporter expression of HEK293T cells bearing a stably integrated GFP synthetic reporter driven by low-expression miniCMV promoter 2 days after treatment with dCasMini-effector fusions and sgRNA targeting the reporter, as determined by flow cytometry.

FIG. 27A shows GFP reporter expression of HEK293T cells bearing a stably integrated GFP synthetic reporter driven by high-expression EF1u promoter 5 days after treatment with dCasMini-effector fusions and sgRNA targeting the reporter, as determined by flow cytometry.

FIG. 27B shows GFP reporter expression of HEK293T cells bearing a stably integrated GFP synthetic reporter driven by high-expression EF1u promoter 5 days after treatment with dCas9-effector fusions and sgRNA targeting the reporter, as determined by flow cytometry.

FIG. 28A summarizes the effect of heterologous gene effectors on expression of the endogenous gene CXCR4 3, 7, 15, and 28 days after transfection with dCasMini-effector fusions and sgRNA targeting CXCR4, as determined by flow cytometry.

FIG. 28B shows normalized CXCR4 expression values 3, 7, 15, and 28 days after transfection of HEK293T cells with plasmids encoding complexes that comprise heterologous gene effectors disclosed herein or controls targeted to CXCR4 by a sgRNA. Effectors are ranked by effect size at each time point.

FIG. 29A shows the effect of candidate heterologous gene effectors disclosed herein compared to control effectors (dashed lines) on gene expression over time. The effectors shown are fused to dCasMini.

FIG. 29B provides representative flow cytometry histograms for positive control dCas9-KAL, a construct for persistent repression comprising KRAB, DNMT3A, and DNMT3L domains fused to dCas9, showing increased repression of target gene expression over progressive time points.

FIG. 29C provides representative flow cytometry histograms comparing effectors fused to dCasMini. Negative control (dCasMini without modulator) and positive control repressors are shown as compared to a novel suppressor (EPICXV.67).

FIG. 29D shows measurements demonstrating expression of dCasMini-effector over time (as reflected by an mCherry reporter) following plasmid transfection in ESR-GFP HEK293T cells.

FIG. 29E provides bar charts comparing relative sizes (bp) of a positive control fusion (KAL) and its constituent components, as compared to the novel compact modulators presently screened (prefixed EPICXV).

FIG. 29F provides bar charts comparing relative sizes (bp) of a dCas9-KAL control for persistent suppression of gene expression, relative to the present dCasMini-EPICXV constructs tested herein.

FIG. 29G shows normalized reporter GFP fluorescence values for each experimental replicate of positive controls and selected candidate heterologous gene effectors (EPICXVs) at 77 d.p.t. for ESR-GFP cells.

FIG. 30 shows a correlation of normalized GFP fluorescence in ESR-GFP synthetic reporter cells (averaged across all time points from 3 to 77 d.p.t.; y-axis) and normalized CXCR4-APC fluorescence (averaged across all time points from 3 to 28 d.p.t.; x-axis).

### DETAILED DESCRIPTION

Gene expression underpins various physiological and pathological effects in cells and tissues, contributing to many diseases and conditions, thus agents that modulate expression of specific genes in a desirable way could have therapeutic benefit.

Developing agents that elicit, robust, persistent, and/or reversible changes in gene expression has proven challenging, however, as many candidate therapeutics achieve only modest or short lived effects, or conversely result in off-target effects. Additionally, many current approaches to gene editing and genome engineering can result in off-target effects that can be associated with undesirable toxicity profiles, and in some cases, undesirable effects can be permanent. There is thus a need for novel strategies to regulate gene expression that allow robust, persistent, and/or reversible modulation of target gene expression and activity, for example, expression of genes that impact human disease.

Molecular tools that act in an epigenetic fashion have the potential to elicit efficient, durable, and low-risk therapeutic modulation of gene expression. In some embodiments, the disclosure provides compositions and methods for identifying novel effector domains capable of efficient, effective, and persistent epigenetic modification of target gene expression. For example, provided are heterologous gene effector domains and complexes of the effector domains with guide moieties to modulate (e.g., upregulate or downregulate) expression or activity of particular target gene(s) (e.g., target endogenous gene(s)).

Combinations of heterologous gene effectors also have the potential to achieve advantageous outcomes in regulating gene expression, for example, via synergistic effects. In some embodiments, the disclosure provides compositions and methods comprising novel combinations of heterologous gene effectors, for example, combinations of effector domains that are chromatic regulators and/or transcriptional regulators from the human nuclear proteome, viral sources, and/or other types of heterologous gene effectors disclosed herein.

Context-dependent effects present an additional challenge for therapeutic modulation of gene expression and activity. For example, a strategy or transcriptional modulator that achieves a desirable effect on expression of a particular target gene (e.g., a particular target endogenous gene), cell type, subject, etc. may not achieve the desired effect for a different target gene (e.g., a different target endogenous gene), cell type, or subject. In some cases, systems and methods of the disclosure are customized to or are applicable to or are particularly suited to a specific target gene (e.g., a specific target endogenous gene), a specific cell type, a specific target disease, a specific subject, etc.

In some embodiments, the disclosure provides compositions and methods for high throughput screens to identify heterologous gene effector domains and complexes of the effector domains with guide moieties to modulate expression of particular target gene(s) (e.g., particular target endogenous gene(s)). In some embodiments, the disclosure provides complexes identified by systems disclosed herein that can be employed for other purposes, such as research or as therapeutics.

### Heterologous Gene Effectors

The disclosure provides compositions, systems, methods and methods (for example, complexes and libraries) that utilize heterologous gene effectors (e.g., gene effectors that are heterologous to a cell comprising the gene effectors and/or another component in a complex of the disclosure). Heterologous gene effectors comprise domains that are capable of, or are candidates for, modulating expression of a target gene (e.g., a target endogenous gene), for example, activating, repressing, upregulating, downregulating, or stabilizing an expression level or activity level of the gene. Heterologous gene effectors can be heterologous with respect to another component that is present in a complex, for example, a guide moiety (e.g., nuclease and/or guide nucleic acid, as disclosed herein). In some cases, heterologous gene effectors can be heterologous with respect to a host cell they are introduced to.

A heterologous gene effector can be or can comprise a sequence from any suitable source, for example, an amino acid sequence from a human protein, viral protein, or other protein as disclosed herein. A heterologous gene effector can be or can comprise a sequence from a protein that primarily localized to the nucleus, for example, a member of the human nuclear proteome. A heterologous gene effector can be or can comprise one or more natural amino acid residues. A heterologous gene effector can be or can comprise one or more synthetic amino acid residues.

A heterologous gene effector can be or can comprise a sequence from a mammalian protein. A heterologous gene effector can be or can comprise a sequence from a human protein. A heterologous gene effector can be or can comprise a sequence from a viral protein. A heterologous gene effector can be or can comprise a sequence from a non-human primate protein. A heterologous gene effector can be or can comprise a sequence from a non-human mammal protein. A heterologous gene effector can be or can comprise a sequence from a non-rodent mammal protein. A heterologous gene effector can be or can comprise a sequence from a plant protein. A heterologous gene effector can be or can comprise a sequence from a pig protein. A heterologous gene effector can be or can comprise a sequence from a lagomorph protein. A heterologous gene effector can be or can comprise a sequence from a canine protein. A heterologous gene effector can be or can comprise a sequence from an avian protein. A heterologous gene effector can be or can comprise a sequence from a reptilian protein. A heterologous gene effector can be or can comprise a sequence from a bacterial protein. A heterologous gene effector can be or can comprise a sequence from an archaeal protein.

A heterologous gene effector can be or can comprise a sequence from a chromatin regulator (CR). Chromatin regulators include functional domains from various classes of histone and DNA modifying enzymes (e.g., DNMTs, HATs, HMTs, etc.).

A heterologous gene effector can comprise two or more domains from chromatin regulators, e.g., located at a C-terminus, an N-terminus, or within a polypeptide sequence, in tandem or separate.

In some embodiments, a heterologous gene effector is one that facilitates heterochromatin formation. Non-limiting examples of proteins that can facilitate heterochromatin formation include HP1α, HP1β, KAP1, KRAB, SUV39H1, and G9a.

In some embodiments, a heterologous gene effector modulates histones through methylation. In some embodiments, a heterologous gene effector modulates histones through acetylation. In some embodiments, a heterologous gene effector modulates histones through phosphorylation. In some embodiments, a heterologous gene effector modulates histones through ADP-ribosylation. In some embodiments, a heterologous gene effector modulates histones through glycosylation. In some embodiments, a heterologous gene effector modulates histones through SUMOylation. In some embodiments, a heterologous gene effector modulates histones through ubiquitination. In some embodiments, a heterologous gene effector modulates histones by remodeling histone structure, e.g., via an ATP hydrolysis-dependent process.

In some embodiments, a heterologous gene effector facilitates spatial positioning of proteins on or near the target polynucleotide, e.g., transcriptional repressors, transcription factors, histones, etc. In some embodiments, a heterologous gene effector is useful for manipulating the spatiotemporal organization of genomic DNA and RNA components in the nucleus and/or cytoplasm, e.g., for regulating diverse cellular functions.

In some embodiments, a heterologous gene effector is from a family of related histone acetyltransferases. Non-limiting examples of histone acetyltransferases include GNAT subfamily, MYST subfamily, p300/CBP subfamily, HAT1 subfamily, GCN5, PCAF, Tip60, MOZ, MORF, MOF, HBO1, p300, CBP, HAT1, ATF-2, SRC1, and TAFII250.

In some embodiments, a heterologous gene effector is from a histone lysine methyltransferase. Non-limiting examples of histone lysine methyltransferases include EZH subfamily, Non-SET subfamily, Other SET subfamily, PRDM subfamily, SET1 subfamily, SET2 subfamily, SUV39 subfamily, SYMD subfamily, ASH1L, EHMT1, EHMT2, EZH1, EZH2, MLL, MLL2, MLL3, MLL4, MLL5, NSD1, NSD2, NSD3, PRDM1, PRDM10, PRDM11, PRDM12, PRDM13, PRDM14, PRDM15, PRDM16, PRDM2, PRDM4, PRDM5, PRDM6, PRDM7, PRDM8, PRDM9, SET1, SET1L, SET2L, SETD2, SETD3, SETD4, SETD5, SETD6, SETD7, SETD8, SETDB1, SETDB2, SETMAR, SUV39H1, SUV39H2, SUV420H1, SUV420H2, SYMD1, SYMD2, SYMD3, SYMD4, and SYMD5.

In some embodiments, a heterologous gene effector is from a component of a chromatin remodeling complex. In some embodiments, a heterologous gene effector is a component of BAF, for example, Actin, ARIDA/B, BAF155, BAF170, BAF45 A/B/C/D, BAF53 A/B, BAF57, BAF60 A/B/C, BRG1/BRM, INIl, or SS18.

In some embodiments, a heterologous gene effector is from a component of PBAF, for example, Actin, ARID2, BAF155, BAF170, BAF180, BAF45 A/B/C/D, BAF53 A/B, BAF57, BAF60 A/B/C, BRD7, BRG1, or INIl.

In some embodiments, a heterologous gene effector is from a component of an ISWI family chromatin remodeling complex, for example, ACF subfamily, RSF subfamily, CERF subfamily, CHRAC subfamily, NURF subfamily, NoRC subfamily, WICH subfamily, b-WICH subfamily, ACF1, ATPase, BPTF, CECR2, CHRAC15, CHRAC17, CSB, DEK, MYBBPlA, NM1, RBAP46/48, RHII/Gua, RSF1, SAP155, SNF2H, SNF2H/L, SNF2L, TIP5, or WSTF.

In some embodiments, a heterologous gene effector is from a component of a CHD family complex, for example, a NuRD complex, NuRD-like complex, or CHD complex. In some embodiments, a heterologous gene effector is from CHD1/2/6/7/8/9, CHD3/4, CHD5, GATAD2 A/B, GATAD2 B, HDAC1, HDAC2, HDAC2, MBD2/3, MTA1/2/3, MTA3, or RBAP46, RBAP46/48.

In some embodiments, a heterologous gene effector is from a component of an IN080 family complex, for example, from an IN080 complex, Tip60/p400 complex, SRCAP complex, AMIDA, ARP6, BAF53, BAF53, BAF53A, BRD8, DMAP1, DMAP1, EPC1/2, FLJ11730, GAS41, GAS41, IES2, IES6, ING3, IN080, IN080E, MCRS1, MRG15, MRGBP, MRGX, NFRKB, p400, RUVBL1/2, RUVBL1/2, RUVBL1/2, SRCAP, Tip60, TRRAP, UCH37, YL-1, YL-1, YY1, or ZnF-HIT1.

A heterologous gene effector can be or can comprise a sequence from a transcriptional regulator (TR). TR gene effectors include transcriptional regulatory domains from various families of transcription factors (e.g. KRAB, p65, MED, GTFs, etc.).

A heterologous gene effector can comprise a transcriptional activator domain. A heterologous gene effector can comprise can comprise two or more tandem transcriptional activation domains, e.g., located at a C-terminus, an N-terminus, or within a polypeptide sequence.

Non-limiting examples of transcriptional activation domains include GAL4, herpes simplex activation domain VP16, VP64 (a tetramer of the herpes simplex activation domain VP16), NF-KB p65 subunit, Epstein-Barr virus R transactivator (Rta). Examples of transcriptional activation domains are described in Chavez et al., Nat Methods, 2015, 12(4):326-328 and U.S. Patent App. Publ. No. 20140068797. In some embodiments, such transcriptional activation domains are used as controls in methods of the disclosure. In some embodiments, such transcriptional activation domains are used as one heterologous gene effector in a complex that comprises at least one additional heterologous gene effector (e.g., a different effector).

A heterologous gene effector can comprise a transcriptional repressor domain. A heterologous gene effector can comprise two or more transcriptional repressor domains, e.g., located at a C-terminus, an N-terminus, or within a polypeptide sequence, in tandem or separate.

Non-limiting examples of transcriptional repressor domains include the KRAB (Kruppel-associated box) domain of Koxl, the Mad mSIN3 interaction domain (SID), and ERF repressor domain (ERD). Examples of transcriptional repressor domains are described in in Chavez et al., Nat Methods, 2015, 12(4):326-328 and U.S. Patent App. Publ. No. 20140068797. In some embodiments, such transcriptional repressor domains are used as controls in methods of the disclosure. In some embodiments, such transcriptional repressor domains are used as one heterologous gene effector in a complex that comprises at least one additional heterologous gene effector (e.g., a different effector).

In some embodiments, a heterologous gene effector is from a gene product that is a transcription factor.

In some embodiments, a heterologous gene effector is from a gene product that is a hematopoietic stem cell transcription factor. Non-limiting examples of hematopoietic stem cell transcription factors include AHR, Aiolos/IKZF3, CDX4, CREB, DNMT3A, DNMT3B, EGR1, Fox03, GATA-1, GATA-2, GATA-3, Helios, HES-1, HHEX, HIF-1 alpha/HIF1A, HMGB1/HMG-1, HMGB3, Ikaros, c-Jun, LMO2, LMO4, c-Maf, MafB, MEF2C, MYB, c-Myc, NFATC2, NFIL3/E4BP4, Nrf2, p53, PITX2, PRDM16/MEL1, Prox1, PU.1/Spi-1, RUNX1/CBFA2, SALL4, SCL/Tall, Smad2, Smad2/3, Smad4, Smad7, Spi-B, STAT Activators, STAT Inhibitors, STAT3, STAT4, STAT5a, STAT6, and TSC22.

In some embodiments, a heterologous gene effector is from a gene product that is a mesenchymal stem cell transcription factor. Non-limiting examples of mesenchymal stem cell transcription factors include DUX4, DUX4/DUX4c, DUX4c, EBF-1, EBF-2, EBF-3, ETV5, FoxC2, FoxF1, GATA-4, GATA-6, HMGA2, c-Jun, MYF-5, Myocardin, MyoD, Myogenin, NFATC2, p53, Pax3, PDX-1/IPF1, PLZF, PRDM16/MEL1, RUNX2/CBFA1, Smad1, Smad3, Smad4, Smad5, Smad8, Smad9, Snail, SOX2, SOX9, SOX11, STAT Activators, STAT Inhibitors, STAT1, STAT3, TBX18, Twist-1, and Twist-2.

In some embodiments, a heterologous gene effector is from a gene product that is an embryonic stem cell transcription factor. Non-limiting examples of embryonic stem cell transcription factors include Brachyury, EOMES, FoxC2, FoxD3, FoxF1, FoxH1, Fox01/FKHR, GATA-2, GATA-3, GBX2, Goosecoid, HES-1, HNF-3 alpha/FoxAl, c-Jun, KLF2, KLF4, KLF5, c-Maf, Max, MEF2C, MIXL1, MTF2, c-Myc, Nanog, NFkB/IkB Activators, NFkB/IkB Inhibitors, NFkB1, NFkB2, Oct-3/4, Otx2, p53, Pax2, Pax6, PRDM14, Rex-1/ZFP42, SALL1, SALL4, Smad1, Smad2, Smad2/3, Smad3, Smad4, Smad5, Smad8, Snail, SOX2, SOX7, SOX15, SOX17, STAT Activators, STAT Inhibitors, STAT3, SUZ12, TBX6, TCF-3/E2A, THAP11, UTF1, WDR5, WT1, ZNF206, and ZNF281.

In some embodiments, a heterologous gene effector is from a gene product that is an induced pluripotent stem cell (iPSC) transcription factor. Non-limiting examples of iPSC transcription factors include KLF2, KLF4, c-Maf, c-Myc, Nanog, Oct-3/4, p53, SOX1, SOX2, SOX3, SOX15, SOX18, and TBX18.

In some embodiments, a heterologous gene effector is from a gene product that is an epithelial stem cell transcription factor. Non-limiting examples of epithelial stem cell transcription factors include ASCL2/Mash2, CDX2, DNMT1, ELF3, Ets-1, FoxM1, FoxNI, GATA-6, Hairless, HNF-4 alpha/NR2A1, IRF6, c-Maf, MITF, Miz-1/ZBTB17, MSX1, MSX2, MYB, c-Myc, Neurogenin-3, NFATC1, NKX3.1, Nrf2, p53, p63/TP73L, Pax2, Pax3, RUNX1/CBFA2, RUNX2/CBFA1, RUNX3/CBFA3, Smadi, Smad2, Smad2/3, Smad4, Smad5, Smad7, Smad8, Snail, SOX2, SOX9, STAT Activators, STAT Inhibitors, STAT3, SUZ12, TCF-3/E2A, and TCF7/TCF1.

In some embodiments, a heterologous gene effector is from a gene product that is a cancer stem cell transcription factor. Non-limiting examples of cancer stem cell transcription factors include Androgen R/NR3C4, AP-2 gamma, beta-Catenin, beta-Catenin Inhibitors, Brachyury, CREB, ER alpha/NR3A1, ER beta/NR3A2, FoxM1, Fox03, FRA-1, GLI-1, GLI-2, GLI-3, HIF-1 alpha/HIF1A, HIF-2 alpha/EPAS1, HMGAIB, c-Jun, JunB, KLF4, c-Maf, MCM2, MCM7, MITF, c-Myc, Nanog, NFkB/IkB Activators, NFkB/IkB Inhibitors, NFkBI, NKX3.1, Oct-3/4, p53, PRDM14, Snail, SOX2, SOX9, STAT Activators, STAT Inhibitors, STAT3, TAZ/WWTR1, TBX3, Twist-1, Twist-2, WTi, and ZEBi.

In some embodiments, a heterologous gene effector is from a gene product that is a cancer-related transcription factor. Non-limiting examples of cancer-related transcription factors include ASCL1/Mashi, ASCL2/Mash2, ATF1, ATF2, ATF4, BLIMP1/PRDM1, CDX2, CDX4, DLX5, DNMT1, E2F-1, EGR1, ELF3, Ets-1, FosB/GOS3, FoxCi, FoxC2, FoxFI, GADD153, GATA-2, HMGA2, HMGB1/HMG-1, HNF-3 alpha/FoxAl, HNF-6/ONECUT1, HSF1, ID1, ID2, JunD, KLF10, KLF12, KLF17, LMO2, MEF2C, MYCL1/L-Myc, NFkB2, Oct-1, p63/TP73L, Pax3, PITX2, Proxi, RAP80, Rex-1/ZFP42, RUNX1/CBFA2, RUNX3/CBFA3, SALL4, SCL/Tall, Sirtuin 2/SIRT2, Smad3, Smad4, Smad5, SOX11, STAT5a/b, STAT5a, STAT5b, TCF7/TCF1, TORC1, TORC2, TRIM32, TRPS1, and TSC22.

In some embodiments, a heterologous gene effector is from a gene product that is an immune cell transcription factor. Non-limiting examples of immune cell transcription factors include AP-1, Bcl6, E2A, EBF, Eomes, FoxP3, GATA3, Id2, Ikaros, IRF, IRF1, IRF2, IRF3, IRF3, IRF7, NFAT, NFkB, Pax5, PLZF, PU.1, ROR-gamma-T, STAT, STAT1, STAT2, STAT3, STAT4, STAT5, STAT5A, STAT5B, STAT6, T-bet, TCF7, and ThPOK.

In some embodiments, a heterologous gene effector is from a gene product that is a RNA polymerase related protein. In some embodiments, a heterologous gene effector is from a transcription factor with a basic domain. In some embodiments, a heterologous gene effector is from a transcription factor with a zinc-coordinated DNA binding domain. In some embodiments, a heterologous gene effector is from a transcription factor with a helix-turn-helix domain. In some embodiments, a heterologous gene effector is from a transcription factor with an alpha helical DNA binding domain. In some embodiments, a heterologous gene effector is from a transcription factor with an alpha helix exposed by beta structures. In some embodiments, a heterologous gene effector is from a transcription factor with an immunoglobulin fold. In some embodiments, a heterologous gene effector is from a transcription factor with a with a beta-Hairpin exposed by an alpha/beta-scaffold. In some embodiments, a heterologous gene effector is from a transcription factor with a beta sheet binding to DNA. In some embodiments, a heterologous gene effector is from a transcription factor with a beta barrel DNA binding domain.

In some embodiments, a heterologous gene effector is from a gene product that is a nuclear receptor, for example, a nuclear hormone receptor. Non-limiting examples of nuclear hormone receptors include those encoded by NROB1, NROB2, NR1A1, NR1A2, NR1B1, NR1B2, NR1B3, NR1C1, NR1C2, NR1C3, NR1D1, NR1D2, NR1F1, NR1F2, NR1F3, NR1H4, NR1H5, NR1H3, NR1H2, NR1Il, NR1I2, NR1I3, NR2A1, NR2A2, NR2B1, NR2B2, NR2B3, NR2C1, NR2C2, NR2E1, NR2E3, NR2F1, NR2F2, NR2F6, NR3A1, NR3A2, NR3B1, NR3B2, NR3B3, NR3C4, NR3C1, NR3C2, NR3C3, NR4A1, NR4A2, NR4A3, NR5A1, NR5A2, and NR6A1.

In some embodiments, a heterologous gene effector is from a gene product that is involved in nucleosome assembly. In some embodiments, a heterologous gene effector is from a gene product that is involved in DNA metabolism. In some embodiments, a heterologous gene effector is from a gene product that is involved in nucleotide metabolism. In some embodiments, a heterologous gene effector is from a gene product that is involved in ribosome biogenesis. In some embodiments, a heterologous gene effector is from a gene product that is involved in protein folding. In some embodiments, a heterologous gene effector is from a gene product that is involved in translation. In some embodiments, a heterologous gene effector is from a gene product that is involved in signaling. In some embodiments, a heterologous gene effector is from a gene product that is involved in proteolysis. In some embodiments, a heterologous gene effector is from a gene product that is involved in negative regulation of endopeptidase activity.

In some embodiments, a list of candidate heterologous gene effectors can be stratified by factors such as activation versus repression, target cellular pathway, evolutionary sequence constraint, binding of DNA/RNA/both, protein folding pattern, host/cell tropism, direct/indirect activity, binding promiscuity, nuclear/cytoplasmic action, and other criteria. In some embodiments, such stratifications allow design of a screen library of the disclosure that encompasses a broad spectrum of potential molecular functions, thereby increasing the likelihood for discovery of heterologous gene effectors that are suitable for purposes disclosed herein.

In some embodiments, predictive filtering techniques are applied to candidate heterologous gene effector sequences, e.g., in silico. Predictive filtering techniques can comprise, for example, identification of suitable biophysical properties for core activator domains (e.g., down to 13 bp) through experiments in yeast; the presence of acidic, bulky hydrophobic, alpha helix, and/or negative charge; the presence of motif repeats that may influence duration of effect; and PADDLE-like convolutional neural network/transformer algorithms or similar predictive techniques.

In some embodiments, a heterologous gene effector can regulate expression of a target gene that is exogenous to a host subject, for example, a pathogen target gene or an exogenous gene expressed as a result of a therapeutic intervention.

In some embodiments, a heterologous gene effector comprises, consists essentially of, or consists of a peptide sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.1%, at least about 99.1%, at least about 99.3%, at least about 99.4%, at least about 99.5%, at least about 99.6%, at least about 99.7%, at least about 99.8%, at least about 99.9%, at least about 99.95%, at least about 99.99%, or about 100% sequence identity or sequence similarity to any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052, e.g., over the entire length.

In some embodiments, a heterologous gene effector comprises, consists essentially of, or consists of a peptide sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, at most about 99.1%, at most about 99.1%, at most about 99.3%, at most about 99.4%, at most about 99.5%, at most about 99.6%, at most about 99.7%, at most about 99.8%, at most about 99.9%, at most about 99.95%, at most about 99.99%, or at most about 100% sequence identity or sequence similarity to any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052, e.g., over the entire length.

In some embodiments, a heterologous gene effector comprises, consists essentially of, or consists of a peptide sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, about 99.1%, about 99.1%, about 99.3%, about 99.4%, about 99.5%, about 99.6%, about 99.7%, about 99.8%, about 99.9%, about 99.95%, about 99.99%, or about 100% sequence identity or sequence similarity to any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052, e.g., over the entire length.

In some embodiments, a heterologous gene effector comprises a peptide sequence with one or more amino acid insertions, deletions, or substitutions compared to any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052.

For example, a heterologous gene effector can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052.

In some embodiments, a heterologous gene effector comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any amino acid sequence disclosed herein, for example, any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052.

In some embodiments, a heterologous gene effector comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052.

The one or more insertions can be at the N-terminus, C-terminus, within the amino acid sequence, or a combination thereof. The one or more insertions can be contiguous, non-contiguous, or a combination thereof.

In some embodiments, a heterologous gene effector comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid deletions relative to any amino acid sequence disclosed herein, for example, any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052.

In some embodiments, a heterologous gene effector comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any amino acid sequence disclosed herein, for example, any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052.

In some embodiments, a heterologous gene effector comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any amino acid sequence disclosed herein, for example, any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052.

The one or more deletions can be at the N-terminus, C-terminus, within the amino acid sequence, or a combination thereof. The one or more deletions can be contiguous, non-contiguous, or a combination thereof.

In some embodiments, a heterologous gene effector comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to any amino acid sequence disclosed herein, for example, any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052.

In some embodiments, a heterologous gene effector comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any amino acid sequence disclosed herein, for example, any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052.

In some embodiments, a heterologous gene effector comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any amino acid sequence disclosed herein, for example, any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052.

The one or more substitutions can be at the N-terminus, C-terminus, within the amino acid sequence, or a combination thereof. The one or more substitutions can be contiguous, non-contiguous, or a combination thereof. The one or more substitutions can comprise a substitution of an N-terminal methionine for a different residue, for example, Leucine.

In some embodiments a heterologous gene effector does not contain an N-terminal methionine. In some embodiments, an N-terminal methionine found in any one of SEQ ID NOs: 16-16154, 16-13605, 16155-47350, 16155-43953, 47351-49333, or 16-49333 is deleted, absent, or substituted for a different residue. For example, SEQ ID NOs: 49353-50052 provide illustrative examples of sequences in which an N-terminal methionine has been substituted for an N-terminal leucine. In some embodiments a heterologous gene effector comprises an N-terminal methionine.

The one or more substitutions can be conservative, non-conservative, or a combination thereof. A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to any one of SEQ ID NOs: 1102, 2057, 5543, 9066, 11948, 15646, 17629, 19860, 21015, 21166, 22149, 22707, 23631, 23639, 25430, 25555, 32678, 33890, 34047, 35737, 38138, 38780, 40913, 40985, 40986, and 42623.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 1102.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 2057.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 5543.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 9066.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 11948.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 15646.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 17629.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 19860.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 21015.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 21166.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 22149.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 22707.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 23631.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 23639.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 25430.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 25555.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 32678.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 33890.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 34047.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 35737.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 38138.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 38780.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 40913.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 40985.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 40986.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to SEQ ID NO: 42623.

In some embodiments, a heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to any one of SEQ ID NOs: 16, 17, 23, 26, 35, 66, 67, 71, 74, 126, 148, 159, 160, 166, 171, 172, 174, 176, 178, 185, 188, 189, 210, 225, 237, 240, 245, 253, 259, 298, 302, 308, 316, 334, 335, 360, 362, 375, 377, 394, 398, 415, 426, 460, 476, 480, 486, 515, 527, 539, 548, 565, 571, 604, 630, 643, 676, 720, 734, 746, 774, 797, 812, 833, 835, 841, 852, 871, 874, 879, 881, 927, 934, 947, 952, 967, 986, 993, 1009, 1033, 1042, 1044, 1054, 1075, 1076, 1077, 1079, 1084, 1094, 1102, 1111, 1113, 1120, 1129, 1131, 1149, 1156, 1161, 1186, 1190, 1225, 1253, 1258, 1261, 1262, 1265, 1289, 1295, 1299, 1303, 1308, 1311, 1313, 1315, 1328, 1350, 1355, 1368, 1374, 1375, 1380, 1388, 1395, 1400, 1406, 1424, 1427, 1430, 1437, 1440, 1493, 1498, 1502, 1510, 1511, 1519, 1522, 1581, 1591, 1597, 1615, 1630, 1644, 1662, 1675, 1714, 1718, 1720, 1730, 1735, 1745, 1778, 1785, 1791, 1803, 1812, 1820, 1828, 1835, 1869, 1870, 1888, 1889, 1908, 1911, 1912, 1940, 1980, 1981, 2011, 2046, 2057, 2086, 2109, 2119, 2192, 2204, 2233, 2247, 2257, 2276, 2285, 2296, 2304, 2309, 2316, 2335, 2338, 2339, 2347, 2352, 2378, 2382, 2394, 2397, 2429, 2493, 2507, 2519, 2534, 2557, 2572, 2595, 2642, 2650, 2681, 2707, 2725, 2767, 2768, 2804, 2807, 2825, 2837, 2865, 2878, 2888, 2889, 2899, 2966, 2980, 3069, 3073, 3078, 3081, 3093, 3116, 3130, 3175, 3191, 3217, 3222, 3225, 3244, 3254, 3255, 3256, 3261, 3287, 3292, 3299, 3309, 3330, 3353, 3361, 3363, 3379, 3383, 3389, 3392, 3402, 3407, 3416, 3424, 3425, 3444, 3445, 3457, 3462, 3486, 3508, 3528, 3531, 3535, 3541, 3550, 3561, 3563, 3572, 3586, 3590, 3597, 3608, 3630, 3714, 3718, 3723, 3726, 3730, 3735, 3764, 3771, 3786, 3803, 3804, 3851, 3869, 3873, 3907, 3911, 3914, 3918, 3926, 3946, 3965, 4010, 4016, 4025, 4032, 4047, 4063, 4065, 4072, 4082, 4131, 4143, 4150, 4161, 4201, 4225, 4231, 4239, 4261, 4274, 4284, 4289, 4295, 4298, 4325, 4327, 4346, 4358, 4362, 4368, 4444, 4459, 4516, 4547, 4549, 4562, 4571, 4598, 4610, 4618, 4640, 4653, 4669, 4684, 4706, 4721, 4751, 4753, 4763, 4770, 4776, 4807, 4812, 4820, 4826, 4852, 4857, 4867, 4880, 4886, 4892, 4899, 4958, 4961, 4972, 4991, 4998, 5002, 5005, 5006, 5009, 5025, 5027, 5028, 5038, 5045, 5055, 5062, 5081, 5093, 5095, 5129, 5169, 5172, 5189, 5211, 5219, 5245, 5250, 5272, 5273, 5274, 5285, 5294, 5306, 5307, 5351, 5359, 5395, 5402, 5405, 5412, 5444, 5448, 5497, 5502, 5524, 5543, 5559, 5570, 5577, 5604, 5612, 5646, 5649, 5655, 5667, 5697, 5699, 5724, 5734, 5735, 5743, 5747, 5758, 5764, 5782, 5783, 5849, 5856, 5858, 5875, 5898, 5901, 5913, 5921, 5928, 5954, 5959, 5963, 5969, 5980, 5985, 5998, 6003, 6021, 6033, 6036, 6050, 6052, 6073, 6075, 6102, 6107, 6121, 6134, 6158, 6173, 6179, 6182, 6198, 6205, 6236, 6272, 6276, 6298, 6310, 6332, 6335, 6356, 6384, 6386, 6389, 6414, 6431, 6446, 6447, 6470, 6502, 6508, 6522, 6533, 6545, 6558, 6580, 6588, 6604, 6608, 6610, 6628, 6632, 6648, 6651, 6652, 6659, 6673, 6681, 6682, 6698, 6699, 6752, 6757, 6761, 6795, 6813, 6818, 6819, 6828, 6838, 6842, 6848, 6868, 6873, 6874, 6882, 6893, 6895, 6900, 6902, 6903, 6928, 6931, 6941, 6952, 6953, 6955, 6984, 7027, 7029, 7030, 7034, 7070, 7076, 7106, 7115, 7128, 7140, 7143, 7166, 7171, 7187, 7226, 7259, 7279, 7281, 7286, 7291, 7296, 7304, 7320, 7325, 7336, 7375, 7391, 7405, 7414, 7432, 7433, 7443, 7462, 7489, 7500, 7501, 7508, 7520, 7551, 7570, 7576, 7590, 7596, 7608, 7611, 7613, 7621, 7625, 7637, 7643, 7651, 7661, 7696, 7701, 7703, 7705, 7711, 7717, 7722, 7728, 7736, 7770, 7781, 7803, 7807, 7809, 7831, 7833, 7834, 7856, 7888, 7890, 7894, 7926, 7927, 7943, 7949, 7965, 7984, 8010, 8018, 8027, 8028, 8041, 8068, 8089, 8097, 8102, 8105, 8111, 8129, 8149, 8186, 8189, 8219, 8223, 8227, 8236, 8243, 8249, 8255, 8256, 8258, 8259, 8272, 8278, 8280, 8283, 8297, 8301, 8308, 8314, 8324, 8328, 8347, 8357, 8363, 8371, 8386, 8394, 8408, 8420, 8421, 8429, 8432, 8436, 8437, 8439, 8468, 8485, 8488, 8489, 8505, 8538, 8551, 8563, 8564, 8574, 8579, 8628, 8629, 8631, 8634, 8636, 8637, 8641, 8670, 8692, 8699, 8729, 8730, 8752, 8779, 8805, 8841, 8854, 8865, 8886, 8893, 8931, 8933, 8973, 8981, 8985, 8998, 9000, 9003, 9017, 9021, 9024, 9026, 9027, 9040, 9050, 9053, 9055, 9066, 9069, 9072, 9078, 9094, 9095, 9100, 9110, 9119, 9130, 9152, 9153, 9189, 9194, 9222, 9223, 9225, 9231, 9262, 9264, 9267, 9273, 9283, 9297, 9304, 9307, 9314, 9320, 9324, 9328, 9338, 9380, 9386, 9397, 9403, 9413, 9442, 9472, 9516, 9528, 9534, 9542, 9552, 9568, 9602, 9611, 9626, 9632, 9648, 9649, 9663, 9667, 9669, 9670, 9672, 9673, 9675, 9678, 9682, 9700, 9741, 9750, 9761, 9771, 9829, 9845, 9853, 9854, 9879, 9880, 9891, 9893, 9894, 9899, 9901, 9903, 9922, 9933, 9937, 9940, 9943, 9948, 9962, 9965, 9971, 9988, 9990, 10007, 10014, 10016, 10025, 10040, 10058, 10069, 10072, 10095, 10097, 10113, 10123, 10160, 10230, 10234, 10235, 10236, 10238, 10250, 10252, 10279, 10289, 10292, 10309, 10313, 10314, 10315, 10316, 10329, 10348, 10357, 10364, 10383, 10394, 10405, 10407, 10414, 10420, 10421, 10423, 10427, 10428, 10436, 10437, 10439, 10446, 10451, 10459, 10463, 10466, 10489, 10490, 10491, 10512, 10516, 10555, 10566, 10570, 10584, 10587, 10588, 10607, 10615, 10624, 10637, 10648, 10663, 10701, 10732, 10759, 10778, 10788, 10796, 10813, 10853, 10865, 10878, 10919, 10943, 10945, 10950, 10955, 10957, 10969, 10984, 10991, 10998, 10999, 11018, 11019, 11027, 11035, 11036, 11042, 11045, 11079, 11090, 11095, 11106, 11129, 11187, 11207, 11218, 11223, 11233, 11260, 11265, 11272, 11290, 11297, 11306, 11326, 11374, 11386, 11396, 11411, 11432, 11437, 11455, 11459, 11471, 11481, 11485, 11486, 11506, 11511, 11550, 11555, 11557, 11577, 11601, 11621, 11642, 11652, 11670, 11673, 11705, 11709, 11712, 11713, 11715, 11716, 11723, 11730, 11737, 11741, 11749, 11750, 11778, 11784, 11785, 11794, 11797, 11799, 11804, 11810, 11835, 11888, 11893, 11895, 11904, 11931, 11932, 11948, 11969, 11982, 11985, 12020, 12024, 12029, 12055, 12061, 12066, 12067, 12069, 12072, 12096, 12114, 12121, 12132, 12161, 12183, 12187, 12191, 12199, 12227, 12254, 12266, 12344, 12345, 12362, 12393, 12400, 12401, 12422, 12427, 12436, 12448, 12451, 12476, 12498, 12526, 12542, 12558, 12560, 12567, 12570, 12590, 12591, 12609, 12613, 12626, 12632, 12660, 12669, 12671, 12681, 12692, 12706, 12716, 12722, 12723, 12738, 12753, 12757, 12771, 12775, 12783, 12786, 12806, 12809, 12822, 12838, 12843, 12846, 12847, 12892, 12904, 12925, 12928, 12932, 12945, 12947, 12958, 12960, 12965, 12982, 13022, 13027, 13031, 13032, 13038, 13046, 13083, 13092, 13124, 13149, 13174, 13190, 13195, 13198, 13211, 13219, 13226, 13229, 13233, 13273, 13283, 13288, 13327, 13345, 13363, 13364, 13385, 13394, 13395, 13403, 13420, 13452, 13497, 13509, 13517, 13528, 13553, 13569, 16179, 16218, 16224, 16251, 16278, 16313, 16319, 16335, 16364, 16381, 16414, 16440, 16472, 16482, 16501, 16505, 16506, 16514, 16515, 16516, 16524, 16535, 16541, 16578, 16618, 16667, 16677, 16699, 16702, 16705, 16769, 16771, 16786, 16807, 16856, 16867, 16916, 16935, 17012, 17029, 17032, 17054, 17067, 17084, 17087, 17107, 17115, 17121, 17127, 17142, 17146, 17193, 17196, 17209, 17231, 17247, 17256, 17264, 17268, 17273, 17275, 17298, 17314, 17326, 17332, 17349, 17354, 17377, 17385, 17434, 17444, 17447, 17469, 17475, 17491, 17494, 17511, 17528, 17538, 17546, 17556, 17563, 17565, 17570, 17608, 17613, 17629, 17633, 17639, 17666, 17674, 17691, 17697, 17735, 17793, 17816, 17817, 17821, 17842, 17843, 17883, 17901, 17928, 17950, 17956, 17961, 17965, 17983, 18026, 18092, 18106, 18113, 18125, 18144, 18162, 18221, 18237, 18340, 18351, 18355, 18388, 18439, 18444, 18468, 18499, 18504, 18555, 18561, 18579, 18587, 18588, 18599, 18618, 18642, 18664, 18696, 18725, 18736, 18741, 18787, 18809, 18827, 18854, 18864, 18872, 18888, 18936, 19013, 19034, 19048, 19063, 19075, 19078, 19079, 19092, 19123, 19126, 19156, 19188, 19211, 19212, 19235, 19237, 19239, 19248, 19261, 19289, 19292, 19320, 19325, 19387, 19389, 19424, 19435, 19445, 19490, 19512, 19542, 19556, 19559, 19568, 19571, 19581, 19583, 19591, 19599, 19621, 19624, 19633, 19642, 19671, 19684, 19711, 19731, 19738, 19745, 19761, 19771, 19781, 19809, 19815, 19858, 19860, 19877, 19888, 19916, 19973, 19989, 19998, 20006, 20028, 20055, 20100, 20101, 20116, 20144, 20169, 20207, 20266, 20273, 20285, 20316, 20317, 20321, 20342, 20352, 20365, 20375, 20383, 20387, 20390, 20415, 20416, 20429, 20484, 20506, 20521, 20526, 20541, 20556, 20578, 20637, 20657, 20668, 20703, 20746, 20767, 20818, 20839, 20857, 20871, 20876, 20877, 20878, 20881, 20887, 20939, 20946, 20993, 20998, 21001, 21004, 21015, 21024, 21042, 21054, 21057, 21091, 21123, 21126, 21134, 21139, 21149, 21166, 21211, 21229, 21238, 21291, 21294, 21295, 21306, 21312, 21339, 21455, 21488, 21492, 21507, 21518, 21557, 21610, 21616, 21623, 21633, 21644, 21646, 21661, 21709, 21755, 21767, 21768, 21788, 21792, 21804, 21806, 21882, 21932, 21974, 21985, 22046, 22059, 22096, 22123, 22149, 22164, 22193, 22206, 22221, 22232, 22240, 22241, 22275, 22284, 22302, 22344, 22358, 22359, 22392, 22410, 22433, 22437, 22449, 22459, 22462, 22469, 22510, 22536, 22540, 22542, 22563, 22592, 22613, 22639, 22655, 22693, 22695, 22707, 22710, 22711, 22723, 22726, 22742, 22745, 22753, 22838, 22889, 22899, 22912, 22916, 23003, 23014, 23039, 23060, 23112, 23131, 23136, 23157, 23160, 23168, 23171, 23199, 23214, 23216, 23244, 23257, 23260, 23261, 23269, 23278, 23280, 23305, 23316, 23326, 23351, 23370, 23383, 23404, 23415, 23442, 23443, 23449, 23451, 23501, 23515, 23521, 23526, 23534, 23557, 23562, 23567, 23568, 23579, 23585, 23626, 23631, 23632, 23639, 23644, 23814, 23832, 23895, 23926, 23929, 23987, 23990, 24041, 24071, 24077, 24119, 24122, 24136, 24179, 24214, 24218, 24242, 24245, 24292, 24303, 24304, 24316, 24391, 24393, 24422, 24424, 24429, 24433, 24493, 24501, 24525, 24535, 24578, 24587, 24631, 24645, 24649, 24652, 24656, 24667, 24668, 24679, 24682, 24727, 24742, 24815, 24831, 24838, 24871, 24881, 24914, 24921, 24972, 24973, 24988, 25001, 25022, 25040, 25050, 25051, 25062, 25091, 25108, 25164, 25168, 25171, 25190, 25191, 25192, 25198, 25210, 25242, 25252, 25281, 25400, 25430, 25442, 25444, 25470, 25515, 25530, 25543, 25547, 25555, 25565, 25567, 25569, 25598, 25663, 25669, 25675, 25677, 25681, 25692, 25695, 25707, 25712, 25714, 25747, 25762, 25778, 25815, 25834, 25842, 25856, 25858, 25859, 25874, 25878, 25897, 25911, 25925, 25939, 25956, 25966, 25976, 26006, 26060, 26081, 26097, 26101, 26119, 26146, 26161, 26191, 26218, 26221, 26239, 26249, 26324, 26335, 26337, 26379, 26391, 26392, 26411, 26424, 26456, 26460, 26481, 26528, 26575, 26578, 26588, 26589, 26600, 26606, 26633, 26634, 26651, 26672, 26679, 26706, 26728, 26729, 26734, 26750, 26806, 26808, 26818, 26831, 26893, 26896, 26898, 26991, 26999, 27024, 27026, 27033, 27087, 27088, 27119, 27122, 27191, 27229, 27257, 27284, 27322, 27337, 27384, 27446, 27450, 27482, 27486, 27499, 27511, 27520, 27541, 27584, 27612, 27673, 27681, 27766, 27795, 27807, 27814, 27833, 27835, 27851, 27856, 27874, 27876, 27917, 27953, 27995, 28039, 28046, 28083, 28084, 28089, 28094, 28099, 28100, 28104, 28160, 28161, 28165, 28166, 28171, 28172, 28173, 28176, 28177, 28197, 28251, 28276, 28302, 28308, 28312, 28354, 28394, 28440, 28445, 28462, 28464, 28477, 28490, 28555, 28564, 28572, 28576, 28598, 28621, 28630, 28648, 28652, 28691, 28692, 28725, 28728, 28732, 28744, 28746, 28751, 28774, 28806, 28816, 28817, 28826, 28848, 28849, 28854, 28864, 28899, 28904, 28914, 28925, 28966, 28968, 29015, 29039, 29055, 29075, 29121, 29127, 29137, 29140, 29144, 29175, 29219, 29226, 29241, 29249, 29273, 29295, 29300, 29317, 29353, 29368, 29393, 29404, 29448, 29460, 29506, 29511, 29530, 29531, 29558, 29573, 29609, 29628, 29781, 29794, 29816, 29864, 29869, 29889, 29915, 29917, 29931, 29951, 29985, 30005, 30015, 30032, 30037, 30039, 30095, 30110, 30112, 30117, 30121, 30142, 30148, 30159, 30176, 30207, 30217, 30238, 30294, 30324, 30371, 30387, 30452, 30465, 30477, 30482, 30492, 30494, 30514, 30520, 30532, 30547, 30580, 30586, 30621, 30627, 30634, 30650, 30655, 30672, 30676, 30702, 30709, 30724, 30728, 30777, 30781, 30793, 30802, 30815, 30818, 30861, 30874, 30883, 30892, 30898, 31020, 31041, 31044, 31054, 31058, 31059, 31060, 31112, 31131, 31153, 31160, 31211, 31213, 31234, 31240, 31253, 31257, 31316, 31339, 31347, 31372, 31413, 31430, 31484, 31488, 31527, 31550, 31583, 31608, 31623, 31628, 31641, 31654, 31663, 31697, 31701, 31703, 31721, 31750, 31755, 31784, 31786, 31797, 31832, 31846, 31878, 31889, 31954, 31969, 31975, 31993, 32009, 32014, 32034, 32055, 32057, 32076, 32077, 32127, 32133, 32205, 32236, 32293, 32301, 32360, 32365, 32379, 32386, 32419, 32453, 32454, 32455, 32464, 32466, 32489, 32514, 32520, 32534, 32538, 32542, 32573, 32592, 32634, 32642, 32653, 32659, 32678, 32679, 32725, 32758, 32767, 32771, 32788, 32816, 32829, 32851, 32874, 32875, 32881, 32883, 32885, 32887, 32891, 32892, 32900, 32905, 32912, 32913, 32922, 32932, 32940, 32961, 33008, 33021, 33048, 33059, 33108, 33110, 33115, 33151, 33166, 33207, 33210, 33220, 33223, 33230, 33261, 33314, 33326, 33349, 33351, 33359, 33365, 33368, 33379, 33391, 33423, 33438, 33473, 33497, 33527, 33538, 33555, 33576, 33582, 33607, 33613, 33619, 33625, 33630, 33641, 33670, 33671, 33689, 33696, 33730, 33733, 33777, 33799, 33830, 33869, 33882, 33886, 33890, 33915, 33974, 34043, 34047, 34048, 34059, 34074, 34079, 34134, 34149, 34163, 34182, 34234, 34244, 34260, 34291, 34365, 34373, 34384, 34415, 34421, 34423, 34445, 34452, 34462, 34481, 34483, 34487, 34501, 34507, 34509, 34524, 34560, 34563, 34584, 34642, 34660, 34672, 34685, 34689, 34723, 34754, 34755, 34778, 34794, 34801, 34816, 34818, 34819, 34824, 34826, 34856, 34883, 34886, 34888, 34902, 34933, 34945, 34949, 35017, 35106, 35138, 35158, 35183, 35216, 35226, 35274, 35275, 35293, 35297, 35329, 35368, 35371, 35382, 35440, 35470, 35503, 35517, 35530, 35543, 35557, 35571, 35597, 35605, 35617, 35654, 35676, 35730, 35737, 35755, 35762, 35763, 35781, 35787, 35833, 35863, 35871, 35907, 35928, 35953, 35973, 36062, 36067, 36071, 36122, 36159, 36160, 36165, 36179, 36180, 36190, 36238, 36241, 36270, 36271, 36275, 36276, 36312, 36329, 36388, 36450, 36453, 36460, 36464, 36480, 36512, 36513, 36525, 36541, 36547, 36550, 36566, 36593, 36602, 36652, 36656, 36716, 36754, 36760, 36763, 36777, 36790, 36792, 36793, 36808, 36813, 36818, 36839, 36862, 36887, 36900, 36977, 36987, 37000, 37014, 37039, 37041, 37067, 37076, 37101, 37114, 37116, 37125, 37175, 37195, 37197, 37199, 37200, 37203, 37204, 37320, 37321, 37324, 37379, 37394, 37397, 37422, 37426, 37436, 37456, 37481, 37485, 37486, 37495, 37540, 37560, 37564, 37584, 37593, 37595, 37617, 37619, 37663, 37687, 37734, 37736, 37738, 37739, 37749, 37782, 37783, 37792, 37832, 37839, 37841, 37856, 37871, 37876, 37900, 37931, 37932, 37946, 37971, 38002, 38014, 38025, 38059, 38064, 38080, 38099, 38100, 38102, 38103, 38110, 38116, 38131, 38134, 38135, 38138, 38146, 38147, 38167, 38170, 38214, 38220, 38250, 38255, 38264, 38291, 38303, 38308, 38325, 38331, 38347, 38352, 38368, 38382, 38397, 38399, 38448, 38456, 38457, 38504, 38515, 38520, 38524, 38536, 38540, 38560, 38567, 38601, 38610, 38632, 38637, 38641, 38687, 38690, 38704, 38765, 38780, 38789, 38803, 38847, 38850, 38853, 38855, 38860, 38879, 38882, 38896, 38905, 38937, 39020, 39049, 39109, 39110, 39117, 39132, 39137, 39161, 39165, 39166, 39171, 39180, 39231, 39239, 39243, 39249, 39266, 39298, 39329, 39332, 39385, 39389, 39409, 39413, 39419, 39435, 39457, 39463, 39511, 39512, 39525, 39527, 39551, 39559, 39576, 39606, 39646, 39670, 39675, 39695, 39702, 39772, 39783, 39785, 39802, 39804, 39818, 39875, 39879, 39890, 39891, 39924, 39997, 40000, 40027, 40033, 40048, 40067, 40119, 40132, 40170, 40226, 40261, 40268, 40284, 40292, 40314, 40321, 40324, 40338, 40341, 40345, 40357, 40385, 40395, 40411, 40462, 40476, 40477, 40479, 40486, 40489, 40511, 40524, 40531, 40561, 40562, 40577, 40614, 40632, 40648, 40651, 40661, 40673, 40677, 40680, 40686, 40690, 40691, 40704, 40710, 40711, 40733, 40747, 40763, 40764, 40788, 40830, 40900, 40909, 40913, 40918, 40925, 40932, 40935, 40966, 40970, 40973, 40985, 40986, 41006, 41008, 41032, 41040, 41041, 41061, 41069, 41081, 41119, 41173, 41175, 41176, 41179, 41180, 41227, 41230, 41234, 41272, 41299, 41314, 41318, 41382, 41385, 41417, 41424, 41435, 41481, 41513, 41522, 41524, 41536, 41567, 41578, 41654, 41701, 41702, 41707, 41709, 41752, 41762, 41765, 41768, 41771, 41781, 41795, 41804, 41805, 41824, 41876, 41900, 41902, 41922, 41926, 41968, 42008, 42021, 42081, 42103, 42115, 42139, 42147, 42174, 42185, 42186, 42262, 42270, 42277, 42279, 42299, 42301, 42302, 42303, 42310, 42327, 42339, 42343, 42348, 42350, 42354, 42373, 42406, 42450, 42462, 42472, 42486, 42507, 42540, 42571, 42581, 42582, 42587, 42592, 42623, 42656, 42667, 42673, 42689, 42718, 42723, 42772, 42781, 42800, 42805, 42835, 42838, 42872, 42963, 42964, 42966, 42977, 43010, 43039, 43040, 43043, 43053, 43080, 43122, 43126, 43148, 43156, 43224, 43228, 43236, 43237, 43260, 43292, 43314, 43394, 43434, 43446, 43451, 43465, 43477, 43481, 43492, 43523, 43531, 43571, 43612, 43615, 43640, 43656, 43725, 43737, 43751, 43769, 43803, 43810, 43819, 43881, 43885, 43902, 43903, 43911, 43941, 43946, 43952, 49354, 49374, 49390, 49498, 49518, 49520, 49532, 49547, 49559, 49588, 49594, 49595, 49606, 49619, 49622, 49625, 49646, 49649, 49662, 49675, 49680, 49687, 49691, 49695, 49712, 49720, 49728, 49737, 49738, 49747, 49760, 49772, 49779, 49780, 49788, 49832, 49850, 49874, 49894, 49904, 49955, 49969, 49971, and 49974.

In some embodiments, a heterologous gene effector comprises, consists essentially of, or consists of an amino acid sequence that is at most about 500, at most about 450, at most about 400, at most about 350, at most about 300, at most about 250, at most about 200, at most about 190, at most about 180, at most about 170, at most about 160, at most about 150, at most about 140, at most about 130, at most about 120, at most about 110, at most about 100, at most about 90, at most about 85, at most about 80, at most about 75, at most about 70, at most about 65, at most about 60, at most about 55, at most about 50, at most about 45, at most about 40, at most about 35, at most about 30, at most about 25, or at most about 20 amino acids in length.

In some embodiments, a heterologous gene effector comprises, consists essentially of, or consists of an amino acid sequence that is at least about 10, at least about 20, at least about 30, at least about 40, at least about 50, at least about 55, at least about 60, at least about 65, at least about 70, at least about 75, at least about 80, or at least about 85 amino acids in length.

In some embodiments, a heterologous gene effector comprises, consists essentially of, or consists of an amino acid sequence that is about 20-500, about 20-400, about 20-300, about 20-250, about 20-200, about 20-150, about 20-125, about 20-100, about 20-75, about 20-50, 50-500, about 50-400, about 50-300, about 50-250, about 50-200, about 50-150, about 50-125, about 50-100, about 50-75, about 80-500, about 80-400, about 80-300, about 80-250, about 80-200, about 80-150, about 80-125, about 80-100, or about 80-90 amino acids in length. In some embodiments, a heterologous gene effector comprises, consists essentially of, or consists of an amino acid sequence that is about 85 amino acids in length.

The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length, maximizing the number of matches and minimizes the number of gaps. Default settings can be used.

In some embodiments, a heterologous gene effector comprises two or more sequences disclosed herein, for example, two, three, four, five, six, seven, eight, nine, ten, two or more, three or more, four or more, five or more, six or more, seven or more, eight or more, nine or more, or ten or more of SEQ ID NOs: 16-49333 and 49353-50052. In some embodiments, the two or more sequences originate from or are encoded by the same DNA sequence, e.g., are encoded by adjacent stretches of nucleotides from the same source gene or genome, such as before and after a putative or predicted stop codon. In some embodiments, the two or more sequences originate from or are encoded by different DNA sequences, e.g., different genes.

In some embodiments, a heterologous gene effector is not, or does not contain a sequence from, a chromatin regulator that has been previously identified. In some embodiments, a heterologous gene effector is not, or does not contain a sequence from, a chromatin regulator that has previously been targeted to regulate expression of a gene using a guide moiety as disclosed herein. In some embodiments, a heterologous gene effector is not P300, TET1, TET2, TET3, and/or HSF1, or does not contain a sequence from P300, TET1, TET2, TET3, and/or HSF1.

In some embodiments, a heterologous gene effector is not, or does not contain a sequence from, a transcriptional activator that has been previously identified. In some embodiments, a heterologous gene effector is not, or does not contain a sequence from, a transcriptional activator that has previously been targeted to regulate expression of a gene using a guide moiety as disclosed herein. In some embodiments, a heterologous gene effector is not, or does not contain a sequence from, VP64, P65, Rta, VPR, AD2, CR3, ELKF1, GATA4, PRVIE, p53, SP1, MYOD, MEF2C, TAX, PPAR-gamma, MED1, MED7, MED17, MED26, MED29, TBP, GTF2H-2D, GTF2B, CBP, HSF1, MS2-p65-HSF1, MS2-TET1, NLS-dCas9-VP64, P300, p65, PRDM9, PUFa-GADD45A-TET1, R2, SunTag-scFv-sfGFP-TET1CD, TET1, TET2, TET3, VP120, VP16, VP16, VP16, VP48, VP64, VP64 or p65+/−HSF1 or MyoD1, and/or VPR (Vp64+p65+Rta).

In some embodiments, a heterologous gene effector is not, or does not contain a sequence from, a transcriptional repressor that has been previously identified. In some embodiments, a heterologous gene effector is not, or does not contain a sequence from, a transcriptional repressor that has previously been targeted to regulate expression of a gene using a guide moiety as disclosed herein. In some embodiments, a heterologous gene effector is not, or does not contain a sequence from KRAB, Mad mSIN3 interaction domain (SID), ERF repressor domain (ERD), cat3a, last 301 amino acids of Dnmt3a Isoform 1, dCas9-KRAB-MeCP2, DNMT3A, DNMT3A, DNMT3A, DNMT3A R887E-DNMT3L, DNMT3A-DNMT3L, DNMT3B, EZH2, HDAC, KRAB-DNMT3A, KRAB-DNMT3A-DNMT3L, KRAB-DNMT3L, LSD1, M.SssI, MQ1, MQ1 Q147E, SID4×, and/or SuntTag-DNMT3A.

Heterologous gene effectors can be drawn from literature and database sources and can be targeted to include diverse protein families.

### Viral Heterologous Gene Effectors

Disclosed herein, in some embodiments, are compositions and methods that utilize heterologous gene effectors from viral sources, e.g., viral gene effectors.

Hundreds of species of virus infect humans. Gene effector domains can be encoded by viral genomes to modulate expression of host and/or viral genes. A census of viral transcriptional regulators from 20 virus families identified 419 transcriptional regulators, of which 171 were DNA-binding (Liu, Xing, et al. “Human virus transcriptional regulators.” Cell 182.1 (2020): 24-37). Further, the majority of the vast number of viral particles estimated to exist have not been characterized, but may harbor heterologous gene effectors that could be useful when incorporated into compositions and methods of the disclosure. For example, such viral heterologous gene effectors from viral sources may comprise novel effector activity, genome organization control, and/or nucleic acid packaging functions. Viruses lacking human tropism may nonetheless produce desirable activity when employed in compositions and methods of the disclosure (for example, used in engineered complexes disclosed herein). Epidemiological studies have identified zoonotic infectious viral species with verified human-tropic activity that emerge from rich evolutionary processes in key viral reservoirs, such as bats, supporting the idea that gene effector activity can be present even in viruses that lack human tropism. Thus, in some embodiments, the disclosure includes sequence data collected from metagenomic surveys.

Transcriptional regulatory activity of viral heterologous gene effectors can comprise or affect, for example, chromatin remodeling, RNA polymerase recruitment (e.g., RNA Pol II), transcription imitation, transcription elongation, DNA replication, viral transcription, nucleic acid transport, or a combination thereof. Transcriptional regulatory activity of viral heterologous gene effectors can involve, for example, direct binding to nucleic acids (e.g., E2, ICP4, Zta, IRFs), indirect binding to nucleic acids (e.g., EBNA2, ElA), regulation of transcriptional machinery or components thereof (e.g., Tat, ElA, IE2), modification of chromatin (e.g., ElA, Hbx, Tat), or a combination thereof.

Compared to human transcriptional regulators, viral transcriptional regulators can show little sequence conservation of DNA-binding domains, exhibit higher mutation rates, and be poorly structurally defined (<12%). Defining discrete viral transcriptional regulators can require reliance on species ortholog homology, and limited DNA motif knowledge can mean that defining viral transcriptional regulator binding targets requires experimental data (ChIP, EMSA, DNase footprinting, etc).

In some embodiments, a list of candidate viral heterologous gene effectors can be stratified by factors such as activation versus repression, target cellular pathway, evolutionary sequence constraint, binding of DNA/RNA/both, protein folding pattern, host/cell tropism, direct/indirect activity, binding promiscuity, nuclear/cytoplasmic action, and other criteria. In some embodiments, such stratifications allow design of a screen library of the disclosure that encompasses a broad spectrum of potential molecular functions, thereby increasing the likelihood for novel effector discovery.

In some embodiments, compositions and methods of the disclosure utilize candidate heterologous gene effectors from validated human virus transcriptional regulators. Such transcriptional regulators can be validated by, for example, ChIP/ChIP-seq, EMSA, SELEX, reporter assays, binding assays, or crystal structures. Non limiting examples of viral families that can have validated human virus transcriptional regulators include Adenoviridae, Arenaviridae, Bornaviridae, Coronaviridae, Filoviridae, Flaviviridae, Hepadnaviridae, Herpesviridae, Orthomyxoviridae, Papillomaviridae, Paramyxoviridae, Parvoviridae, Peribunyaviridae, Phenuiviridae, Pneumoviridae, Polyomaviridae, Poxviridae, Retroviridae, and Rhabdoviridae.

In some embodiments, compositions and methods of the disclosure utilize candidate heterologous gene effectors from viruses or virus families that have been shown to be capable of zoonotic transmission to humans. The viruses can be capable of infecting, for example, mammals, birds, swine, non-human primates, rodents, ungulates, reptiles, or amphibians. Non limiting examples of viral families that have been shown to be capable of zoonotic transmission to humans include Flaviviridae, Lyssaviridae, Filoviridae, Paramyxoviridae, Orthomyxoviridae, Coronaviridae, Reoviridae, Togaviridae, Phenuviridae, Hantaviridae, Bunyaviridae, and Rhabdoviridae.

In some embodiments, compositions and methods of the disclosure utilize candidate heterologous gene effectors from viruses or virus families that can infect humans and bats. In some embodiments, sequence data for the candidate heterologous gene effectors from viruses or virus families that can infect humans and bats are obtained from a database, such as dBatVir. Non limiting examples of viral families that can infect humans and bats include Flaviviridae, Lyssaviridae, Filoviridae, Paramyxoviridae, Orthomyxoviridae, Coronaviridae, Reoviridae, Togaviridae, Phenuviridae, Hantaviridae, Bunyaviridae, and Rhabdoviridae.

In some embodiments, compositions and methods of the disclosure utilize candidate heterologous gene effectors from metagenomic virus sequences. Non-limiting examples of sources of viral metagenomic data include the human gut virome, extreme environments (e.g., high-temperature and/or high-acid ecosystems which may be enriched for optimal effector qualities, including e.g. archaea-tropic viruses (e.g., sulfolobus), oceans, geothermal vents, viruses that infect non-human species and/or are from virus families that exhibit zoonotic transmission to humans, and sources that utilize structural data (e.g., X-ray, NMR) from public databases. In some embodiments, compositions and methods of the disclosure utilize candidate heterologous gene effectors from viruses that infect archaea, bacteria, cyanobacterial, algae, plants, etc., Non-limiting examples of virus species with sequence data obtained from metagenomic sources include sulfolobus, Siphoviridae, podoviridae, myoviridae, Mimiviridae, Nimaviridae, Ligamenvirales, Globuloviridae, Fuselloviridae, Bicaudaviridae, Satellite virus, Iridoviridae, Turriviridae, Caudovirales, and Phycodnaviridae.

In some embodiments, compositions and methods of the disclosure utilize candidate heterologous gene effectors from one or more viruses from the families Abyssoviridae, Ackermannviridae, Adenoviridae, Adintoviridae, Aliusviridae, Alloherpesviridae, Alphaflexiviridae, Alphasatellitidae, Alphatetraviridae, Alvernaviridae, Amalgaviridae, Amnoonviridae, Ampullaviridae, Anelloviridae, Arenaviridae, Arteriviridae, Artoviridae, Ascoviridae, Asfarviridae, Aspiviridae, Astroviridae, Atkinsviridae, Autographiviridae, Avsunviroidae, Bacilladnaviridae, Baculoviridae, Bamaviridae, Belpaoviridae, Benyviridae, Betaflexiviridae, Bicaudaviridae, Bidnaviridae, Bimaviridae, Blumeviridae, Bornaviridae, Botourmiaviridae, Bromoviridae, Caliciviridae, Carmotetraviridae, Caulimoviridae, Chaseviridae, Chrysoviridae, Chuviridae, Circoviridae, Clavaviridae, Closteroviridae, Coronaviridae, Corticoviridae, Cremegaviridae, Crepuscuviridae, Cruliviridae, Curvulaviridae, Cystoviridae, Deltaflexiviridae, Demerecviridae, Dicistroviridae, Drexlerviridae, Duinviridae, Endomaviridae, Euroniviridae, Fiersviridae, Filoviridae, Fimoviridae, Finnlakeviridae, Flaviviridae, Fuselloviridae, Gammaflexiviridae, Geminiviridae, Genomoviridae, Globuloviridae, Gresnaviridae, Guelinviridae, Guttaviridae, Halspiviridae, Hantaviridae, Hepadnaviridae, Hepeviridae, Herelleviridae, Herpesviridae, Hypoviridae, Hytrosaviridae, Iflaviridae, Inoviridae, Iridoviridae, Kitaviridae, Kolmioviridae, Lavidaviridae, Leishbuviridae, Lipothrixviridae, Lispiviridae, Malacoherpesviridae, Mamaviridae, Marseilleviridae, Matonaviridae, Matshushitaviridae, Mayoviridae, Medioniviridae, Megabimaviridae, Mesoniviridae, Metaviridae, Metaxyviridae, Microviridae, Mimiviridae, Mitoviridae, Mononiviridae, Mymonaviridae, Myoviridae, Mypoviridae, Myriaviridae, Nairoviridae, Nanghoshaviridae, Nanhypoviridae, Nanoviridae, Narnaviridae, Natareviridae, Nimaviridae, Nodaviridae, Nudiviridae, Nyamiviridae, Olifoviridae, Orthomyxoviridae, Ovaliviridae, Papillomaviridae, Paramyxoviridae, Partitiviridae, Parvoviridae, Paulinoviridae, Peribunyaviridae, Permutotetraviridae, Phasmaviridae, Phenuiviridae, Phycodnaviridae, Picobimaviridae, Picomaviridae, Plasmaviridae, Plectroviridae, Pleolipoviridae, Pneumoviridae, Podoviridae, Polycipiviridae, Polydnaviridae, Polymycoviridae, Polyomaviridae, Portogloboviridae, Pospiviroidae, Potyviridae, Poxviridae, Pseudoviridae, Qinviridae, Quadriviridae, Redondoviridae, Reoviridae, Retroviridae, Rhabdoviridae, Roniviridae, Rountreeviridae, Rudiviridae, Salasmaviridae, Sarthroviridae, Schitoviridae, Secoviridae, Simuloviridae, Sinhaliviridae, Siphoviridae, Smacoviridae, Solemoviridae, Solinviviridae, Solspiviridae, Sphaerolipoviridae, Spiraviridae, Steitzviridae, Sunviridae, Tectiviridae, Thaspiviridae, Tobaniviridae, Togaviridae, Tolecusatellitidae, Tombusviridae, Tospoviridae, Totiviridae, Tristromaviridae, Turriviridae, Tymoviridae, Virgaviridae, Wupedeviridae, Xinmoviridae, Yueviridae, Zobellviridae, or any combination thereof.

In some embodiments, compositions and methods of the disclosure utilize candidate heterologous gene effectors from one or more viruses from the subfamilies Actantavirinae, Agantavirinae, Aglimvirinae, Alphaherpesvirinae, Alphairidovirinae, Alpharhabdovirinae, Arquatrovirinae, Avulavirinae, Azeredovirinae, Bastillevirinae, Bclasvirinae, Beephvirinae, Beijerinckvirinae, Betaherpesvirinae, Betairidovirinae, Betarhabdovirinae, Braunvirinae, Brockvirinae, Bronfenbrennervirinae, Bullavirinae, Calvusvirinae, Ceronivirinae, Chebruvirinae, Chimanivirinae, Chordopoxvirinae, Cleopatravirinae, Cobavirinae, Colwellvirinae, Comovirinae, Corkvirinae, Crocarterivirinae, Crustonivirinae, Cvivirinae, Dclasvirinae, Deejayvirinae, Denniswatsonvirinae, Densovirinae, Dolichocephalovirinae, Eekayvirinae, Emmerichvirinae, Enquatrovirinae, Entomopoxvirinae, Equarterivirinae, Ermolyevavirinae, Erskinevirinae, Eucampyvirinae, Firstpapillomavirinae, Fuhrmanvirinae, Gammaherpesvirinae, Gammarhabdovirinae, Geminialphasatellitinae, Gochnauervirinae, Gofosavirinae, Gokushovirinae, Gorgonvirinae, Guemseyvirinae, Gutmannvirinae, Hamaparvovirinae, Hendrixvirinae, Heroarterivirinae, Hexponivirinae, Humphriesvirinae, Hyporhamsavirinae, Jasinkavirinae, Krylovirinae, Langleyhallvirinae, Letovirinae, Mammantavirinae, Markadamsvirinae, Mccleskeyvirinae, Mccorquodalevirinae, Mclasvirinae, Medionivirinae, Melnykvirinae, Metaparamyxovirinae, Migulavirinae, Molineuxvirinae, Mononivirinae, Nanoalphasatellitinae, Nclasvirinae, Nefertitivirinae, Northropvirinae, Nymbaxtervirinae, Okabevirinae, Okanivirinae, Orthocoronavirinae, Orthoparamyxovirinae, Orthoretrovirinae, Ounavirinae, Parvovirinae, Pclasvirinae, Peduovirinae, Petromoalphasatellitinae, Picovirinae, Piscanivirinae, Pontosvirinae, Procedovirinae, Queuovirinae, Quinvirinae, Rakietenvirinae, Regressovirinae, Remotovirinae, Repantavirinae, Reternivirinae, Rhodovirinae, Rodepovirinae, Rogunavirinae, Rothmandenesvirinae, Rubulavirinae, Sarlesvirinae, Secondpapillomavirinae, Sedoreovirinae, Sepvirinae, Serpentovirinae, Simarterivirinae, Skryabinvirinae, Slopekvirinae, Spinareovirinae, Spounavirinae, Spumaretrovirinae, Studiervirinae, Tatarstanvirinae, Tempevirinae, Tevenvirinae, Tiamatvirinae, Torovirinae, Trabyvirinae, Trivirinae, Tunavirinae, Tunicanivirinae, Twarogvirinae, Twortvirinae, Tybeckvirinae, Variarterivirinae, Vequintavirinae, Zealarterivirinae, or any combination thereof.

In some embodiments, compositions and methods of the disclosure utilize candidate heterologous gene effectors from viruses with a high degree of documented transcriptional regulator modularity, such as, for example, the poxviridae, Herpesviridae, or Adenoviridae families. In some embodiments, heterologous gene effectors from viruses with a high degree of documented transcriptional regulator modularity can be useful in combination with other gene effectors, for example, can be more likely to facilitate combinatorial or synergistic effects on gene transcription.

Without wishing to be bound by any particular theory, viral effectors may confer advantages of compact size and novel functional properties compared to gene effectors from alternate sources.

### Guide Moieties

Compositions and methods of the disclosure can utilize guide moieties to direct a heterologous gene effector to a target gene (e.g., target endogenous gene) or a target gene regulatory sequence. A guide moiety can confer an ability to recognize and specifically bind to the target gene or the target gene regulatory sequence.

A guide moiety can comprise a guide nucleic acid. A guide moiety can comprise a nuclease and a guide nucleic acid as disclosed herein. A guide moiety can comprise a nuclease or a part thereof, for example, an endonuclease, such as a heterologous endonuclease. The nuclease can be, e.g., a DNA nuclease and/or RNA nuclease, a modified nuclease that is nuclease-deficient or has reduced nuclease activity compared to a wild-type nuclease, a derivative thereof, a variant thereof, or a fragment thereof. In some embodiments, the guide moiety has minimal nuclease activity.

Any suitable nuclease, fragment or derivative thereof can be used in a guide moiety. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof; any variant thereof and any fragment thereof.

In some embodiments, the guide moiety comprises a DNA nuclease such as an engineered (e.g., programmable or targetable) DNA nuclease that is nuclease-deficient. In some embodiments, the guide moiety comprises a nuclease-null DNA binding protein derived from a DNA nuclease that does not induce transcriptional activation or repression of a target DNA sequence unless it is present in a complex with one or more heterologous gene effectors of the disclosure. In some embodiments, the guide moiety comprises a nuclease-null DNA binding protein derived from a DNA nuclease that can induce transcriptional activation or repression of a target DNA sequence (e.g., which can be altered or augmented by the presence of a heterologous gene effector of the disclosure).

In some embodiments, the guide moiety comprises an RNA nuclease such as an engineered (e.g., programmable or targetable) RNA nuclease. In some embodiments, the guide moiety comprises a nuclease-null RNA binding protein derived from an RNA nuclease that does not induce transcriptional activation or repression of a target RNA sequence unless it is present in a complex with one or more heterologous gene effectors of the disclosure. In some embodiments, the guide moiety comprises a nuclease-null RNA binding protein derived from a RNA nuclease that can induce transcriptional activation or repression of a target RNA sequence (e.g., which can be altered or augmented by the presence of a heterologous gene effector of the disclosure).

In some embodiments, the guide moiety comprises a nucleic acid-guided targeting system. In some embodiments, the guide moiety comprises a DNA-guided targeting system. In some embodiments, the guide moiety comprises an RNA-guided targeting system. A guide moiety can comprise and utilize, for example, a guide nucleic acid sequence that facilitates specific binding of a CRISPR-Cas system (e.g., a nuclease deficient form thereof, such as dCas9) to a target gene (e.g., target endogenous gene) or target gene regulatory sequence. Binding specificity can be determined by use of a guide nucleic acid, such as a single guide RNA (sgRNA) or a part thereof. In some embodiments, the use of different sgRNAs allows the compositions and methods of the disclosure to be used with (e.g., targeted to) different target genes (e.g., target endogenous genes) or target gene regulatory sequences.

Prokaryotic CRISPR-Cas (Clustered regularly interspaced short palindromic repeats-CRISPR associated) systems, for example, Class II CRISPR-Cas systems such as Cas9 and Cpfl, can be repurposed as a tool for regulation of gene expression, epigenome editing, and chromatin looping in compositions and methods of the disclosure. Nuclease-deactivated Cas (dCas) proteins complexed with heterologous gene effectors can allow for regulation of expression of target genes (e.g., target endogenous genes) adjacent to a site bound by the dCas.

In some embodiments, the guide moiety comprises a CRISPR-associated (Cas) protein or a Cas nuclease that functions in a non-naturally occurring CRISPR (Clustered Regularly Interspaced Short Palindromic Repeats)/Cas (CRISPR-associated) system. In bacteria, this system can provide adaptive immunity against foreign DNA.

In a wide variety of organisms including diverse mammals, animals, plants, microbes, and yeast, a CRISPR/Cas system (e.g., modified and/or unmodified) can be utilized as a genome engineering tool, or can be modified to direct specific binding of engineered proteins to target loci as disclosed herein. A CRISPR/Cas system can comprise a guide nucleic acid such as a guide RNA (gRNA) complexed with a Cas protein for targeted regulation of gene expression and/or activity or nucleic acid binding. An RNA-guided Cas protein (e.g., a Cas nuclease such as a Cas9 nuclease) can specifically bind a target polynucleotide (e.g., DNA) in a sequence-dependent manner. The Cas protein, if possessing nuclease activity, can cleave the DNA.

In some cases, the Cas protein is mutated and/or modified to yield a nuclease deficient protein or a protein with decreased nuclease activity relative to a wild-type Cas protein. A nuclease deficient protein can retain the ability to bind DNA, but may lack or have reduced nucleic acid cleavage activity.

In some embodiments, the guide moiety comprises a Cas protein that forms a complex with a guide nucleic acid, such as a guide RNA or a part thereof. In some embodiments, the guide moiety comprises a Cas protein that forms a complex with a single guide nucleic acid, such as a single guide RNA (sgRNA). In some embodiments, the guide moiety comprises a RNA-binding protein (RBP) optionally complexed with a guide nucleic acid, such as a guide RNA (e.g., sgRNA), which is able to form a complex with a Cas protein. In some embodiments, the guide moiety comprises a nuclease-null DNA binding protein derived from a DNA nuclease that can induce transcriptional activation or repression of a target DNA sequence. In some embodiments, the guide moiety comprises a nuclease-null RNA binding protein derived from a RNA.

A guide nucleic acid used in compositions and methods of the disclosure can be, for example, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 nucleotides.

In some embodiments, a guide nucleic acid used in compositions and methods of the disclosure is at most at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, or at most 40 nucleotides.

In some embodiments, a guide nucleic acid used in compositions and methods of the disclosure is between about 8 and about 40 nucleotides, between about 10 and about 40 nucleotides, between about 11 and about 40 nucleotides, between about 12 and about 40 nucleotides, between about 13 and about 40 nucleotides, between about 14 and about 40 nucleotides, between about 15 and about 40 nucleotides, between about 16 and about 40 nucleotides, between about 17 and about 40 nucleotides, between about 18 and about 40 nucleotides, between about 19 and about 40 nucleotides, between about 20 and about 40 nucleotides, between about 22 and about 40 nucleotides, between about 24 and about 40 nucleotides, between about 26 and about 40 nucleotides, between about 28 and about 40 nucleotides, between about 30 and about 40 nucleotides, between about 8 and about 30 nucleotides, between about 10 and about 30 nucleotides, between about 11 and about 30 nucleotides, between about 12 and about 30 nucleotides, between about 13 and about 30 nucleotides, between about 14 and about 30 nucleotides, between about 15 and about 30 nucleotides, between about 16 and about 30 nucleotides, between about 17 and about 30 nucleotides, between about 18 and about 30 nucleotides, between about 19 and about 30 nucleotides, between about 20 and about 30 nucleotides, between about 22 and about 30 nucleotides, between about 24 and about 30 nucleotides, between about 26 and about 30 nucleotides, between about 28 and about 30 nucleotides, between about 8 and about 25 nucleotides, between about 10 and about 25 nucleotides, between about 11 and about 25 nucleotides, between about 12 and about 25 nucleotides, between about 13 and about 25 nucleotides, between about 14 and about 25 nucleotides, between about 15 and about 25 nucleotides, between about 16 and about 25 nucleotides, between about 17 and about 25 nucleotides, between about 18 and about 25 nucleotides, between about 19 and about 25 nucleotides, between about 20 and about 25 nucleotides, between about 22 and about 25 nucleotides, between about 24 and about 25 nucleotides, between about 8 and about 20 nucleotides, between about 10 and about 20 nucleotides, between about 11 and about 20 nucleotides, between about 12 and about 20 nucleotides, between about 13 and about 20 nucleotides, between about 14 and about 20 nucleotides, between about 15 and about 20 nucleotides, between about 16 and about 20 nucleotides, between about 17 and about 20 nucleotides, between about 18 and about 20 nucleotides, between about 19 and about 20 nucleotides, between about 8 and about 18 nucleotides, between about 10 and about 18 nucleotides, between about 11 and about 18 nucleotides, between about 12 and about 18 nucleotides, between about 13 and about 18 nucleotides, between about 14 and about 18 nucleotides, between about 15 and about 18 nucleotides, between about 16 and about 18 nucleotides, between about 8 and about 16 nucleotides, between about 10 and about 16 nucleotides, between about 11 and about 16 nucleotides, between about 12 and about 16 nucleotides, between about 13 and about 16 nucleotides, between about 14 and about 16 nucleotides, or between about 15 and about 16 nucleotides.

A guide nucleic acid can be a guide RNA or a part thereof.

Any suitable CRISPR/Cas system can be used. A CRISPR/Cas system can be referred to using a variety of naming systems. A CRISPR/Cas system can be a type I, a type II, a type III, a type IV, a type V, a type VI system, or any other suitable CRISPR/Cas system. A CRISPR/Cas system as used herein can be a Class 1, Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpfl, C2cl, C2c2, C2c3 or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g.,1II, IIIA, IIIB, IIIC, 1ID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g.,1I, IIA, IIB) and type V CRISPR/Cas type. CRISPR systems can be complementary to each other, and/or can lend functional units in trans to facilitate CRISPR locus targeting.

A guide moiety disclosed herein can comprise a nuclease, for instance, a heterologous nuclease (e.g., a Cas protein that is operatively coupled to a heterologous gene effector). The nuclease can have a length that is less than a threshold length. The threshold length can be at most about 1,000 amino acids, at most about 950 amino acids, at most about 900 amino acids, at most about 850 amino acids, at most about 800 amino acids, at most about 750 amino acids, at most about 700 amino acids, at most about 650 amino acids, at most about 600 amino acids, at most about 550 amino acids, at most about 500 amino acids, at most about 450 amino acids, at most about 400 amino acids, at most about 350 amino acids, or at most about 300 amino acids. The threshold length can be at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, at least about 850 amino acids, at least about 900 amino acids, at least about 950 amino acids, or at least about 1,000 amino acids.

When a guide moiety comprises a Cas protein or derivative thereof, the Cas protein or derivative thereof can be a Class 1 or a Class 2 Cas protein. A Cas protein can be a type I, type II, type III, type IV, type V Cas protein, or type VI Cas protein. A Cas protein can comprise one or more domains. Non-limiting examples of domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein or fragment thereof that is fused to other proteins or polypeptides. A Cas protein can be a chimera of various Cas proteins, for example, comprising domains from different Cas proteins.

Non-limiting examples of Cas proteins include c2cl, C2c2, c2c3, Cas1, Cas1B, Cas2, Cas3, Cas4, Cas5, Cas5e (CasD), Cash, Cas6e, Cas6f, Cas7, Cas8a, Cas8al, Cas8a2, Cas8b, Cas8c, Cas9 (Csn1 or Csx12), CasIO, Cas10d, CasIO, Cas12a, Cas10d, CasF, CasG, CasH, Cpfl, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cul966, Cas13a, Cas13b, Cas13c, Cas13d, Cas13X, Cas13Y, and homologs or modified versions thereof.

In some cases, the Cas protein as disclosed herein may not and need not be Cas9 or Cas12a. The Cas protein as disclosed herein can have a smaller size as compared to Cas9 or Cas12a. The Cas protein as disclosed herein can be derived from Un1Cas12fl (or Cas14a1). For example, the Cas protein as disclosed herein can comprise an amino acid sequence that is at least about 50%, at least about 60%, at least about 70%, at least about 75% at least about 80%, at least about 85%, at least about 90%, at least about 95%, at least about 96%, at least about 97%, at least about 98%, at least about 99%, or substantially about 100% identical to the polypeptide sequence of SEQ ID NO: 49342. In another example, the Cas protein as disclosed herein can comprise an amino acid sequence that is at least about 50%, at least about 60%, at least about 70%, at least about 75% at least about 80%, at least about 85%, at least about 90%, at least about 95%, at least about 96%, at least about 97%, at least about 98%, at least about 99%, or substantially about 100% identical to the polypeptide sequence of SEQ ID NO: 49343. As disclosed herein, SEQ ID NO: 49342 encodes the polypeptide sequence of Un1Cas12fl (or Cas14a1). As disclosed herein, SEQ ID NO: 49343 encodes an engineered variant of Un1Cas12fl with reduced nuclease activity. In some embodiments, Un1Cas12fl or a derivative thereof, such as an engineered variant of Un1Cas12fl with reduced nuclease activity, can be referred to as CasMini or dCasMini.

TABLE 1
illustrative Cas sequences

| SEQ ID | | |
|---|---|---|
| NO: | Description | Sequence |
| 49342 | Un1Cas12f1 | MAKNTITKTLKLRIVRPYNSAEVEKIVADEKNNREKIALEKNK |
| | | DKVKEACSKHLKVAAYCTTQVERNACLFCKARKLDDKFYQK |
| | | LRGQFPDAVFWQEISEIFRQLQKQAAEIYNQSLIELYYEIFIKGK |
| | | GIANASSVEHYLSDVCYTRAAELFKNAAIASGLRSKIKSNFRLK |
| | | ELKNMKSGLPTTKSDNFPIPLVKQKGGQYTGFEISNHNSDFIIKI |
| | | PFGRWQVKKEIDKYRPWEKFDFEQVQKSPKPISLLLSTQRRKR |
| | | NKGWSKDEGTEAEIKKVMNGDYQTSYIEVKRGSKIGEKSAW |
| | | MLNLSIDVPKIDKGVDPSIIGGIDVGVKSPLVCAINNAFSRYSIS |
| | | DNDLFHFNKKMFARRRILLKKNRHKRAGHGAKNKLKPITILTE |
| | | KSERFRKKLIERWACEIADFFIKNKVGTVQMENLESMKRKEDS |
| | | YFNIRLRGFWPYAEMQNKIEFKLKQYGIEIRKVAPNNTSKTCS |
| | | KCGHLNNYFNFEYRKKNKFPHFKCEKCNFKENADYNAALNIS |
| | | NPKLKSTKEEP |
| 49343 | deactivated | MAKNTITKTLKLRIVRPYNSAEVEKIVADEKNNREKIALEKNK |
| | nuclease | DKVKEACSKHLKVAAYCTTQVERNACLFCKARKLDDKFYQK |
| | variant of | LRGQFPDAVFWQEISEIFRQLQKQAAEIYNQSLIELYYEIFIKGK |
| | Un1Cas12f1 | GIANASSVEHYLSRVCYRRAAELFKNAAIASGLRSKIKSNFRLK |
| | | ELKNMKSGLPTTKSDNFPIPLVKQKGGQYTGFEISNHNSDFIIKI |
| | | PFGRWQVKKEIDKYRPWEKFDFEQVQKSPKPISLLLSTQRRKR |
| | | NKGWSKDEGTEAEIKKVMNGDYQTSYIEVKRGSKICEKSAW |
| | | MLNLSIDVPKIDKGVDPSIIGGIAVGVRSPLVCAINNAFSRYSIS |
| | | DNDLFHFNKKMFARRRILLKKNRHKRAGHGAKNKLKPITILTE |
| | | KSERFRKKLIERWACEIADFFIKNKVGTVQMENLESMKRKEDS |
| | | YFNIRLRGFWPYAEMQNKIEFKLKQYGIEIRKVAPNNTSKTCS |
| | | KCGHLNNYFNFEYRKKNKFPHFKCEKCNFKENAAYNAALNIS |
| | | NPKLKSTKERP |

A Cas protein or fragment or derivative thereof can be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas nap hthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractorsalsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinellasuccinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

A Cas protein as used herein can be a wildtype or a modified form of a Cas protein. A Cas protein can be an active variant, inactive variant, or fragment of a wild type or modified Cas protein. A Cas protein can comprise an amino acid change such as a deletion, insertion, substitution, variant, mutation, fusion, chimera, or any combination thereof relative to a wild-type version of the Cas protein. A Cas protein can be a polypeptide with at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a wild type Cas protein. A Cas protein can be a polypeptide with at most about 5%, at most about 10%, at most about 20%, at most about 30%, at most about 40%, at most about 50%, at most about 60%, at most about 70%, at most about 80%, at most about 90%, or at most about 100% sequence identity and/or sequence similarity to a wild type exemplary Cas protein. Variants or fragments can comprise at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a wild type or modified Cas protein or a portion thereof. Variants or fragments can be targeted to a nucleic acid locus in complex with a guide nucleic acid while lacking nucleic acid cleavage activity.

A Cas protein can comprise one or more nuclease domains, such as DNase domains. For example, a Cas9 protein can comprise a RuvC-like nuclease domain and/or an HNH-like 20 nuclease domain. The in a nuclease active form of Cas9, RuvC and HNH domains can each cut a different strand of double-stranded DNA to make a double-stranded break in the DNA. A Cas protein can comprise only one nuclease domain (e.g., Cpfl comprises RuvC domain but lacks HNH domain). In some embodiments, nuclease domains are absent. In some embodiments, nuclease domains are present but inactive or have reduced or minimal activity. In some embodiments, nuclease domains are present and active.

One or a plurality of the nuclease domains (e.g., RuvC, HNH) of a Cas protein can be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. If all of the nuclease domains of a Cas protein (e.g., both RuvC and HNH nuclease domains in a Cas9 protein; RuvC nuclease domain in a Cpfl protein) are deleted or mutated, the resulting Cas protein can have a reduced or no ability to cleave both strands of a double-stranded DNA. An example of a mutation that can convert a Cas9 protein into a nickase is a D10A (aspartate to alanine at position 10 of Cas9) mutation in the RuvC domain of Cas9 from S. pyogenes. H939A (histidine to alanine at amino acid position 839) or H840A (histidine to alanine at amino acid position 840) in the HNH domain of Cas9 from S. pyogenes can convert the Cas9 into a nickase. An example of a mutation that can convert a Cas9 protein into a dead Cas9 is a D10A (aspartate to alanine at position 10 of Cas9) mutation in the RuvC domain and H939A (histidine to alanine at amino acid position 839) or H840A (histidine to alanine at amino acid position 840) in the HNH domain of Cas9 from S. pyogenes.

A nuclease dead Cas protein can comprise one or more mutations relative to a wild-type version of the protein. The mutation can result in no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity in one or more of the plurality of nucleic acid-cleaving domains of the wild-type Cas protein. The mutation can result in one or more of the plurality of nucleic acid-cleaving domains retaining the ability to cleave the complementary strand of the target nucleic acid but reducing its ability to cleave the non-complementary strand of the target nucleic acid. The mutation can result in one or more of the plurality of nucleic acid-cleaving domains retaining the ability to cleave the non-complementary strand of the target nucleic acid but reducing its ability to cleave the complementary strand of the target nucleic acid. The mutation can result in one or more of the plurality of nucleic acid-cleaving domains lacking the ability to cleave the complementary strand and the non-complementary strand of the target nucleic acid. The residues to be mutated in a nuclease domain can correspond to one or more catalytic residues of the nuclease. For example, residues in the wild type exemplary S. pyogenes Cas9 polypeptide such as Asp10, His840, Asn854 and Asn856 can be mutated to inactivate one or more of the plurality of nucleic acid-cleaving domains (e.g., nuclease domains). The residues to be mutated in a nuclease domain of a Cas protein can correspond to residues Asp10, His840, Asn854 and Asn856 in the wild type S. pyogenes Cas9 polypeptide, for example, as determined by sequence and/or structural alignment.

As non-limiting examples, residues D10, G12, G17, E762, H840, N854, N863, H982, H983, A984, D986, and/or A987 (or the corresponding mutations of any of the Cas proteins) can be mutated. For example, e.g., D 10A, G12A, G17A, E762A, H840A, N854A, N863A, H982A, H983A, A984A, and/or D986A. Mutations other than alanine substitutions can be suitable.

A D10A mutation can be combined with one or more of H840A, N854A, or N856A mutations to produce a Cas9 protein substantially lacking DNA cleavage activity (e.g., a dead Cas9 protein). A H840A mutation can be combined with one or more of D10A, N854A, or N856A mutations to produce a site-directed polypeptide substantially lacking DNA cleavage activity. An N854A mutation can be combined with one or more of H840A, D10A, or N856A mutations to produce a site-directed polypeptide substantially lacking DNA cleavage activity. A N856A mutation can be combined with one or more of H840A, N854A, or D10A mutations to produce a site-directed polypeptide substantially lacking DNA cleavage activity.

In some embodiments, a Cas protein is a Class 2 Cas protein. In some embodiments, a Cas protein is a type II Cas protein. In some embodiments, the Cas protein is a Cas9 protein, a modified version of a Cas9 protein, or derived from a Cas9 protein. For example, a Cas9 protein lacking cleavage activity. In some embodiments, the Cas9 protein is a Cas9 protein from S. pyogenes (e.g., SwissProt accession number Q99ZW2). In some embodiments, the Cas9 protein is a Cas9 from S. aureus (e.g., SwissProt accession number J7RUA5). In some embodiments, the Cas9 protein is a modified version of a Cas9 protein from S. pyogenes or S. Aureus. In some embodiments, the Cas9 protein is derived from a Cas9 protein from S. pyogenes or S. Aureus. For example, a S. pyogenes or S. Aureus Cas9 protein lacking cleavage activity.

In some embodiments, Cas9 can generally refer to a polypeptide with at least about 5%, at least about 10%, at least about 20%, at least about 30%, at least about 40%, at least about 50%, at least about 60%, at least about 70%, at least about 80%, at least about 90%, or about 100% sequence identity and/or sequence similarity to a wild type exemplary Cas9 polypeptide (e.g., Cas9 from S. pyogenes). In some embodiments, Cas9 can refer to a polypeptide with at most about 5%, at most about 10%, at most about 20%, at most about 30%, at most about 40%, at most about 50%, at most about 60%, at most about 70%, at most about 80%, at most about 90%, or about 100% sequence identity and/or sequence similarity to a wild type Cas9 polypeptide (e.g., from S. pyogenes). Cas9 can refer to the wildtype or a modified form of the Cas9 protein that can comprise an amino acid change such as a deletion, insertion, substitution, variant, mutation, fusion, chimera, or any combination thereof.

A Cas protein can comprise an amino acid sequence having at least about 5%, at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, or 100% sequence identity or sequence similarity to a nuclease domain (e.g., RuvC domain, HNH domain) of a wild-type Cas protein.

A Cas protein, variant or derivative thereof can be modified to enhance regulation of gene expression by compositions and methods of the disclosure, e.g., as part of a complex disclosed herein. A Cas protein can be modified to increase or decrease nucleic acid binding affinity, nucleic acid binding specificity, enzymatic activity, and/or binding to other factors, such as heterodimerization or oligomerization domains and induce ligands. Cas proteins can also be modified to change any other activity or property of the protein, such as stability. For example, one or more nuclease domains of the Cas protein can be modified, deleted, or inactivated, or a Cas protein can be truncated to remove domains that are not essential for the desired function of the protein or complex. A Cas protein can be modified to modulate (e.g., enhance or reduce) the activity of the Cas protein for regulating gene expression by a complex of the disclosure that comprises a heterologous gene effector.

For example, a Cas protein can be coupled (e.g., fused, covalently coupled, or non-covalently coupled) to a heterologous gene effector (e.g., an epigenetic modification domain, a transcriptional activation domain, and/or a transcriptional repressor domain). A Cas protein can be coupled (e.g., fused, covalently coupled, or non-covalently coupled) to an oligomerization or dimerization domain as disclosed herein (e.g., a heterodimerization domain). A Cas protein can be coupled (e.g., fused, covalently coupled, or non-covalently coupled) to a heterologous polypeptide that provides increased or decreased stability. A Cas protein can be coupled (e.g., fused, covalently coupled, or non-covalently coupled) to a sequence that can facilitate degradation of the Cas protein or a complex containing the Cas protein, for example, a degron, such as an inducible degron (e.g., auxin inducible).

A Cas protein can be coupled (e.g., fused, covalently coupled, or non-covalently coupled) to any suitable number of partners, for example, at least one, at least two, at least three, at least four, or at least five, at least six, at least seven, or at least 8 partners. In some embodiments, a Cas protein of the disclosure is coupled (e.g., fused, covalently coupled, or non-covalently coupled) to at most two, at most three, at most four, at most five, at most six, at most seven, at most eight, or at most ten partners. In some embodiments, a Cas protein of the disclosure is coupled (e.g., fused, covalently coupled, or non-covalently coupled) to 1-5, 1-4, 1-3, 1-2, 2-5, 2-4, 2-3, 3-5, 3-4, or 4-5 partners. In some embodiments, a Cas protein of the disclosure is coupled (e.g., fused, covalently coupled, or non-covalently coupled) to one partner. In some embodiments, a Cas protein of the disclosure is coupled (e.g., fused, covalently coupled, or non-covalently coupled) to two partners. In some embodiments, a Cas protein of the disclosure is coupled (e.g., fused, covalently coupled, or non-covalently coupled) to three partners. In some embodiments, a Cas protein of the disclosure is coupled (e.g., fused, covalently coupled, or non-covalently coupled) to four partners. In some embodiments, a Cas protein of the disclosure is coupled (e.g., fused, covalently coupled, or non-covalently coupled) to five partners. In some embodiments, a Cas protein of the disclosure is coupled (e.g., fused, covalently coupled, or non-covalently coupled) to six partners.

A Cas protein can be a fusion protein. The fused domain or heterologous polypeptide (e.g., heterologous gene effector) can be located at the N-terminus, the C-terminus, or internally within the Cas protein.

A Cas protein can be provided in any form. For example, a Cas protein can be provided in the form of a protein, such as a Cas protein alone or complexed with a guide nucleic acid as a ribonucleoprotein. A Cas protein can be provided in a complex, for example, complexed with a guide nucleic acid and/or one or more heterologous gene effectors of the disclosure. A Cas protein can be provided in the form of a nucleic acid encoding the Cas protein, such as an RNA (e.g., messenger RNA (mRNA)), or DNA. The nucleic acid encoding the Cas protein can be codon optimized for efficient translation into protein in a particular cell or organism.

Nucleic acids encoding Cas proteins, fragments, or derivatives thereof can be stably integrated in the genome of a cell. Nucleic acids encoding Cas proteins can be operably linked to a promoter, for example, a promoter that is constitutively or inducibly active in the cell. Nucleic acids encoding Cas proteins can be operably linked to a promoter in an expression construct. Expression constructs can include any nucleic acid constructs capable of directing expression of a gene or other nucleic acid sequence of interest (e.g., a Cas gene) and which can transfer such a nucleic acid sequence of interest to a target cell.

In some embodiments, a Cas protein, variant or derivative thereof is a nuclease dead Cas (dCas) protein. A dead Cas protein can be a protein that lacks nucleic acid cleavage activity.

A Cas protein can comprise a modified form of a wild type Cas protein. The modified form of the wild type Cas protein can comprise an amino acid change (e.g., deletion, insertion, or substitution) that reduces the nucleic acid-cleaving activity of the Cas protein. For example, the modified form of the Cas protein can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive, “deactivated” and/or “dead” (abbreviated by “d”). A dead Cas protein (e.g., dCas, dCas9) can bind to a target polynucleotide but may not cleave or minimally cleaves the target polynucleotide. In some aspects, a dead Cas protein is a dead Cas9 protein.

A dCas9 polypeptide can associate with a single guide RNA (sgRNA) to activate or repress transcription of a target gene (e.g., target endogenous gene), for example, in combination with heterologous gene effector(s) disclosed herein. sgRNAs can be introduced into cells expressing the Cas or guide moiety component of the disclosure. In some cases, such cells can contain one or more different sgRNAs that target the same target gene (e.g., target endogenous gene) or target gene regulatory sequence. In other cases, the sgRNAs target different nucleic acids in the cell (e.g., different target genes, different target gene regulatory sequences, or different sequences within the same target gene or target gene regulatory sequence).

Enzymatically inactive can refer to a nuclease that can bind to a nucleic acid sequence in a polynucleotide in a sequence-specific manner, but will not cleave a target polynucleotide or will cleave it at a substantially reduced frequency. An enzymatically inactive guide moiety can comprise an enzymatically inactive domain (e.g. nuclease domain). Enzymatically inactive can refer to no activity. Enzymatically inactive can refer to substantially no activity. Enzymatically inactive can refer to essentially no activity. Enzymatically inactive can refer to an activity no more than 1%, no more than 2%, no more than 3%, no more than 4%, no more than 5%, no more than 6%, no more than 7%, no more than 8%, no more than 9%, or no more than 10% activity compared to a comparable wild-type activity (e.g., nucleic acid cleaving activity, wild-type Cas9 activity).

In some embodiments, the guide moiety does not contain a nucleic acid-guided targeting system. For example, guide moieties can include proteins that bind to a target gene (e.g., target endogenous gene) or target gene regulatory sequence based on protein structural features, such as certain nucleases disclosed herein.

In some embodiments, a guide moiety comprises a zinc finger nuclease (ZFN) or a variant, fragment, or derivative thereof. ZFN can refer to a fusion between a cleavage domain, such as a cleavage domain of Fokl, and at least one zinc finger motif (e.g., at least 2, at least 3, at least 4, or at least 5 zinc finger motifs) which can bind polynucleotides such as DNA and RNA. In some embodiments, a ZFN is used in a targeting moiety of the disclosure to bind a polynucleotide (e.g., target gene or target gene regulatory sequence), but the ZFN does not cleave or substantially does not cleave the polynucleotide, e.g., a nuclease dead ZFN. A ZFN or a variant, fragment, or derivative thereof can be fused to or associated with one of more heterologous gene effectors to form a complex of the disclosure.

The heterodimerization at certain positions in a polynucleotide of two individual ZFNs in certain orientation and spacing can lead to cleavage of the polynucleotide in nuclease-active ZFN. For example, a ZFN binding to DNA can induce a double-strand break in the DNA. In order to allow two cleavage domains to dimerize and cleave DNA, two individual ZFNs can bind opposite strands of DNA with their C-termini at a certain distance apart. In some cases, linker sequences between the zinc finger domain and the cleavage domain can require the 5′ edge of each binding site to be separated by about 5-7 base pairs. In some cases, a cleavage domain is fused to the C-terminus of each zinc finger domain.

In some embodiments, the cleavage domain of a guide moiety comprising a ZFN comprises a modified form of a wild type cleavage domain. The modified form of the cleavage domain can comprise an amino acid change (e.g., deletion, insertion, or substitution) that reduces the nucleic acid-cleaving activity of the cleavage domain. For example, the modified form of the cleavage domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the corresponding wild-type cleavage domain. The modified form of the cleavage domain can have no substantial nucleic acid-cleaving activity. In some embodiments, the cleavage domain is enzymatically inactive.

In some embodiments, a guide moiety comprises a “TALEN” or “TAL-effector nuclease” or a variant, fragment, or derivative thereof. TALENs refer to engineered transcription activator-like effector nucleases that generally contain a central domain of DNA-binding tandem repeats and a cleavage domain. TALENs can be produced by fusing a TAL effector DNA binding domain to a DNA cleavage domain. In some cases, a DNA-binding tandem repeat comprises 33-35 amino acids in length and contains two hypervariable amino acid residues at positions 12 and 13 that can recognize at least one specific DNA base pair. A transcription activator-like effector (TALE) protein can be fused to a nuclease such as a wild-type or mutated Fok1 endonuclease or the catalytic domain of Fokl. In some embodiments, a TALEN is used in a targeting moiety of the disclosure to bind a polynucleotide (e.g., target gene or target gene regulatory sequence), but the TALEN does not cleave or substantially does not cleave the polynucleotide, e.g., a nuclease dead TALEN. A TALEN or a variant, fragment, or derivative thereof can be fused to or associated with one of more heterologous gene effectors to form a complex of the disclosure.

In some embodiments, a TALEN is engineered for reduced nuclease activity. In some embodiments, the nuclease domain of a TALEN comprises a modified form of a wild type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive. A TALEN or a variant, fragment, or derivative thereof can be fused to or associated with one of more heterologous gene effectors to form a complex of the disclosure.

Several mutations to Fok1 have been made for its use in TALENs, which, for example, improve cleavage specificity or activity. Such TALENs can be engineered to bind any desired DNA sequence. TALENs can be used to generate gene modifications (e.g., nucleic acid sequence editing) by creating a double-strand break in a target DNA sequence, which in turn, undergoes NHEJ or HDR.

A TALE or a variant, fragment, or derivative thereof can be fused to or associated with one of more heterologous gene effectors to form a complex of the disclosure. In some embodiments, the transcription activator-like effector (TALE) protein is fused to a heterologous gene effector and does not comprise a nuclease. In some embodiments, a TALEN does not cleave or substantially does not cleave the polynucleotide, e.g., a nuclease dead TALE. A TALE or a variant, fragment, or derivative thereof can be fused to or associated with one of more heterologous gene effectors to form a complex of the disclosure.

In some embodiments, the complex of the transcription activator-like effector (TALE) protein and the heterologous gene effector is designed to function as a transcriptional activator. In some embodiments, the complex of the transcription activator-like effector (TALE) protein and the heterologous gene effector is designed to function as a transcriptional repressor. For example, the DNA-binding domain of the transcription activator-like effector (TALE) protein can be fused (e.g., linked) to one or more heterologous gene effectors that comprise transcriptional activation domains, or to one or more heterologous gene effectors that comprise transcriptional repression domains.

In some embodiments, a guide moiety comprises a meganuclease. Meganucleases generally refer to rare-cutting endonucleases or homing endonucleases that can be highly sequence specific. Meganucleases can recognize DNA target sites ranging from at least 12 base pairs in length, e.g., from 12 to 40 base pairs, 12 to 50 base pairs, or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. A nuclease-active meganuclease can generate a double-stranded break. In some embodiments, a meganuclease is used in a targeting moiety of the disclosure to bind a polynucleotide (e.g., target gene or target gene regulatory sequence), but the meganuclease does not cleave or substantially does not cleave the polynucleotide, e.g., a nuclease dead meganuclease. A meganuclease or a variant, fragment, or derivative thereof can be fused to or associated with one of more heterologous gene effectors to form a complex of the disclosure.

The meganuclease can be monomeric or dimeric. In some embodiments, the meganuclease is naturally-occurring (found in nature) or wild-type, and in other instances, the meganuclease is non-natural, artificial, engineered, synthetic, rationally designed, or man-made. In some embodiments, the meganuclease of the present disclosure includes an I-CreI meganuclease, I-CeuI meganuclease, I-Msol meganuclease, I-SceI meganuclease, variants thereof, derivatives thereof, and fragments thereof.

In some embodiments, the nuclease domain of a meganuclease comprises a modified form of a wild type nuclease domain. The modified form of the nuclease domain can comprise an amino acid change (e.g., deletion, insertion, or substitution) that reduces or eliminates the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive. In some embodiments, a meganuclease can bind DNA but cannot cleave the DNA. In some embodiments, a nuclease-inactive meganuclease is fused to or associated with one or more heterologous gene effectors to generate a complex of the disclosure.

In some embodiments, the guide moiety can regulate expression and/or activity of a target gene (e.g., target endogenous gene). In some embodiments, the guide moiety can edit the sequence of a nucleic acid (e.g., a gene and/or gene product). A nuclease-active Cas protein can edit a nucleic acid sequence by generating a double-stranded break or single-stranded break in a target polynucleotide.

In some embodiments, a guide moiety comprising a nuclease can generate a double-strand break in a target polynucleotide, such as DNA. A double-strand break in DNA can result in DNA break repair which allows for the introduction of gene modification(s) (e.g., nucleic acid editing). In some embodiments, a nuclease induces site-specific single-strand DNA breaks or nicks, thus resulting in HDR.

A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA can be provided.

In some embodiments, a guide moiety or complex comprising a nuclease does not generate a double-strand break in a target polynucleotide, such as DNA.

### Complexes

Disclosed herein, in some aspects, are complexes that comprise a heterologous gene effector and a guide moiety, for example, a guide nucleic acid and/or a nuclease, such as an endonuclease that lacks or substantially lacks cleavage activity.

Complexes of the disclosure can be useful, for example, for bringing one or more heterologous gene effectors into close proximity with a target gene (e.g., target endogenous gene) or target gene regulatory sequence, thereby facilitating modulation of an expression or activity level of the target gene.

In some embodiments, a complex of the disclosure binds to DNA, e.g., genomic DNA. In some embodiments, a complex of the disclosure binds to RNA, e.g., mRNA, microRNA, siRNA, or non-coding RNA. In some embodiments, a complex of the disclosure binds to DNA and RNA.

In some embodiments, a complex can modulate (e.g., increase or decrease) expression and/or activity of a target gene (e.g., target endogenous gene) by physical obstruction of a polynucleotide sequence (e.g., a promoter, enhancer, repressor, operator, or silencer, insulator, cis-regulatory element, trans-regulatory element, epigenetic modification (e.g., DNA methylation) site, coding sequence).

In some embodiments, a complex can modulate (e.g., increase or decrease) expression and/or activity of a target gene (e.g., target endogenous gene) by recruitment of additional factors effective to suppress or enhance expression of the target gene.

In some embodiments, complexes of the disclosure are used for introducing epigenetic modifications to a target gene (e.g., target endogenous gene) or target gene regulatory sequence (e.g., promoter, enhancer, silencer, insulator, cis-regulatory element, trans-regulatory element, or epigenetic modification (e.g., DNA methylation) site). In some embodiments, complexes of the disclosure are used for producing three-dimensional structures, topologically associating domains, or genomic boundaries comprising a target gene or target gene regulatory sequence (e.g., distal or proximal gene from the target gene).

In some cases, regulation of a target gene (e.g., a target endogenous gene) by a complex as disclosed herein, such as a complex comprising one or more heterologous gene effectors and a guide nucleic acid, may utilize an endogenous target gene regulatory sequence (e.g., promoter, enhancer, repressor, silencer, insulator, cis-regulatory element, trans-regulatory element, epigenetic modification (e.g., DNA methylation) site, etc.) operatively coupled to the target gene. Thus, such regulation of the target gene by the complex may not and need not involve an exogenous, synthetic, and/or heterologous regulatory sequence, such as a promoter, enhancer, repressor, silencer, insulator, cis-regulatory element, trans-regulatory element, epigenetic modification (e.g., DNA methylation) site, etc. that is heterologous with respect to the subject or the host cell. In some embodiments, regulation of the target gene by the complex does not involve use of an engineered inducible system, repressible system, and/or reporter system. In some embodiments, regulation of the target gene by the complex does not involve use of an exogenous, engineered, or synthetic regulatory element, for example, does not involve a response element that is modulated by tetracycline or analogs thereof. In some embodiments, regulation of the target gene by the complex does not involve use of a transactivator or reverse transactivator that functions as part of an engineered inducible system, repressible system, and/or reporter system. In some embodiments, regulation of the target gene by the complex does not involve a Tet off or tTA-dependent system, or a component thereof. In some embodiments, regulation of the target gene by the complex does not involve a Tet On or rtTA-dependent system, or a component thereof.

In some cases, a complex disclosed herein (e.g., a complex comprising one or more heterologous gene effectors and a guide nucleic acid) may be capable of regulating a target gene (e.g., a target endogenous gene) without any further control by a modulating agent, such as an agent that directly or indirectly allows the complex to increase or reduce expression of the target gene. In some embodiments, the complex is capable of regulating the target gene without involvement of a transactivating agent, a reverse transactivating agent, a small molecule, a drug, a chemical inducer of dimerization or multimerization, an additional inducing agent, an additional repressing agent, or any combination thereof. For example, upon introducing the individual complex to the host cell (e.g., expressing and/or transfecting each individual component of the individual complex to the host cell), such introduction may be sufficient to allow the individual complex to regulate expression or activity of the target gene.

In some embodiments, a complex comprises a heterologous gene effector and a guide moiety. In some embodiments, a complex comprises one heterologous gene effector and one guide moiety. In some embodiments, a complex comprises two heterologous gene effectors and one guide moiety. In some embodiments, a complex comprises three or more heterologous gene effectors and one guide moiety.

In some embodiments, a complex comprises a heterologous gene effector and a guide nucleic acid. In some embodiments, a complex comprises one heterologous gene effector and one guide nucleic acid. In some embodiments, a complex comprises two heterologous gene effectors and one guide nucleic acid. In some embodiments, a complex comprises three or more heterologous gene effectors and one guide nucleic acid.

In some embodiments, a complex comprises a heterologous gene effector and a nuclease (e.g., a guide moiety that comprises a nuclease, such as a heterologous endonuclease). A combination of the nuclease and the heterologous gene effector can be a chimeric fusion polypeptide comprising the nuclease and the heterologous gene effector. In some embodiments, the nuclease and the heterologous gene effector are not part of a chimeric fusion polypeptide, e.g., are not present in the same polypeptide chain. The combination of the nuclease and the heterologous gene effector can have a length that is less than a threshold length. In some embodiments, the combined length of the heterologous gene effector and the nuclease is at most about 1,200 amino acids, at most about 1,100 amino acids, at most about 1,000 amino acids, at most about 950 amino acids, at most about 900 amino acids, at most about 850 amino acids, at most about 800 amino acids, at most about 750 amino acids, at most about 700 amino acids, at most about 650 amino acids, at most about 600 amino acids, at most about 550 amino acids, at most about 500 amino acids, at most about 450 amino acids, at most about 400 amino acids, at most about 350 amino acids, or at most about 300 amino acids. In some embodiments, the combined length of the heterologous gene effector and the nuclease is at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, at least about 850 amino acids, at least about 900 amino acids, at least about 950 amino acids, at least about 1,000 amino acids, at least about 1,100 amino acids, or at least about 1,200 amino acids.

A complex disclosed herein can comprise (i) a guide moiety (for example, a nuclease or part thereof, such as a nuclease deactivated Cas, e.g., a nuclease deactivated Cas9, Cas12a, or Un1Cas12f1, optionally with a guide nucleic acid sequence), and (ii) a heterologous gene effector, wherein the heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052, e.g., over the entire length.

In some embodiments, a complex disclosed herein comprises (i) a guide moiety (for example, a guide nucleic acid sequence and/or a nuclease or part thereof, such as a nuclease deactivated Cas, e.g., a nuclease deactivated Cas9, Cas12a, or Un1Cas12fl), and (ii) a heterologous gene effector, wherein the heterologous gene effector comprises, consists essentially of, or consists of a peptide sequence with at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 97.5%, at least about 98%, at least about 98.5%, at least about 99%, at least about 99.5%, at least about 99.9%, or about 100% sequence identity or sequence similarity to any one of SEQ ID NOs: 1102, 2057, 5543, 9066, 11948, 15646, 17629, 19860, 21015, 21166, 22149, 22707, 23631, 23639, 25430, 25555, 32678, 33890, 34047, 35737, 38138, 38780, 40913, 40985, 40986, and 42623.

Two components present in a complex can be covalently linked, for example, present in a fusion protein, or cross-linked, e.g., treated with a crosslinking agent, or joined by a peptide or non-peptide linker as disclosed herein.

In some embodiments, two components present in a complex are part of the same fusion protein. Components can optionally be joined by a linker, such as a peptide linker or a non-peptide linker.

In some embodiments, a guide moiety or a part thereof (e.g., nuclease, such as dCas9) is joined to a heterologous gene effector by a linker. In some embodiments the guide moiety or part thereof is further joined to a second heterologous gene effector by a second linker that is the same or different. In some embodiments, a guide moiety or a part thereof (e.g., nuclease, such as dCas9) is fused to a heterologous gene effector without a linker.

In some embodiments, a guide moiety or a part thereof (e.g., nuclease, such as dCas9) is joined to an oligomerization domain or dimerization (e.g., heterodimerization) domain by a linker. In some embodiments the guide moiety or part thereof is further joined to a second oligomerization domain or dimerization (e.g., heterodimerization) domain by a second linker that is the same or different. In some embodiments, a guide moiety or a part thereof (e.g., nuclease, such as dCas9) is fused to a second oligomerization domain or dimerization (e.g., heterodimerization) domain without a linker.

In some embodiments, heterologous gene effector is joined to a second heterologous gene effector by a linker. In some embodiments the heterologous gene effector is further joined to a third heterologous gene effector by a second linker that is the same or different. In some embodiments, a heterologous gene effector is fused to a second heterologous gene effector without a linker.

In some embodiments, heterologous gene effector is joined to an oligomerization domain or dimerization (e.g., heterodimerization) domain by a linker. In some embodiments the heterologous gene effector is further joined to a second oligomerization domain or dimerization (e.g., heterodimerization) domain by a second linker that is the same or different. In some embodiments, a heterologous gene effector is fused to a second oligomerization domain or dimerization (e.g., heterodimerization) domain without a linker.

Any suitable linker can be used. A flexible linker can have a sequence containing stretches of glycine and serine residues. The small size of the glycine and serine residues provides flexibility and allows for mobility of the connected functional domains. The incorporation of serine or threonine can maintain the stability of the linker in aqueous solutions by forming hydrogen bonds with the water molecules, thereby reducing unfavorable interactions between the linker and protein moieties. Flexible linkers can also contain additional amino acids such as threonine and alanine to maintain flexibility, as well as polar amino acids such as lysine and glutamine to improve solubility. A rigid linker can have, for example, an alpha helix-structure. An alpha-helical rigid linker can act as a spacer between protein domains. Non-limiting examples of linkers include the sequences in TABLE 2, and repeats thereof, for example, 1, 2, 3, 4, 5, 6, 7, 8, 9, or 10 repeats. SEQ ID NOs: 1-6 provide flexible linkers or subunits thereof. SEQ ID NOs: 7-10 provide rigid linkers or subunits thereof.

TABLE 2

| SEQ ID NO: | Sequence |
|---|---|
| 1 | GGGGS |
| 2 | GGGS |
| 3 | GG |
| 4 | KESGSVSSEQLAQFRSLD |
| 5 | EGKSSGSGSESKST |
| 6 | GSAGSAAGSGEF |
| 7 | EAAAK |
| 8 | EAAAR |
| 9 | PAPAP |
| 10 | AEAAAKEAAAKA |

A linker sequence can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues in length.

In some embodiments, a linker is at least 1, at least 2, at least 3, at least 5, at least 7, at least 9, at least 11, at least 13, at least 15, or at least 20 amino acids. In some embodiments, a linker is at most 5, at most 7, at most 9, at most 11, at most 13, at most 15, at most 20, at most 25, at most 30, at most 40, or at most 50 amino acids.

In some embodiments, non-peptide linkers are used. A non-peptide linker can be, for example a chemical linker. Two parts of a complex of the disclosure can be connected by a chemical linker. Each chemical linker of the disclosure can be alkylene, alkenylene, alkynylene, heteroalkylene, cycloalkylene, heterocycloalkylene, arylene, or heteroarylene, any of which is optionally substituted. In some embodiments, a chemical linker of the disclosure can be an ester, ether, amide, thioether, or polyethylenegly col (PEG). In some embodiments, a linker can reverse the order of the amino acids sequence in a compound, for example, so that the amino acid sequences linked by the linked are head-to-head, rather than head-to-tail. Non-limiting examples of such linkers include diesters of dicarboxylic acids, such as oxalyl diester, malonyl diester, succinyl diester, glutaryl diester, adipyl diester, pimetyl diester, fumaryl diester, maleyl diester, phthalyl diester, isophthalyl diester, and terephthalyl diester. Non-limiting examples of such linkers include diamides of dicarboxylic acids, such as oxalyl diamide, malonyl diamide, succinyl diamide, glutaryl diamide, adipyl diamide, pimetyl diamide, fumaryl diamide, maleyl diamide, phthalyl diamide, isophthalyl diamide, and terephthalyl diamide. Non-limiting examples of such linkers include diamides of diamino linkers, such as ethylene diamine, 1,2-di(methylamino)ethane, 1,3-diaminopropane, 1,3-di(methylamino)propane, 1,4-di(methylamino)butane, 1,5-di(methylamino)pentane, 1,6-di(methylamino)hexane, and pipyrizine. Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

Two components present in a complex can be non-covalently coupled, for example, by ionic bonds, hydrogen bonds, interactions mediated by oligomerization or dimerization domains disclosed herein, etc.

In some embodiments, a guide moiety or a part thereof (e.g., nuclease, such as dCas9) is joined to a heterologous gene effector by non-covalent coupling. In some embodiments the guide moiety or part thereof is further joined to a second heterologous gene effector by non-covalent coupling. In some embodiments the guide moiety or part thereof is joined to a first heterologous gene effector covalently (e.g., as a fusion protein, optionally with a linker), and the guide moiety or part thereof is further joined to a second heterologous gene effector by non-covalent coupling.

In some embodiments, a guide moiety or a part thereof (e.g., nuclease, such as dCas9) is joined to an oligomerization domain or dimerization (e.g., heterodimerization) domain by non-covalent coupling. In some embodiments the guide moiety or part thereof is further joined to a second oligomerization domain or dimerization (e.g., heterodimerization) domain by non-covalent coupling. In some embodiments, a guide moiety or a part thereof (e.g., nuclease, such as dCas9) is fused to a first oligomerization domain or dimerization (e.g., heterodimerization) domain by covalent coupling (e.g., fused, optionally by a linker) and is joined to a second oligomerization domain or dimerization (e.g., heterodimerization) domain by non-covalent coupling.

In some embodiments, a first component of a guide moiety (e.g., a guide nucleic acid) is joined to a second component of the guide moiety (e.g., nuclease) non-covalently. In some embodiments, a first component of a guide moiety (e.g., a guide nucleic acid) is joined to a second component of the guide moiety (e.g., nuclease) covalently.

Any combination of covalent and non-covalent coupling can be used in a complex of the disclosure, for example, one or more heterologous gene effectors can be fused to a guide moiety non-covalently, and one or more oligomerization domains can be bound to a component of the complex (e.g., nuclease) covalently.

In some embodiments, a polypeptide providing increased or decreased stability is fused to or otherwise associated with a component of a complex of the disclosure, e.g., a guide moiety or a heterologous gene effector. The fused polypeptide can be located at the N-terminus, the C-terminus, or internally within the fusion protein.

In some embodiments, one or more components of a complex of the disclosure is fused to a domain that directs desirable sub-cellular localization, for example, a nuclear localization signal or a protein for targeting to the inner nuclear membrane, outer nuclear membrane, Cajal body, nuclear speckle, nuclear pore complex, PML body, nucleolus, P granule, GW body, stress granule, sponge body, endoplasmic reticulum, mitochondria, etc.

In some embodiments, a complex of the disclosure comprises a first protein linked to a first oligomerization (e.g., dimerization) domain, and a second protein linked to a second oligomerization (e.g., dimerization) domain. In some embodiments, an oligomerization domain or a dimerization domain can comprise a peptide interaction domain, for example, systems utilizing sgRNA2.0, SAM, SunTag, RAB, FLAG-biotin, or inducible oligomerization (e.g., dimerization) systems disclosed herein.

Complexes comprising combinations of heterologous gene effectors

Disclosed herein, in some embodiments, are complexes that comprise two or more heterologous gene effectors, and systems and methods for identifying the same.

In some embodiments, recruitment of two or more heterologous gene effectors to a locus of interest as disclosed herein can result in superior modulation of transcription compared to either heterologous gene effector alone, for example, more potent and/or persistent modulation of target gene (e.g., target endogenous gene) expression.

In some embodiments, recruitment of two or more heterologous gene effectors to a locus of interest as disclosed herein in a complex of the disclosure can result in superior modulation of transcription compared to recruitment of the combination of heterologous gene effectors separately, i.e., not present in a complex of the disclosure. The superior modulation of transcription can comprise, for example, more potent and/or persistent modulation of target gene (e.g., target endogenous gene) expression (e.g., activation or repression).

In some embodiments, assay systems of the disclosure, for example, high throughput screens, are used to identify combinations of heterologous gene effectors that are suitable for achieving a desired result, for example, increased expression of a target gene (e.g., target endogenous gene) or set of genes, reduced expression of a target gene or set of genes, increased expression above a certain threshold, decreased expression below a certain threshold, persistence of expression above or below a desired threshold for a desirable amount of time, etc.

A combination of heterologous gene effectors that are present in a complex of the disclosure and/or are recruited to a locus that regulates expression of a target gene (e.g., target endogenous gene) can be a combination of factors that do not interact in normal in vivo contexts (for example, due to cell type source, organism, tissue specific expression, localization to a given sub-cellular compartment or organelle, co-factor, structural, or complex requirements, etc.), but nonetheless mediate desirable epigenetic and/or transcriptional effects when orthogonally recruited to a target locus of interest, for example in a complex of the disclosure.

In some embodiments, a combination of heterologous gene effectors that are present in a complex of the disclosure are from different sources, for example, from any two or more of a human protein, a viral protein, a mammalian protein, a protein that primarily localizes to the nucleus, a chromatin regulator, a factor that facilitates heterochromatin formation, a factor that modulates histones through methylation, a factor that modulates histones through acetylation, a factor that modulates histones through phosphorylation, a factor that modulates histones through ADP-ribosylation, a factor that modulates histones through glycosylation, a factor that modulates histones through SUMOylation, a factor that modulates histones through ubiquitination, a factor that modulates histones by remodeling histone structure, e.g., via an ATP hydrolysis-dependent process, a histone acetyltransferase, a histone lysine methyltransferase, a component of a chromatin remodeling complex, a transcriptional regulator, a transcriptional activator, a transcriptional repressor domain, a transcription factor, a mesenchymal stem cell transcription factor, an embryonic stem cell transcription factor, an induced pluripotent stem cell (iPSC) transcription factor, an epithelial stem cell transcription factor, a cancer stem cell transcription factor, a cancer-related transcription factor, an immune cell transcription factor, a nuclear receptor, a nuclear hormone receptor, a validated human virus transcriptional regulator, a factor from a genome of a virus (e.g., from a virus family, subfamily, genus, species etc. disclosed herein), a factor from a genome of a virus that is capable of zoonotic transmission to humans, a factor from a shared human/bat virus, a factor from a viral genome from a metagenomic survey, a factor from a virus found in the human gut, a factor from a virus found in extreme environments, a factor from a virus or protein class with a high degree of documented transcriptional regulator modularity, or another source disclosed herein.

In some embodiments, a combination of heterologous gene effectors that are present in a complex of the disclosure are from the same or similar sources, for example, two or more heterologous gene effectors each of which contains a sequence from a human protein, a viral protein, a mammalian protein, a protein that primarily localizes to the nucleus, a chromatin regulator, a factor that facilitates heterochromatin formation, a factor that modulates histones through methylation, a factor that modulates histones through acetylation, a factor that modulates histones through phosphorylation, a factor that modulates histones through ADP-ribosylation, a factor that modulates histones through glycosylation, a factor that modulates histones through SUMOylation, a factor that modulates histones through ubiquitination, a factor that modulates histones by remodeling histone structure, e.g., via an ATP hydrolysis-dependent process, a histone acetyltransferase, a histone lysine methyltransferase, a component of a chromatin remodeling complex, a transcriptional regulator, a transcriptional activator, a transcriptional repressor domain, a transcription factor, a mesenchymal stem cell transcription factor, an embryonic stem cell transcription factor, an induced pluripotent stem cell (iPSC) transcription factor, an epithelial stem cell transcription factor, a cancer stem cell transcription factor, a cancer-related transcription factor, an immune cell transcription factor, a nuclear receptor, a nuclear hormone receptor, a validated human virus transcriptional regulator, a factor from a genome of a virus (e.g., from a virus family, subfamily, genus, species etc. disclosed herein), a factor from a genome of a virus that is capable of zoonotic transmission to humans, a factor from a shared human/bat virus, a factor from a viral genome from a metagenomic survey, a factor from a virus found in the human gut, a factor from a virus found in extreme environments, a factor from a virus or protein class with a high degree of documented transcriptional regulator modularity, or another source disclosed herein.

In some embodiments, a complex comprises two or more heterologous gene effectors that are from the same or similar sources and two or more heterologous gene effectors that are from different sources.

Two heterologous gene effectors that are present in a complex of the disclosure can be covalently linked to the complex, for example, present in a fusion protein, or treated with a crosslinking agent, etc. in any manner as disclosed elsewhere herein.

Two heterologous gene effectors that are present in a complex of the disclosure can be non-covalently associated to the complex, for example, by ionic bonds, hydrogen bonds, using an inducible and/or reversible system, etc. in any manner as disclosed elsewhere herein.

Two heterologous gene effectors that are present in a complex of the disclosure can be associated with the complex by using an inducible system. In some embodiments, the inducible system is reversible, e.g., upon withdrawal of the inducing agent, or upon treating with a dissociating agent.

Inducible systems for associating complex components can comprise fusing dimerization, oligomerization, or multimerization domains to the proteins to be associated. In some embodiments, the dimerization, oligomerization, or multimerization domains are fused to the N-terminus of the protein to be associated. In some embodiments, the dimerization, oligomerization, or multimerization domains are fused to the C-terminus of the protein to be associated. In some embodiments, the dimerization, oligomerization, or multimerization domains are fused to the N-terminus and the C-terminus of the protein to be associated (e.g., the same or different dimerization, oligomerization, or multimerization domains at the N-terminus and the C-terminus). In some embodiments, the dimerization, oligomerization, or multimerization domains are added in-frame within the amino acid sequence of a protein to be associated.

An inducible system for associating complex components can be a chemically-inducible system. Non-limiting examples of chemically-inducible systems include small molecule inducible systems, systems based on tetracycline or doxycycline, systems based on ponasterone A, abscisic acid (ABA)-inducible ABI-PYL1, gibberellin (GA)-inducible GID1-GAI, rapamycin-inducible FKBP-FRB, a TMP-Htag induced HaloTag/DHFR dimerization system, a dimerization system using an enzyme-catalyzed reaction, and systems utilizing a combination of the inducible components.

An inducible system for associating complex components can be a light-inducible system (e.g., an optogenetic system). Non-limiting examples of light-inducible systems include phytochrome-based red light-inducible PHYB-PIF, cryptochrome-based blue light-inducible CRY2 PHR-CIBN, light oxygen voltage-based blue-light-inducible FKF1-GI, pMAG, nMAG, BphS, and systems utilizing a combination of the inducible components.

In some embodiments, components of a complex of the disclosure are associated using inducible and reversible heterodimeric protein pairs from Arabidopsis thaliana (PYL1-ABI and GID1-GAI). Fusing heterodimerization domains from this system to each of two separately-expressed polypeptides allows for association of the polypeptides upon treatment with an inducing agent (the plant hormones ABA and GA).

For example, by fusing one protein from a heterodimeric pair to a guide moiety (e.g., dCas9) and the other to a heterologous gene effector, recruitment of the effector to the guide moiety can be achieved by addition of the appropriate plant hormone. Recruitment of a second heterologous gene effector can similarly be achieved by fusing one protein from a second heterodimeric pair to the guide moiety and the other to the second heterologous gene effector. In some embodiments, the same system an alternative inducible system is used to associate components in a complex that does not contain two or more different heterologous gene effectors, for example, to reversibly induce complex formation between one heterologous gene effector and a nuclease.

In some embodiments, components of an inducible system can be transiently or permanently expressed in cells of the disclosure. For example, cells can be transduced to transiently or stably express a guide moiety (e.g., dCas9) with fusions of heterodimerization domains from GAI and ABI.

In some embodiments, compositions and methods of the disclosure utilize candidate heterologous gene effectors with a high degree of documented transcriptional regulator modularity. In some embodiments, heterologous gene effectors with a high degree of documented transcriptional regulator modularity can be useful in combination with other gene effectors, for example, can be more likely to facilitate combinatorial or synergistic effects on gene transcription. This system allows, for example, orthogonal recruitment of candidate effector domains to test thousands of possible combinations in an unbiased manner. The inducibility and reversibility of the system allows the persistence of observed effects on transcriptional activation or repression to be evaluated. Aspects of the system (for example, that allow AND, OR, NAND, and NOR logic operators and a diametric regulator that activates gene expression with one inducer and represses with another) are described in Gao et al., “Complex transcriptional modulation with orthogonal and inducible dCas9 regulators.” Nature methods 13.12 (2016): 1043-1049.

In some embodiments, a complex comprises a combination of a chromatin regulator and a transcriptional regulator. In some embodiments, a complex comprises a combination of a first chromatin regulator and a second chromatin regulator. In some embodiments, a complex comprises a combination of a first transcriptional regulator and a second transcriptional regulator.

In some embodiments, a complex comprises a combination of at least one chromatin regulator and at least one transcriptional regulator. In some embodiments, a complex comprises a combination of a first chromatin regulator and at least a second chromatin regulator. In some embodiments, a complex comprises a combination of a first transcriptional regulator and at least a second transcriptional regulator.

In some embodiments, a complex comprises a combination of a chromatin regulator with two transcriptional regulators. In some embodiments, a complex comprises a combination of a transcriptional regulator with two chromatin regulators. In some embodiments, a complex comprises a combination of three chromatin regulators. In some embodiments, a complex comprises a combination of three transcriptional regulators.

In some embodiments, a complex comprises a combination of at least one chromatin regulator with at least two transcriptional regulators. In some embodiments, a complex comprises a combination of at least one transcriptional regulator with at least two chromatin regulators. In some embodiments, a complex comprises a combination of at least three chromatin regulators. In some embodiments, a complex comprises a combination of at least three transcriptional regulators.

A complex of the disclosure can comprise any suitable number of heterologous gene effectors, for example, at least one, at least two, at least three, at least four, or at least five. In some embodiments, a complex of the disclosure comprises at most two, at most three, at most four, or at most five heterologous gene effectors. In some embodiments, a complex of the disclosure comprises 1-5, 1-4, 1-3, 1-2, 2-5, 2-4, 2-3, 3-5, 3-4, or 4-5 heterologous gene effectors. In some embodiments, a complex of the disclosure comprises at most one, at most two, at most three, at most four, at most five, at most six, at most seven, at most eight, at most nine, or at most ten heterologous gene effectors. In some embodiments, a complex of the disclosure comprises one, two, three, four, five, six, seven, eight, nine, or ten heterologous gene effectors. In some embodiments, a complex of the disclosure comprises one heterologous gene effector. In some embodiments, a complex of the disclosure comprises two heterologous gene effectors. In some embodiments, a complex of the disclosure comprises three heterologous gene effectors.

In some embodiments two heterologous gene effectors are present in a complex of the disclosure and/or are recruited to a locus that regulates expression of a target gene (e.g., target endogenous gene). In some embodiments three heterologous gene effectors are present in a complex of the disclosure and/or are recruited to a locus that regulates expression of a target gene (e.g., target endogenous gene). In some embodiments four heterologous gene effectors are present in a complex of the disclosure and/or are recruited to a locus that regulates expression of a target gene (e.g., target endogenous gene). In some embodiments five heterologous gene effectors are present in a complex of the disclosure and/or are recruited to a locus that regulates expression of a target gene (e.g., target endogenous gene).

When two or more heterologous gene effectors are present in a complex of the disclosure and/or are recruited to a locus that regulates expression of a target gene (e.g., target endogenous gene), the heterologous gene effectors can be the same or different. In some embodiments, two heterologous gene effectors that are present in a complex of the disclosure and/or are recruited to a locus that regulates expression of a target gene are different to each other (e.g., from derived from different proteins of origin). In some embodiments, three heterologous gene effectors that are present in a complex of the disclosure and/or are recruited to a locus that regulates expression of a target gene are different to each other (e.g., from derived from different proteins of origin).

In some embodiments, a complex of the disclosure comprises at least one heterologous gene effector that is not or does not contain a sequence from P300, TET1, TET2, TET3, and/or HSF1.

In some embodiments, a complex of the disclosure comprises at least one heterologous gene effector that is not or does not contain a sequence from VP64, P65, Rta, VPR, AD2, CR3, ELKF1, GATA4, PRVIE, p53, SP1, MYOD, MEF2C, TAX, PPAR-gamma, MED1, MED7, MED17, MED26, MED29, TBP, GTF2H-2D, GTF2B, CBP, HSF1, MS2-p65-HSF1, MS2-TET1, NLS-dCas9-VP64, P300, p65, PRDM9, PUFa-GADD45A-TET1, R2, SunTag-scFv-sfGFP-TET1CD, TET1, TET2, TET3, VP120, VP16, VP16, VP16, VP48, VP64, VP64 or p65 +/−HSF1 or MyoD1, and/or VPR (Vp64+p65+Rta).

In some embodiments, a complex of the disclosure comprises at least one heterologous gene effector that is not or does not contain a sequence from KRAB, Mad mSIN3 interaction domain (SID), ERF repressor domain (ERD), cat3a, last 301 amino acids of Dnmt3a Isoform 1, dCas9-KRAB-MeCP2, DNMT3A, DNMT3A, DNMT3A, DNMT3A R887E-DNMT3L, DNMT3A-DNMT3L, DNMT3B, EZH2, HDAC, KRAB-DNMT3A, KRAB-DNMT3A-DNMT3L, KRAB-DNMT3L, LSD1, M.SssI, MQ1, MQ1 Q147E, SID4×, and/or SuntTag-DNMT3A.

### Target Genes

The disclosure provides compositions, methods, and systems for modulating expression of target genes (e.g., target endogenous genes). For example, disclosed herein are complexes that comprise a guide moiety and one or more heterologous gene effectors that can increase or decrease an activity or expression level of a target gene.

In some embodiments, a target gene or regulatory sequence thereof is endogenous to a subject, for example, present in the subject's genome. In some embodiments, a target gene or regulatory sequence thereof is not part of an engineered reporter system.

In some embodiments, a target gene is exogenous to a host subject, for example, a pathogen target gene or an exogenous gene expressed as a result of a therapeutic intervention, such as a gene therapy and/or cell therapy. In some embodiments, a target gene is an exogenous reporter gene, such as a reporter gene disclosed herein (e.g., a fluorescent protein). In some embodiments, a target gene is an exogenous synthetic gene.

In some embodiments, a complex of the disclosure can increase expression of a target gene (e.g., upon introducing the complex into a cell or population of cells). In some embodiments, an expression level is an RNA expression level can be measured by, for example, RNAseq, qPCR, microarray, gene array, FISH, etc. In some embodiments, an expression level is a protein expression level can be measured by, for example, Western Blot, ELISA, multiplex immunoassay, mass spectrometry, NMR, proteomics, flow cytometry, mass cytometry, etc.

In some embodiments, a complex of the disclosure can increase expression of a target gene (e.g., upon introducing the complex into a cell or population of cells) by at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold.

In some embodiments, a complex of the disclosure can increase expression of a target gene (e.g., upon introducing the complex into a cell or population of cells) at most 50%, at most 60%, at most 70%, at most 80%, at most 90%, at most 2-fold, at most 3 fold, at most 4 fold, at most 5 fold, at most 6 fold, at most 7 fold, at most 8 fold, at most 9 fold, at most 10 fold, at most 11 fold, at most 12 fold, at most 13 fold, at most 14, at most 15 fold, at most 20 fold, at most 30 fold, at most 40 fold, at most 50 fold, at most 60 fold, at most 70 fold, at most 80 fold, at most 90 fold, at most 100 fold, at most 150 fold, at most 200 fold, at most 250 fold, at most 300 fold, at most 350 fold, at most 400 fold, at most 500 fold, at most 600 fold, at most 700 fold, at most 800 fold, at most 900 fold, at most 1000 fold, at most 1500 fold, at most 2000 fold, at most 3000 fold, at most 5000 fold, or at most 10000 fold.

In some embodiments, a complex of the disclosure can increase expression of a target gene (e.g., upon introducing the complex into a cell or population of cells) about 10%, about 20%, about 30%, about 40%, about 50%, about 60%, about 70%, about 80%, about 90%, about 2-fold, about 3 fold, about 4 fold, about 5 fold, about 6 fold, about 7 fold, about 8 fold, about 9 fold, about 10 fold, about 11 fold, about 12 fold, about 13 fold, about 14, about 15 fold, about 20 fold, about 30 fold, about 40 fold, about 50 fold, about 60 fold, about 70 fold, about 80 fold, about 90 fold, about 100 fold, about 150 fold, about 200 fold, about 250 fold, about 300 fold, about 350 fold, about 400 fold, about 500 fold, about 600 fold, about 700 fold, about 800 fold, about 900 fold, about 1000 fold, about 1500 fold, about 2000 fold, about 3000 fold, about 5000 fold, or about 10000 fold.

In some embodiments, a complex of the disclosure can increase an expression level of a target gene (e.g., upon introducing the complex into a cell or population of cells) from below a limit of detection to a detectable level.

In some embodiments, a complex of the disclosure can reduce expression of a target gene (e.g., upon introducing the complex into a cell or population of cells) by at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold.

In some embodiments, a complex of the disclosure can reduce expression of a target gene (e.g., upon introducing the complex into a cell or population of cells) at most 50%, at most 60%, at most 70%, at most 80%, at most 90%, at most 2-fold, at most 3 fold, at most 4 fold, at most 5 fold, at most 6 fold, at most 7 fold, at most 8 fold, at most 9 fold, at most 10 fold, at most 11 fold, at most 12 fold, at most 13 fold, at most 14, at most 15 fold, at most 20 fold, at most 30 fold, at most 40 fold, at most 50 fold, at most 60 fold, at most 70 fold, at most 80 fold, at most 90 fold, at most 100 fold, at most 150 fold, at most 200 fold, at most 250 fold, at most 300 fold, at most 350 fold, at most 400 fold, at most 500 fold, at most 600 fold, at most 700 fold, at most 800 fold, at most 900 fold, at most 1000 fold, at most 1500 fold, at most 2000 fold, at most 3000 fold, at most 5000 fold, or at most 10000 fold.

In some embodiments, a complex of the disclosure can reduce expression of a target gene (e.g., upon introducing the complex into a cell or population of cells) about 10%, about 20%, about 30%, about 40%, about 50%, about 60%, about 70%, about 80%, about 90%, about 2-fold, about 3 fold, about 4 fold, about 5 fold, about 6 fold, about 7 fold, about 8 fold, about 9 fold, about 10 fold, about 11 fold, about 12 fold, about 13 fold, about 14, about 15 fold, about 20 fold, about 30 fold, about 40 fold, about 50 fold, about 60 fold, about 70 fold, about 80 fold, about 90 fold, about 100 fold, about 150 fold, about 200 fold, about 250 fold, about 300 fold, about 350 fold, about 400 fold, about 500 fold, about 600 fold, about 700 fold, about 800 fold, about 900 fold, about 1000 fold, about 1500 fold, about 2000 fold, about 3000 fold, about 5000 fold, or about 10000 fold.

In some embodiments, a complex of the disclosure can reduce an expression level of a target gene (e.g., upon introducing the complex into a cell or population of cells) from a detectable level to below a limit of detection.

In some embodiments, the degree in change of expression is relative to before introducing the complex into the cell or population of cells. In some embodiments, the degree in change of expression is relative to a corresponding control cell or population of cells that are not treated with the complex. In some embodiments, the degree in change of expression is relative to a corresponding control cell or population of cells that are treated with an alternative complex or gene expression regulator, for example, a complex comprising an alternative heterologous gene effector or combination thereof, or a different agent that modulates expression of the target gene. In some embodiments, the degree in change of expression is relative to a corresponding cell or population thereof that is treated with a control agent comprising a heterologous gene effector that is from P300, TET1, TET2, TET3, HSF1, VP64, P65, Rta, VPR, AD2, CR3, ELKF1, GATA4, PRVIE, p53, SP1, MYOD, MEF2C, TAX, PPAR-gamma, MED1, MED7, MED17, MED26, MED29, TBP, GTF2H-2D, GTF2B, CBP, HSF1, MS2-p65-HSF1, MS2-TET1, NLS-dCas9-VP64, P300, p65, PRDM9, PUFa-GADD45A-TET1, R2, SunTag-scFv-sfGFP-TET1CD, TET1, TET2, TET3, VP120, VP16, VP16, VP16, VP48, VP64, VP64 or p65+/−HSF1 or MyoD1, and/or VPR (Vp64+p65+Rta), KRAB, Mad mSIN3 interaction domain (SID), ERF repressor domain (ERD), cat3a, last 301 amino acids of Dnmt3a Isoform 1, dCas9-KRAB-MeCP2, DNMT3A, DNMT3A, DNMT3A, DNMT3A R887E-DNMT3L, DNMT3A-DNMT3L, DNMT3B, EZH2, HDAC, KRAB-DNMT3A, KRAB-DNMT3A-DNMT3L, KRAB-DNMT3L, LSD1, M.SssI, MQ1, MQ1 Q147E, SID4×, and/or SuntTag-DNMT3A.

In some embodiments, the degree in change of expression is relative to a corresponding cell or population thereof that is treated with a control agent comprising a heterologous gene effector that is from VPR. In some embodiments, the degree in change of expression is relative to a corresponding cell or population thereof that is treated with a control agent comprising a heterologous gene effector that is from KRAB. In some embodiments, the degree in change of expression is relative to a corresponding cell or population thereof that is treated with a control agent comprising a heterologous gene effector that is from VP64. In some embodiments, the degree in change of expression is relative to a corresponding cell or population thereof that is treated with a control agent comprising a heterologous gene effector that is from Rta. In some embodiments, the degree in change of expression is relative to a corresponding cell or population thereof that is treated with a control agent comprising a heterologous gene effector that is from p65. In some embodiments, the degree in change of expression is relative to a corresponding cell or population thereof that is treated with a control agent comprising a heterologous gene effector that is KAL.

In some embodiments, a complex of the disclosure can increase an activity level of a target gene (e.g., upon introducing the complex into a cell or population of cells). An activity level can be determined by a suitable functional assay for the target gene in question depending on the functional characteristics of the target gene. For example, an activity level of a target gene that is a mitogen could be determined by measuring cell proliferation; an activity level of a target gene that induces apoptosis could be measured by an annexin V assay or other suitable cell death assay; an activity level of an anti-inflammatory cytokine could be measured by an LPS-induced cytokine release assay.

In some embodiments, a complex of the disclosure can increase an activity level of a target gene (e.g., upon introducing the complex into a cell or population of cells) by at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold.

In some embodiments, a complex of the disclosure can increase an activity level of a target gene (e.g., upon introducing the complex into a cell or population of cells) at most 50%, at most 60%, at most 70%, at most 80%, at most 90%, at most 2-fold, at most 3 fold, at most 4 fold, at most 5 fold, at most 6 fold, at most 7 fold, at most 8 fold, at most 9 fold, at most 10 fold, at most 11 fold, at most 12 fold, at most 13 fold, at most 14, at most 15 fold, at most 20 fold, at most 30 fold, at most 40 fold, at most 50 fold, at most 60 fold, at most 70 fold, at most 80 fold, at most 90 fold, at most 100 fold, at most 150 fold, at most 200 fold, at most 250 fold, at most 300 fold, at most 350 fold, at most 400 fold, at most 500 fold, at most 600 fold, at most 700 fold, at most 800 fold, at most 900 fold, at most 1000 fold, at most 1500 fold, at most 2000 fold, at most 3000 fold, at most 5000 fold, or at most 10000 fold.

In some embodiments, a complex of the disclosure can increase an activity level of a target gene (e.g., upon introducing the complex into a cell or population of cells) about 10%, about 20%, about 30%, about 40%, about 50%, about 60%, about 70%, about 80%, about 90%, about 2-fold, about 3 fold, about 4 fold, about 5 fold, about 6 fold, about 7 fold, about 8 fold, about 9 fold, about 10 fold, about 11 fold, about 12 fold, about 13 fold, about 14, about 15 fold, about 20 fold, about 30 fold, about 40 fold, about 50 fold, about 60 fold, about 70 fold, about 80 fold, about 90 fold, about 100 fold, about 150 fold, about 200 fold, about 250 fold, about 300 fold, about 350 fold, about 400 fold, about 500 fold, about 600 fold, about 700 fold, about 800 fold, about 900 fold, about 1000 fold, about 1500 fold, about 2000 fold, about 3000 fold, about 5000 fold, or about 10000 fold.

In some embodiments, a complex of the disclosure can increase an activity level of a target gene (e.g., upon introducing the complex into a cell or population of cells) from below a limit of detection to a detectable level.

In some embodiments, a complex of the disclosure can reduce an activity level of a target gene (e.g., upon introducing the complex into a cell or population of cells) by at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, at least 1500 fold, at least 2000 fold, or at least 3000 fold.

In some embodiments, a complex of the disclosure can reduce an activity level of a target gene (e.g., upon introducing the complex into a cell or population of cells) at most 50%, at most 60%, at most 70%, at most 80%, at most 90%, at most 2-fold, at most 3 fold, at most 4 fold, at most 5 fold, at most 6 fold, at most 7 fold, at most 8 fold, at most 9 fold, at most 10 fold, at most 11 fold, at most 12 fold, at most 13 fold, at most 14, at most 15 fold, at most 20 fold, at most 30 fold, at most 40 fold, at most 50 fold, at most 60 fold, at most 70 fold, at most 80 fold, at most 90 fold, at most 100 fold, at most 150 fold, at most 200 fold, at most 250 fold, at most 300 fold, at most 350 fold, at most 400 fold, at most 500 fold, at most 600 fold, at most 700 fold, at most 800 fold, at most 900 fold, at most 1000 fold, at most 1500 fold, at most 2000 fold, at most 3000 fold, at most 5000 fold, or at most 10000 fold.

In some embodiments, a complex of the disclosure can reduce an activity level of a target gene (e.g., upon introducing the complex into a cell or population of cells) about 10%, about 20%, about 30%, about 40%, about 50%, about 60%, about 70%, about 80%, about 90%, about 2-fold, about 3 fold, about 4 fold, about 5 fold, about 6 fold, about 7 fold, about 8 fold, about 9 fold, about 10 fold, about 11 fold, about 12 fold, about 13 fold, about 14, about 15 fold, about 20 fold, about 30 fold, about 40 fold, about 50 fold, about 60 fold, about 70 fold, about 80 fold, about 90 fold, about 100 fold, about 150 fold, about 200 fold, about 250 fold, about 300 fold, about 350 fold, about 400 fold, about 500 fold, about 600 fold, about 700 fold, about 800 fold, about 900 fold, about 1000 fold, about 1500 fold, about 2000 fold, about 3000 fold, about 5000 fold, or about 10000 fold.

In some embodiments, a complex of the disclosure can reduce an activity level of a target gene (e.g., upon introducing the complex into a cell or population of cells) from a detectable level to below a limit of detection.

In some embodiments, the degree in change of an activity level is relative to before introducing the complex into the cell or population of cells. In some embodiments, the degree in change of an activity level is relative to a corresponding control cell or population of cells that are not treated with the complex. In some embodiments, the degree in change of an activity level is relative to a corresponding control cell or population of cells that are treated with an alternative complex or gene expression regulator, for example, a complex comprising an alternative heterologous gene effector or combination thereof, or a different agent that modulates an activity level of the target gene (e.g., target endogenous gene). In some embodiments, the degree in change of an activity level is relative to a corresponding cell or population thereof that is treated with a control agent comprising a heterologous gene effector that is from P300, TET1, TET2, TET3, HSF1, VP64, P65, Rta, VPR, AD2, CR3, ELKF1, GATA4, PRVIE, p53, SP1, MYOD, MEF2C, TAX, PPAR-gamma, MED1, MED7, MED17, MED26, MED29, TBP, GTF2H-2D, GTF2B, CBP, HSF1, MS2-p65-HSF1, MS2-TET1, NLS-dCas9-VP64, P300, p65, PRDM9, PUFa-GADD45A-TET1, R2, SunTag-scFv-sfGFP-TET1CD, TET1, TET2, TET3, VP120, VP16, VP16, VP16, VP48, VP64, VP64 or p65+/−HSF1 or MyoD1, and/or VPR (Vp64+p65+Rta), KRAB, Mad mSIN3 interaction domain (SID), ERF repressor domain (ERD), cat3a, last 301 amino acids of Dnmt3a Isoform 1, dCas9-KRAB-MeCP2, DNMT3A, DNMT3A, DNMT3A, DNMT3A R887E-DNMT3L, DNMT3A-DNMT3L, DNMT3B, EZH2, HDAC, KRAB-DNMT3A, KRAB-DNMT3A-DNMT3L, KRAB-DNMT3L, LSD1, M.SssI, MQ1, MQ1 Q147E, SID4×, and/or SuntTag-DNMT3A.

In some embodiments, the degree in change of activity level is relative to a corresponding cell or population thereof that is treated with a control agent comprising a heterologous gene effector that is from VPR. In some embodiments, the degree in change of activity level is relative to a corresponding cell or population thereof that is treated with a control agent comprising a heterologous gene effector that is from KRAB. In some embodiments, the degree in change of activity level is relative to a corresponding cell or population thereof that is treated with a control agent comprising a heterologous gene effector that is from VP64. In some embodiments, the degree in change of activity level is relative to a corresponding cell or population thereof that is treated with a control agent comprising a heterologous gene effector that is from Rta. In some embodiments, the degree in change of activity level is relative to a corresponding cell or population thereof that is treated with a control agent comprising a heterologous gene effector that is from p65. In some embodiments, the degree in change of activity level is relative to a corresponding cell or population thereof that is treated with a control agent comprising a heterologous gene effector that is KAL.

Complexes of the disclosure can, in some cases, elicit changes in expression and/or activity level of a target gene (e.g., target endogenous gene) that persists for longer than can be achieved with alternative compositions and methods. In some embodiments, persistent modulation of gene expression is advantageous as compared to transient modulation.

In some embodiments, a complex of the disclosure can increase expression and/or activity level of a target gene (e.g., target endogenous gene) to above a certain threshold for a period of time that is at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold longer than a control.

In some embodiments, a complex of the disclosure can reduce expression and/or activity level of a target gene (e.g., target endogenous gene) to below a certain threshold for a period of time that is at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold longer than a control.

In some embodiments, transient modulation of gene expression is advantageous as compared to persistent modulation, for example, where persistent over-expression or under-expression would lead to toxicity or off-target effects. Complexes of the disclosure can, in some cases, elicit changes in expression and/or activity level of a target gene (e.g., target endogenous gene) that persists for shorter periods of time than can be achieved with alternative compositions and methods.

In some embodiments, a complex of the disclosure can increase expression and/or activity level of a target gene (e.g., target endogenous gene) to above a certain threshold for at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold less time than a control.

In some embodiments, a complex of the disclosure can reduce expression and/or activity level of a target gene (e.g., target endogenous gene) to below a certain threshold for at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold less time than a control.

A control for an amount of time that expression and/or activity level is modulated can be, for example, a corresponding control cell or population of cells that are treated with an alternative complex or gene expression regulator, for example, a complex comprising an alternative heterologous gene effector or combination thereof, or a different agent that modulates an activity level of the target gene (e.g., target endogenous gene). In some embodiments, the persistence in change of an expression and/or activity level is relative to a corresponding cell or population thereof that is treated with a control agent comprising a heterologous gene effector that is from P300, TET1, TET2, TET3, HSF1, VP64, P65, Rta, VPR, AD2, CR3, ELKF1, GATA4, PRVIE, p53, SP1, MYOD, MEF2C, TAX, PPAR-gamma, MED1, MED7, MED17, MED26, MED29, TBP, GTF2H-2D, GTF2B, CBP, HSF1, MS2-p65-HSF1, MS2-TET1, NLS-dCas9-VP64, P300, p65, PRDM9, PUFa-GADD45A-TET1, R2, SunTag-scFv-sfGFP-TET1CD, TET1, TET2, TET3, VP120, VP16, VP16, VP16, VP48, VP64, VP64 or p65+/−HSF1 or MyoD1, and/or VPR (Vp64+p65+Rta), KRAB, Mad mSIN3 interaction domain (SID), ERF repressor domain (ERD), cat3a, last 301 amino acids of Dnmt3a Isoform 1, dCas9-KRAB-MeCP2, DNMT3A, DNMT3A, DNMT3A, DNMT3A R887E-DNMT3L, DNMT3A-DNMT3L, DNMT3B, EZH2, HDAC, KRAB-DNMT3A, KRAB-DNMT3A-DNMT3L, KRAB-DNMT3L, LSD1, M.SssI, MQ1, MQ1 Q147E, SID4×, and/or SuntTag-DNMT3A.

In some embodiments, the persistence in change of an expression and/or activity level is relative to a corresponding cell or population thereof that is treated with a control agent comprising a heterologous gene effector that is from VPR. In some embodiments, the persistence in change of an expression and/or activity level is relative to a corresponding cell or population thereof that is treated with a control agent comprising a heterologous gene effector that is from KRAB. In some embodiments, the persistence in change of an expression and/or activity level is relative to a corresponding cell or population thereof that is treated with a control agent comprising a heterologous gene effector that is from VP64. In some embodiments, the persistence in change of an expression and/or activity level is relative to a corresponding cell or population thereof that is treated with a control agent comprising a heterologous gene effector that is from Rta. In some embodiments, the persistence in change of an expression and/or activity level is relative to a corresponding cell or population thereof that is treated with a control agent comprising a heterologous gene effector that is from p65. In some embodiments, the persistence in change of an expression and/or activity level is relative to a corresponding cell or population thereof that is treated with a control agent comprising a heterologous gene effector that is KAL.

In some embodiments, a complex of the disclosure can increase expression and/or activity level of a target gene (e.g., target endogenous gene) to above a certain threshold for at least 1 hour, at least 2 hours, at least 3 hours, at least 4 hours, at least 5 hours, at least 6 hours, at least 7 hours, at least 8 hours, at least 9 hours, at least 10 hours, at least 12 hours, at least 14 hours, at least 18 hours, at least 20 hours, at least 1 day, at least 2 days, at least 3 days, at least 4 days, at least 5 days, at least 6 days, at least 7 days, at least 8 days, at least 9 days, at least 10 days, at least 14 days, at least 21 days, at least 28 days, at least 5 weeks, at least 6 weeks, at least 7 weeks, at least 8 weeks, at least 9 weeks, at least 10 weeks, at least 12 weeks, at least 14 weeks, at least 18 weeks, at least 20 weeks, or at least 26 weeks. The threshold can be, for example, a baseline level observed prior to treatment with the complex or in a corresponding population of cells not treated with the complex.

In some embodiments, a complex of the disclosure can reduce expression and/or activity level of a target gene (e.g., target endogenous gene) to below a certain threshold for at least 1 hour, at least 2 hours, at least 3 hours, at least 4 hours, at least 5 hours, at least 6 hours, at least 7 hours, at least 8 hours, at least 9 hours, at least 10 hours, at least 12 hours, at least 14 hours, at least 18 hours, at least 20 hours, at least 1 day, at least 2 days, at least 3 days, at least 4 days, at least 5 days, at least 6 days, at least 7 days, at least 8 days, at least 9 days, at least 10 days, at least 14 days, at least 21 days, at least 28 days, at least 5 weeks, at least 6 weeks, at least 7 weeks, at least 8 weeks, at least 9 weeks, at least 10 weeks, at least 12 weeks, at least 14 weeks, at least 18 weeks, at least 20 weeks, or at least 26 weeks. The threshold can be, for example, a baseline level observed prior to treatment with the complex or in a corresponding population of cells not treated with the complex.

In some embodiments, a complex of the disclosure can increase expression and/or activity level of a target gene (e.g., target endogenous gene) to above a certain threshold for at most 1 hour, at most 2 hours, at most 3 hours, at most 4 hours, at most 5 hours, at most 6 hours, at most 7 hours, at most 8 hours, at most 9 hours, at most 10 hours, at most 12 hours, at most 14 hours, at most 18 hours, at most 20 hours, at most 1 day, at most 2 days, at most 3 days, at most 4 days, at most 5 days, at most 6 days, at most 7 days, at most 8 days, at most 9 days, at most 10 days, at most 14 days, at most 21 days, at most 28 days, at most 5 weeks, at most 6 weeks, at most 7 weeks, at most 8 weeks, at most 9 weeks, at most 10 weeks, at most 12 weeks, at most 14 weeks, at most 18 weeks, at most 20 weeks, or at most 26 weeks. The threshold can be, for example, a baseline level observed prior to treatment with the complex or in a corresponding population of cells not treated with the complex.

In some embodiments, a complex of the disclosure can reduce expression and/or activity level of a target gene (e.g., target endogenous gene) to below a certain threshold for at most 1 hour, at most 2 hours, at most 3 hours, at most 4 hours, at most 5 hours, at most 6 hours, at most 7 hours, at most 8 hours, at most 9 hours, at most 10 hours, at most 12 hours, at most 14 hours, at most 18 hours, at most 20 hours, at most 1 day, at most 2 days, at most 3 days, at most 4 days, at most 5 days, at most 6 days, at most 7 days, at most 8 days, at most 9 days, at most 10 days, at most 14 days, at most 21 days, at most 28 days, at most 5 weeks, at most 6 weeks, at most 7 weeks, at most 8 weeks, at most 9 weeks, at most 10 weeks, at most 12 weeks, at most 14 weeks, at most 18 weeks, at most 20 weeks, or at most 26 weeks. The threshold can be, for example, a baseline level observed prior to treatment with the complex or in a corresponding population of cells not treated with the complex.

In some embodiments, a complex of the disclosure can increase expression and/or activity level of a target gene (e.g., target endogenous gene) to above a certain threshold for about 1 hour, about 2 hours, about 3 hours, about 4 hours, about 5 hours, about 6 hours, about 7 hours, about 8 hours, about 9 hours, about 10 hours, about 12 hours, about 14 hours, about 18 hours, about 20 hours, about 1 day, about 2 days, about 3 days, about 4 days, about 5 days, about 6 days, about 7 days, about 8 days, about 9 days, about 10 days, about 14 days, about 21 days, about 28 days, about 5 weeks, about 6 weeks, about 7 weeks, about 8 weeks, about 9 weeks, about 10 weeks, about 12 weeks, about 14 weeks, about 18 weeks, about 20 weeks, or about 26 weeks. The threshold can be, for example, a baseline level observed prior to treatment with the complex or in a corresponding population of cells not treated with the complex.

In some embodiments, a complex of the disclosure can reduce expression and/or activity level of a target gene (e.g., target endogenous gene) to below a certain threshold for about 1 hour, about 2 hours, about 3 hours, about 4 hours, about 5 hours, about 6 hours, about 7 hours, about 8 hours, about 9 hours, about 10 hours, about 12 hours, about 14 hours, about 18 hours, about 20 hours, about 1 day, about 2 days, about 3 days, about 4 days, about 5 days, about 6 days, about 7 days, about 8 days, about 9 days, about 10 days, about 14 days, about 21 days, about 28 days, about 5 weeks, about 6 weeks, about 7 weeks, about 8 weeks, about 9 weeks, about 10 weeks, about 12 weeks, about 14 weeks, about 18 weeks, about 20 weeks, or about 26 weeks. The threshold can be, for example, a baseline level observed prior to treatment with the complex or in a corresponding population of cells not treated with the complex.

The target gene (e.g., target endogenous gene) or regulatory sequence thereof can be any suitable gene or regulatory sequence thereof that is present in a cell, such as a stem cell, hematopoietic cell, an immune cell, or other cell type disclosed herein.

The target gene (e.g., target endogenous gene) can be a gene involved in immune cell regulation. In some embodiments, the target gene (e.g., target endogenous gene) is associated with cancer. The target gene associated with cancer can be a cell cycle gene, cell response gene, apoptosis gene, or phagocytosis gene.

In some embodiments, the term target gene (e.g., target endogenous gene) can also include target gene regulatory sequences, for example, promoters, enhancers, repressors, silencers, insulators, cis-regulatory elements, trans-regulatory elements, epigenetic modification (e.g., DNA methylation) sites, etc. that can influence an expression or activity level of the target gene, for example, upon binding of a complex, heterologous gene effector, and/or other factors to the regulatory sequence. Target gene regulatory sequences can be physically located outside of the transcriptional unit or open reading frame that encodes a product of the target gene.

A target gene regulatory sequence can be an endogenous regulatory sequence, for example, an endogenous promoter, endogenous enhancer, endogenous repressor, endogenous silencer, endogenous insulator, endogenous cis-regulatory element, endogenous trans-regulatory element, endogenous epigenetic modification (e.g., DNA methylation) site, etc., that is operatively coupled to the target gene (e.g., target endogenous gene). In some embodiments, a target gene regulatory sequence does not contain a component of an engineered or synthetic reporter system. In some embodiments, a target gene regulatory sequence does not function as part of an engineered reporter system. In some embodiments, a target gene regulatory sequence does not contain an exogenous, engineered, or synthetic regulatory element, for example, does not contain a response element that is modulated by tetracycline or analogs thereof. In some embodiments, a target gene regulatory sequence does not contain a response element that is modulated by an exogenous, engineered, or synthetic factor, for example, a does not contain a response element that is modulated by a transactivator or reverse transactivator. In some embodiments, a target gene regulatory sequence does not contain an element that is responsive to a transactivator or reverse transactivator that functions as part of an engineered inducible system, repressible system, and/or reporter system. In some embodiments, a target gene regulatory sequence is not a component of a Tet off or tTA-dependent system. In some embodiments, a target gene regulatory sequence is not a component of a Tet On or rtTA-dependent system.

In some embodiments, a target gene regulatory sequence does not contain a nucleotide sequence that is exogenous to the subject or host cell. In some embodiments, a target gene regulatory sequence does not contain an engineered or artificially generated or introduced nucleotide sequence.

In some embodiments, a complex or a heterologous gene effector identified by methods of the disclosure effects a desirable change that is specific to a particular target gene (e.g., target endogenous gene). In some embodiments, a complex or a heterologous gene effector identified by methods of the disclosure effects a desirable change that is applicable to two or more target genes (e.g., target endogenous genes). In some embodiments, a complex or a heterologous gene effector identified by methods of the disclosure effects a desirable change that is applicable to three or more target genes (e.g., target endogenous genes). In some embodiments, a complex or a heterologous gene effector identified by methods of the disclosure effects a desirable change that is applicable to a class of target genes (e.g., target endogenous genes), for example, genes with overlapping functional roles, that function in the same pathway, or are responsive to similar endogenous stimuli. In some embodiments, a complex or a heterologous gene effector identified by methods of the disclosure effects a desirable change that is broadly applicable to a wide variety of target genes (e.g., target endogenous genes), for example, elicits an expression level that is above or below a certain threshold for multiple target genes when present in a complex with a suitable guide moiety to direct binding to the target gene or a regulatory sequence thereof.

In some embodiments, a target gene (e.g., target endogenous gene) is a gene that is over-expressed or under-expressed in a disease or condition. In some embodiments, a target gene is a gene that is over-expressed or under-expressed in a heritable genetic disease.

In some embodiments, a target gene (e.g., target endogenous gene) is a gene that is over-expressed or under-expressed in an autoimmune disease. In some embodiments, a target gene is a gene that is over-expressed or under-expressed in Acute disseminated encephalomyelitis, Acute motor axonal neuropathy, Addison's disease, Adiposis dolorosa, Adult-onset Still's disease, Alopecia areata, Ankylosing Spondylitis, Anti-Glomerular Basement Membrane nephritis, Anti-neutrophil cytoplasmic antibody-associated vasculitis, Anti-N-Methyl-D-Aspartate Receptor Encephalitis, Antiphospholipid syndrome, Antisynthetase syndrome, Aplastic anemia, Autoimmune Angioedema, Autoimmune Encephalitis, Autoimmune enteropathy, Autoimmune hemolytic anemia, Autoimmune hepatitis, Autoimmune inner ear disease, Autoimmune lymphoproliferative syndrome, Autoimmune neutropenia, Autoimmune oophoritis, Autoimmune orchitis, Autoimmune pancreatitis, Autoimmune polyendocrine syndrome, Autoimmune polyendocrine syndrome type 2, Autoimmune polyendocrine syndrome type 3, Autoimmune progesterone dermatitis, Autoimmune retinopathy, Autoimmune thrombocytopenic purpura, Autoimmune thyroiditis, Autoimmune urticaria, Autoimmune uveitis, Balo concentric sclerosis, Behget's disease, Bickerstaffs encephalitis, Bullous pemphigoid, Celiac disease, Chronic fatigue syndrome, Chronic inflammatory demyelinating polyneuropathy, Churg-Strauss syndrome, Cicatricial pemphigoid, Cogan syndrome, Cold agglutinin disease, Complex regional pain syndrome, CREST syndrome, Crohn's disease, Dermatitis herpetiformis, Dermatomyositis, Diabetes mellitus type 1, Discoid lupus erythematosus, Endometriosis, Enthesitis, Enthesitis-related arthritis, Eosinophilic esophagitis, Eosinophilic fasciitis, Epidermolysis bullosa acquisita, Erythema nodosum, Essential mixed cryoglobulinemia, Evans syndrome, Felty syndrome, Fibromyalgia, Gastritis, Gestational pemphigoid, Giant cell arteritis, Goodpasture syndrome, Graves' disease, Graves ophthalmopathy, Guillain-Barre syndrome, Hashimoto's Encephalopathy, Hashimoto Thyroiditis, Henoch-Schonlein purpura, Hidradenitis suppurativa, Idiopathic dilated cardiomyopathy, Idiopathic inflammatory demyelinating diseases, IgA nephropathy, IgG4-related systemic disease, Inclusion body myositis, Inflamatory Bowel Disease (IBD), Intermediate uveitis, Interstitial cystitis, Juvenile Arthritis, Kawasaki's disease, Lambert-Eaton myasthenic syndrome, Leukocytoclastic vasculitis, Lichen planus, Lichen sclerosus, Ligneous conjunctivitis, Linear IgA disease, Lupus nephritis, Lupus vasculitis, Lyme disease, Meniere's disease, Microscopic colitis, Microscopic polyangiitis, Mixed connective tissue disease, Mooren's ulcer, Morphea, Mucha-Habermann disease, Multiple sclerosis, Myasthenia gravis, Myocarditis, Myositis, Neuromyelitis optica, Neuromyotonia, Opsoclonus myoclonus syndrome, Optic neuritis, Ord's thyroiditis, Palindromic rheumatism, Paraneoplastic cerebellar degeneration, Parry Romberg syndrome, Parsonage-Turner syndrome, Pediatric Autoimmune Neuropsychiatric Disorder Associated with Streptococcus, Pemphigus vulgaris, Pernicious anemia, Pityriasis lichenoides et varioliformis acuta, POEMS syndrome, Polyarteritis nodosa, Polymyalgia rheumatica, Polymyositis, Postmyocardial infarction syndrome, Postpericardiotomy syndrome, Primary biliary cirrhosis, Primary immunodeficiency, Primary sclerosing cholangitis, Progressive inflammatory neuropathy, Psoriasis, Psoriatic arthritis, Pure red cell aplasia, Pyoderma gangrenosum, Raynaud's phenomenon, Reactive arthritis, Relapsing polychondritis, Restless leg syndrome, Retroperitoneal fibrosis, Rheumatic fever, Rheumatoid arthritis, Rheumatoid vasculitis, Sarcoidosis, Schnitzler syndrome, Scleroderma, Sjogren's syndrome, Stiff person syndrome, Subacute bacterial endocarditis, Susac's syndrome, Sydenham chorea, Sympathetic ophthalmia, Systemic Lupus Erythematosus, Systemic scleroderma, Thrombocytopenia, Tolosa-Hunt syndrome, Transverse myelitis, Ulcerative colitis, Undifferentiated connective tissue disease, Urticaria, Urticarial vasculitis, Vasculitis, or Vitiligo.

In some embodiments, a target gene (e.g., target endogenous gene) is a gene that is over-expressed or under-expressed in a cancer, for example, acute leukemia, astrocytomas, biliary cancer (cholangiocarcinoma), bone cancer, breast cancer, brain stem glioma, bronchioloalveolar cell lung cancer, cancer of the adrenal gland, cancer of the anal region, cancer of the bladder, cancer of the endocrine system, cancer of the esophagus, cancer of the head or neck, cancer of the kidney, cancer of the parathyroid gland, cancer of the penis, cancer of the pleural/peritoneal membranes, cancer of the salivary gland, cancer of the small intestine, cancer of the thyroid gland, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, cervical cancer, chronic leukemia, colon cancer, colorectal cancer, cutaneous melanoma, ependymoma, epidermoid tumors, Ewings sarcoma, gastric cancer, glioblastoma, glioblastoma multiforme, glioma, hematologic malignancies, hepatocellular (liver) carcinoma, hepatoma, Hodgkin's Disease, intraocular melanoma, Kaposi sarcoma, lung cancer, lymphomas, medulloblastoma, melanoma, meningioma, mesothelioma, multiple myeloma, muscle cancer, neoplasms of the central nervous system (CNS), neuronal cancer, small cell lung cancer, non-small cell lung cancer, osteosarcoma, ovarian cancer, pancreatic cancer, pediatric malignancies, pituitary adenoma, prostate cancer, rectal cancer, renal cell carcinoma, sarcoma of soft tissue, schwanoma, skin cancer, spinal axis tumors, squamous cell carcinomas, stomach cancer, synovial sarcoma, testicular cancer, uterine cancer, or tumors and their metastases, including refractory versions of any of the above cancers, or a combination thereof.

In some embodiments, a target gene (e.g., target endogenous gene) is a differentiation-associated gene, for example, SSEA1, SSEA3/4, SSEA5, TRA1-60/81, TRAl-85, TRA2-54, GCTM-2, TG343, TG30, CD9, CD29, CD133/prominin, CD140a, CD56, CD73, CD90, CD105, OCT4, NANOG, SOX2, CD30, CD50, AHR, Aiolos/IKZF3, CDX4, CREB, DNMT3A, DNMT3B, EGR1, Fox03, GATA-1, GATA-2, GATA-3, Helios, HES-1, HHEX, HIF-1 alpha/HIF1A, HMGB1/HMG-1, HMGB3, Ikaros, c-Jun, LMO2, LMO4, c-Maf, MafB, MEF2C, MYB, c-Myc, NFATC2, NFIL3/E4BP4, Nrf2, p53, PITX2, PRDM16/MEL1, Prox1, PU.1/Spi-1, RUNX1/CBFA2, SALL4, SCL/Tall, Smad2, Smad2/3, Smad4, Smad7, Spi-B, STAT Activators, STAT Inhibitors, STAT3, STAT4, STAT5a, STAT6, TSC22, DUX4, DUX4/DUX4c, DUX4c, EBF-1, EBF-2, EBF-3, ETV5, FoxC2, FoxF1, GATA-4, GATA-6, HMGA2, c-Jun, MYF-5, Myocardin, MyoD, Myogenin, NFATC2, p53, Pax3, PDX-1/IPF1, PLZF, PRDM16/MEL1, RUNX2/CBFA1, Smad1, Smad3, Smad4, Smad5, Smad8, Smad9, Snail, SOX2, SOX9, SOX11, STAT Activators, STAT Inhibitors, STAT1, STAT3, TBX18, Twist-1, Twist-2, Brachyury, EOMES, FoxC2, FoxD3, FoxF1, FoxH1, Fox01/FKHR, GATA-2, GATA-3, GBX2, Goosecoid, HES-1, HNF-3 alpha/FoxAl, c-Jun, KLF2, KLF4, KLF5, c-Maf, Max, MEF2C, MIXL1, MTF2, c-Myc, Nanog, NFkB/IkB Activators, NFkB/IkB Inhibitors, NFkB1, NFkB2, Oct-3/4, Otx2, p53, Pax2, Pax6, PRDM14, Rex-1/ZFP42, SALL1, SALL4, Smadi, Smad2, Smad2/3, Smad3, Smad4, Smad5, Smad8, Snail, SOX2, SOX7, SOX15, SOX17, STAT Activators, STAT Inhibitors, STAT3, SUZ12, TBX6, TCF-3/E2A, THAP11, UTF1, WDR5, WT1, ZNF206, ZNF281, KLF2, KLF4, c-Maf, c-Myc, Nanog, Oct-3/4, p53, SOX1, SOX2, SOX3, SOX15, SOX18, TBX18, ASCL2/Mash2, CDX2, DNMT1, ELF3, Ets-1, FoxM1, FoxN1, GATA-6, Hairless, HNF-4 alpha/NR2A1, IRF6, c-Maf, MITF, Miz-1/ZBTB17, MSX1, MSX2, MYB, c-Myc, Neurogenin-3, NFATC1, NKX3.1, Nrf2, p53, p63/TP73L, Pax2, Pax3, RUNX1/CBFA2, RUNX2/CBFA1, RUNX3/CBFA3, Smadi, Smad2, Smad2/3, Smad4, Smad5, Smad7, Smad8, Snail, SOX2, SOX9, STAT Activators, STAT Inhibitors, STAT3, SUZ12, TCF-3/E2A, TCF7/TCF1, Androgen R/NR3C4, AP-2 gamma, beta-Catenin, beta-Catenin Inhibitors, Brachyury, CREB, ER alpha/NR3A1, ER beta/NR3A2, FoxM1, Fox03, FRA-1, GLI-1, GLI-2, GLI-3, HIF-1 alpha/HIF1A, HIF-2 alpha/EPAS1, HMGA1B, c-Jun, JunB, KLF4, c-Maf, MCM2, MCM7, MITF, c-Myc, Nanog, NFkB/IkB Activators, NFkB/IkB Inhibitors, NFkB1, NKX3.1, Oct-3/4, p53, PRDM14, Snail, SOX2, SOX9, STAT Activators, STAT Inhibitors, STAT3, TAZ/WWTR1, TBX3, Twist-1, Twist-2, WTi, or ZEBi.

In some embodiments, a heterologous gene effector is from a gene product that is a hematopoietic stem cell transcription factor. In some embodiments, a target gene is a mesenchymal stem cell transcription factor. In some embodiments, a target gene is an embryonic stem cell transcription factor. In some embodiments, a target gene is an induced pluripotent stem cell (iPSC) transcription factor. In some embodiments, a target gene is an epithelial stem cell transcription factor. In some embodiments, a target gene is a cancer stem cell transcription factor.

In some embodiments, a target gene is an age-related gene. In some embodiments, a target gene is a senescence-associated protein. In some embodiments, a target gene is a drug target.

In some embodiments, a target gene (e.g., target endogenous gene) is a cancer-related gene. Non-limiting examples of cancer-related genes include A1CF, ABIl, ABL1, ABL2, ACKR3, ACSL3, ACSL6, ACVR1, ACVR2A, AFDN, AFF1, AFF3, AFF4, AKAP9, AKT1, AKT2, AKT3, ALDH2, ALK, AMERI, ANK1, APC, APOBEC3B, AR, ARAF, ARHGAP26, ARHGAP5, ARHGEF10, ARHGEF10L, ARHGEF12, ARID1A, ARID1B, ARID2, ARNT, ASPSCR1, ASXL1, ASXL2, ATF1, ATIC, ATM, ATP1A1, ATP2B3, ATR, ATRX, AXIN1, AXIN2, B2M, BAP1, BARD1, BAX, BAZ1A, BCL10, BCL11A, BCL11B, BCL2, BCL2L12, BCL3, BCL6, BCL7A, BCL9, BCL9L, BCLAF1, BCOR, BCORL1, BCR, BIRC3, BIRC6, BLM, BMP5, BMPR1A, BRAF, BRCA1, BRCA2, BRD3, BRD4, BRIP1, BTG1, BTK, BUB1B, C15orf65, CACNAID, CALR, CAMTA1, CANT1, CARD11, CARS, CASP3, CASP8, CASP9, CBFA2T3, CBFB, CBL, CBLB, CBLC, CCDC6, CCNB1IP1, CCNC, CCND1, CCND2, CCND3, CCNE1, CCR4, CCR7, CD209, CD274, CD28, CD74, CD79A, CD79B, CDC73, CDH1, CDH10, CDH11, CDH17, CDK12, CDK4, CDK6, CDKN1A, CDKN1B, CDKN2A, CDKN2C, CDX2, CEBPA, CEP89, CHCHD7, CHD2, CHD4, CHEK2, CHIC2, CHST11, CIC, CIITA, CLIP1, CLP1, CLTC, CLTCL1, CNBD1, CNBP, CNOT3, CNTNAP2, CNTRL, COL1A1, COL2A1, COL3A1, COX6C, CPEB3, CREB1, CREB3L1, CREB3L2, CREBBP, CRLF2, CRNKL1, CRTC1, CRTC3, CSF1R, CSF3R, CSMD3, CTCF, CTNNA2, CTNNB1, CTNND1, CTNND2, CUL3, CUX1, CXCR4, CYLD, CYP2C8, CYSLTR2, DAXX, DCAF12L2, DCC, DCTN1, DDB2, DDIT3, DDR2, DDX10, DDX3X, DDX5, DDX6, DEK, DGCR8, DICERI, DNAJB1, DNM2, DNMT3A, DROSHA, DUX4L1, EBF1, ECT2L, EED, EGFR, EIFIAX, EIF3E, EIF4A2, ELF3, ELF4, ELK4, ELL, ELN, EML4, EP300, EPAS1, EPHA3, EPHA7, EPS15, ERBB2, ERBB3, ERBB4, ERC1, ERCC2, ERCC3, ERCC4, ERCC5, ERG, ESRI, ETNK1, ETV1, ETV4, ETV5, ETV6, EWSR1, EXT1, EXT2, EZH2, EZR, FAM131B, FAM135B, FAM47C, FANCA, FANCC, FANCD2, FANCE, FANCF, FANCG, FAS, FAT1, FAT3, FAT4, FBLN2, FBXO11, FBXW7, FCGR2B, FCRL4, FEN1, FES, FEV, FGFR1, FGFR10P, FGFR2, FGFR3, FGFR4, FH, FHIT, FIP1L1, FKBP9, FLCN, FLIl, FLNA, FLT3, FLT4, FNBP1, FOXA1, FOXL2, FOXO1, FOXO3, FOXO4, FOXP1, FOXR1, FSTL3, FUBP1, FUS, GAS7, GATA1, GATA2, GATA3, GLIl, GMPS, GNA11, GNAQ, GNAS, GOLGA5, GOPC, GPC3, GPC5, GPHN, GRIN2A, GRM3, H3F3A, H3F3B, HERPUD1, HEY1, HIF1A, HIP1, HIST1H3B, HIST1H4I, HLA-A, HLF, HMGA1, HMGA2, HMGN2P46, HNF1A, HNRNPA2B1, HOOK3, HOXA11, HOXA13, HOXA9, HOXC11, HOXC13, HOXD11, HOXD13, HRAS, HSP90AA1, HSP90AB1, ID3, IDH1, IDH2, IGF2BP2, IGH, IGK, IGL, IKBKB, IKZF1, IL2, IL21R, IL6ST, IL7R, IRF4, IRS4, ISX, ITGAV, ITK, JAK1, JAK2, JAK3, JAZF1, JUN, KAT6A, KAT6B, KAT7, KCNJ5, KDM5A, KDM5C, KDM6A, KDR, KDSR, KEAP1, KIAA1549, KIF5B, KIT, KLF4, KLF6, KLK2, KMT2A, KMT2C, KMT2D, KNL1, KNSTRN, KRAS, KTN1, LARP4B, LASP1, LATS1, LATS2, LCK, LCP1, LEF1, LEPROTLI, LHFPL6, LIFR, LMNA, LMO1, LMO2, LPP, LRIG3, LRP1B, LSM14A, LYL1, LZTR1, MACC1, MAF, MAFB, MALATI, MALT1, MAML2, MAP2K1, MAP2K2, MAP2K4, MAP3K1, MAP3K13, MAPK1, MAX, MB21D2, MDM2, MDM4, MDS2, MECOM, MED12, MEN1, MET, MGMT, MITF, MLF1, MLH1, MLLT1, MLLT10, MLLT11, MLLT3, MLLT6, MN1, MNX1, MPL, MRTFA, MSH2, MSH6, MS12, MSN, MTCP1, MTOR, MUC1, MUC16, MUC4, MUTYH, MYB, MYC, MYCL, MYCN, MYD88, MYH11, MYH9, MYO5A, MYOD1, N4BP2, NAB2, NACA, NBEA, NBN, NCKIPSD, NCOA1, NCOA2, NCOA4, NCOR1, NCOR2, NDRG1, NF1, NF2, NFATC2, NFE2L2, NFIB, NFKB2, NFKBIE, NIN, NKX2-1, NONO, NOTCHI, NOTCH2, NPM1, NR4A3, NRAS, NRG1, NSD1, NSD2, NSD3, NT5C2, NTHL1, NTRK1, NTRK3, NUMA1, NUP214, NUP98, NUTM1, NUTM2B, NUTM2D, OLIG2, OMD, P2RY8, PABPC1, PAFAH1B2, PALB2, PATZ1, PAX3, PAX5, PAX7, PAX8, PBRM1, PBX1, PCBP1, PCM1, PDCD1LG2, PDE4DIP, PDGFB, PDGFRA, PDGFRB, PERI, PHF6, PHOX2B, PICALM, PIK3CA, PIK3CB, PIK3R1, PIM1, PLAGI, PLCG1, PML, PMS1, PMS2, POLD1, POLE, POLG, POLQ, POT1, POU2AF1, POU5F1, PPARG, PPFIBP1, PPM1D, PPP2R1A, PPP6C, PRCC, PRDM1, PRDM16, PRDM2, PREX2, PRF1, PRKACA, PRKAR1A, PRKCB, PRPF40B, PRRX1, PSIP1, PTCH1, PTEN, PTK6, PTPN11, PTPN13, PTPN6, PTPRB, PTPRC, PTPRD, PTPRK, PTPRT, PWWP2A, QK1, RABEP1, RAC1, RAD17, RAD21, RAD51B, RAF1, RALGDS, RANBP2, RAP1GDS1, RARA, RB1, RBM10, RBM15, RECQL4, REL, RET, RFWD3, RGPD3, RGS7, RHOA, RHOH, RMI2, RNF213, RNF43, ROBO2, ROS1, RPL10, RPL22, RPL5, RPN1, RSPO2, RSPO3, RUNX1, RUNX1T1, S100A7, SALL4, SBDS, SDC4, SDHA, SDHAF2, SDHB, SDHC, SDHD, 44444, 44445, 44448, SET, SETBP1, SETD1B, SETD2, SETDB1, SF3B1, SFPQ, SFRP4, SGK1, SH2B3, SH3GL1, SHTN1, SIRPA, SIX1, SIX2, SK1, SLC34A2, SLC45A3, SMAD2, SMAD3, SMAD4, SMARCA4, SMARCB1, SMARCD1, SMARCEl, SMC1A, SMO, SND1, SNX29, SOCS1, SOX2, SOX21, SPECC1, SPEN, SPOP, SRC, SRGAP3, SRSF2, SRSF3, SS18, SS18L1, SSX1, SSX2, SSX4, STAG1, STAG2, STAT3, STAT5B, STAT6, STIL, STK11, STRN, SUFU, SUZ12, SYK, TAF15, TAL1, TAL2, TBL1XR1, TBX3, TCEA1, TCF12, TCF3, TCF7L2, TCL1A, TEC, TENT5C, TERT, TET1, TET2, TFE3, TFEB, TFG, TFPT, TFRC, TGFBR2, THRAP3, TLX1, TLX3, TMEM127, TMPRSS2, TNC, TNFAIP3, TNFRSF14, TNFRSF17, TOP1, TP53, TP63, TPM3, TPM4, TPR, TRA, TRAF7, TRB, TRD, TRIM24, TRIM27, TRIM33, TRIP11, TRRAP, TSC1, TSC2, TSHR, U2AF1, UBR5, USP44, USP6, USP8, VAV1, VHL, VTI1A, WAS, WDCP, WIF1, WNK2, WRN, WT1, WWTR1, XPA, XPC, XPO1, YWHAE, ZBTB16, ZCCHC8, ZEB1, ZFHX3, ZMYM2, ZMYM3, ZNF331, ZNF384, ZNF429, ZNF479, ZNF521, ZNRF3, and ZRSR2.

In some embodiments, a target gene (e.g., target endogenous gene) is an immune cell-related gene, for example, a cytokine, cytokine receptor, chemokine, chemokine receptor, co-inhibitory immune receptor, co-stimulatory immune receptor, immune cell transcription factor, etc.

In some embodiments, a target gene (e.g., target endogenous gene) is a cytokine, for example, 4-1BBL, APRIL, CD153, CD154, CD178, CD70, G-CSF, GITRL, GM-CSF, IFN-α, IFN-0, IFN-γ, IL-1RA, IL-la, IL-1, IL-2, IL-3, IL-4, IL-5, IL-6, IL-7, IL-9, IL-10, IL-11, IL-12, IL-13, IL-14, IL-15, IL-16, IL-17, IL-18, IL-20, IL-23, LIF, LIGHT, LT-0, M-CSF, MSP, OSM, OX40L, SCF, TALL-1, TGF-0, TGF-01, TGF-02, TGF-03, TNF-α, TNF-0, TRAIL, TRANCE, or TWEAK.

In some embodiments, a target gene (e.g., target endogenous gene) is a cytokine receptor, for example, A common gamma chain receptor, a common beta chain receptor, an interferon receptor, a TNF family receptor, a TGF-B receptor, Apo3, BCMA, CD114, CD115, CD116, CD117, CD118, CD120, CD120a, CD120b, CD121, CD121a, CD121b, CD122, CD123, CD124, CD126, CD127, CD130, CD131, CD132, CD212, CD213, CD213al, CD213a13, CD213a2, CD25, CD27, CD30, CD4, CD40, CD95 (Fas), CDwl19, CDwl21b, CDwl25, CDwl31, CDwl36, CDwl37 (41BB), CDw210, CDw217, GITR, HVEM, IL-11R, IL-11Ra, IL-14R, IL-15R, IL-15Ra, IL-18R, IL-18Ra, IL-18RO, IL-20R, IL-20Ra, IL-20RO, IL-9R, LIFR, LTOR, OPG, OSMR, OX40, RANK, TACI, TGF-OR1, TGF-OR2, TGF-OR3, TRAILRI, TRAILR2, TRAILR3, or TRAILR4.

In some embodiments, a target gene (e.g., target endogenous gene) is a chemokine, for example, ACT-2, AMAC-a, ATAC, ATAC, BLC, CCL1, CCL11, CCL13, CCL14, CCL15, CCL16, CCL17, CCL18, CCL19, CCL2, CCL20, CCL21, CCL22, CCL23, CCL24, CCL25, CCL26, CCL27, CCL3, CCL4, CCL5, CCL7, CCL8, CKb-6, CKb-8, CTACK, CX3CL1, CXCL1, CXCL10, CXCL11, CXCL12, CXCL13, CXCL14, CXCL2, CXCL3, CXCL4, CXCL5, CXCL6, CXCL7, CXCL8, CXCL9, DC-CK1, ELC, ENA-78, eotaxin, eotaxin-2, eotaxin-3, Eskine, exodus-1, exodus-2, exodus-3, fractalkine, GCP-2, GROa, GROb, GROg, HCC-i, HCC-2, HCC-4, 1-309, IL-8, ILC, IP-10, I-TAC, LAG-1, LARC, LCC-1, LD78u, LEC, Lkn-1, LMC, lymphoactin, lymphoactin b, MCAF, MCP-1, MCP-2, MCP-3, MCP-4, MDC, MDNCF, MGSA-a, MGSA-b, MGSA-g, Mig, MIP-1d, MIP-1a, MIP-10, MIP-2a, MIP-2b, MIP-3, MIP-3u, MIP-33, MIP-4, MIP-4a, MIP-5, MPIF-i, MPIF-2, NAF, NAP-1, NAP-2, oncostatin, PARC, PF4, PPBP, RANTES, SCM-la, SCM-lb, SDF-1α/β—, SLC, STCP-1, TARC, TECK, XCL1, or XCL2.

In some embodiments, a target gene (e.g., target endogenous gene) is a chemokine receptor, for example, CCRI, CCR2, CCR3, CCR4, CCR5, CCR6, CCR7, CCR8, CCR9, CCR10, CX3CR1, CXCR1, CXCR2, CXCR3, CXCR4, CXCR5, XCRI, or XCR1.

In some embodiments, a target gene (e.g., target endogenous gene) is an activating NK receptor, for example, CD100 (SEMA4D), CD16 (FcgRIIIA), CD160 (BY55), CD244 (2B4, SLAMF4), CD27, CD94—NKG2C, CD94—NKG2E, CD94-NKG2H, CD96, CRTAM, DAP12, DNAM1 (CD226), KIR2DL4, KIR2DS1, KIR2DS2, KIR2DS3, KIR2DS4, KIR2DS5, KIR3DS1, Ly49, NCR, NKG2D (KLRK1, CD314), NKp30 (NCR3), NKp44 (NCR2), NKp46 (NCR1), NKp80 (KLRF1, CLEC5C), NTB-A (SLAMF6), PSGL1, or SLAMF7 (CRACC, CS1, CD319).

In some embodiments, a target gene (e.g., target endogenous gene) is an inhibitory NK receptor, for example, CD161 (NKR-PiA, NK1.1), CD94-NKG2A, CD96, CEACAM1, KIR2DL1, KIR2DL2, KIR2DL3, KIR2DL4, KIR2DL5A, KIR2DL5B, KIR3DL1, KIR3DL2, KIR3DL3, KLRG1, LAIR1, LIR1 (ILT2, LILRB1), Ly49a, Ly49b, NKR-P1A (KLRB1), SIGLEC-10, SIGLEC-11, SIGLEC-14, SIGLEC-16, SIGLEC-3 (CD33), SIGLEC-5 (CD170), SIGLEC-6 (CD327), SIGLEC-7 (CD328), SIGLEC-8, SIGLEC-9 (CD329), SIGLEC-E, SIGLEC-F, SIGLEC-G, SIGLEC-H, or TIGIT.

In some embodiments, a target gene (e.g., target endogenous gene) is a co-inhibitory immune receptor, for example, 2B4, B7-1, BTLA, CD160, CTLA-4, DR6, Fas, LAG3, LAIR1, Ly108, PD-1, PD-L1, PD1H, TIGIT, TIM1, TIM2, or TIM3.

In some embodiments, a target gene (e.g., target endogenous gene) is co-stimulatory immune receptor, for example, 2B4, 4-1BB, CD2, CD4, CD8, CD21, CD27, CD28, CD30, CD40, CD84, CD226, CD355, CRACC, DcR3, DR3, GITR, HVEM, ICOS, Ly9, Ly108, LIGHT, LTOR, OX40, SLAM, TIM1, or TIM2.

In some embodiments, a target gene (e.g., target endogenous gene) is itself a gene effector, such as any of the gene effectors disclosed herein (e.g., a transcription factor disclosed herein).

In some embodiments, a target gene (e.g., target endogenous gene) is an immune cell transcription factor, for example, AP-1, Bcl6, E2A, EBF, Eomes, FoxP3, GATA3, Id2, Ikaros, IRF, IRF1, IRF2, IRF3, IRF3, IRF7, NFAT, NFkB, Pax5, PLZF, PU.1, ROR-gamma-T, STAT, STAT1, STAT2, STAT3, STAT4, STAT5, STAT5A, STAT5B, STAT6, T-bet, TCF7, or ThPOK.

In some embodiments, a target gene is a kinase, for example, a tyrosine kinase, or serine/threonine kinase. In some embodiments, a target gene is a phosphatase, for example, a tyrosine phosphatase, or serine/threonine phosphatase.

In some embodiments, a target gene is a receptor. In some embodiments, a target gene is an ion channel. In some embodiments, a target gene is a GPCR. In some embodiments, a target gene is a receptor tyrosine kinase. In some embodiments, a target gene is a ribosomal protein. In some embodiments, a target gene is a membrane protein. In some embodiments, a target gene is a cytoplasmic protein. In some embodiments, a target gene is a nuclear protein. In some embodiments, a target gene is a mitochondrial protein. In some embodiments, a target gene is a ubiquitin ligase. In some embodiments, a target gene is a methyltransferase. In some embodiments, a target gene is a glycosyltransferase. In some embodiments, a target gene is a hydrolase.

In some embodiments, CD45 is a target gene used in compositions and methods of the disclosure (e.g., for gene expression activation screens). In some embodiments, CD45 is not used as a target gene. Compositions and methods disclosed herein to identify complexes that modulate CD45 expression can similarly be modified and adapted to other target genes (e.g., target endogenous genes), including those disclosed herein.

In some embodiments, CD71 is a target gene used in compositions and methods of the disclosure (e.g., for gene expression reduction screens). In some embodiments, CD71 is not used as a target gene. Compositions and methods disclosed herein to identify complexes that modulate CD71 expression can similarly be modified and adapted to other target genes (e.g., target endogenous genes), including those disclosed herein.

### Libraries

Disclosed herein, in some aspects, are libraries of complexes. Libraries of the disclosure can be useful in screening assays to identify heterologous gene effectors, combinations thereof, and complexes containing the same that elicit desirable changes in expression and/or activity levels of target genes (e.g., target endogenous genes). Libraries of the disclosure can be assayed using methods disclosed herein in various cell types from various sources to identify heterologous gene effectors, combinations thereof, and complexes containing the same that are capable of eliciting the desirable changes in expression and/or activity levels of target gene(s) (e.g., target endogenous gene(s)) in the cell type of interest, for example, in cells from a certain subject, a particular tissue or lineage, a subject with a given disease or condition, etc.

Complexes that are members of a library can share a common attribute, such as sharing the same guide moiety, guide nucleic acid, source of heterologous gene effectors that are present in the library, class of target genes, etc. In some embodiments, where a library comprises complexes with two or more heterologous gene effectors per complex, one of the heterologous gene effectors can share a common attribute with other members of the library, while the other may not share a common attribute. In some embodiments, where a library comprises complexes with two or more heterologous gene effectors per complex, one of the heterologous gene effectors can share a first common attribute with other members of the library, and the second heterologous gene effector can share a second common attribute with other members of the library. In some embodiments, a library is designed without a common attribute amongst heterologous gene effectors, e.g., an unbiased library.

In some embodiments, a library of complexes comprises heterologous gene effectors from human sources. In some embodiments, a library of complexes comprises heterologous gene effectors from viral sources. In some embodiments, a library of complexes comprises heterologous gene effectors from other sources disclosed herein.

In some embodiments, a library of complexes comprises heterologous gene effectors from a particular source, for example, each of the heterologous gene effectors can be derived from a human protein, a viral protein, a mammalian protein, a protein that primarily localizes to the nucleus, a chromatin regulator, a factor that facilitates heterochromatin formation, a factor that modulates histones through methylation, a factor that modulates histones through acetylation, a factor that modulates histones through phosphorylation, a factor that modulates histones through ADP-ribosylation, a factor that modulates histones through glycosylation, a factor that modulates histones through SUMOylation, a factor that modulates histones through ubiquitination, a factor that modulates histones by remodeling histone structure, e.g., via an ATP hydrolysis-dependent process, a histone acetyltransferase, a histone lysine methyltransferase, a component of a chromatin remodeling complex, a transcriptional regulator, a transcriptional activator, a transcriptional repressor domain, a transcription factor, a mesenchymal stem cell transcription factor, an embryonic stem cell transcription factor, an induced pluripotent stem cell (iPSC) transcription factor, an epithelial stem cell transcription factor, a cancer stem cell transcription factor, a cancer-related transcription factor, an immune cell transcription factor, a nuclear receptor, a nuclear hormone receptor, a validated human virus transcriptional regulator, a factor from a genome of a virus (e.g., from a virus family, subfamily, genus, species etc. disclosed herein), a factor from a genome of a virus that is capable of zoonotic transmission to humans, a factor from a shared human/bat virus, a factor from a viral genome from a metagenomic survey, a factor from a virus found in the human gut, a factor from a virus found in extreme environments, a factor from a virus or protein class with a high degree of documented transcriptional regulator modularity, or another source disclosed herein.

In some embodiments, a library of complexes comprises heterologous gene effectors from a combination of sources from, e.g., any two or more of a human protein, a viral protein, a mammalian protein, a protein that primarily localizes to the nucleus, a chromatin regulator, a factor that facilitates heterochromatin formation, a factor that modulates histones through methylation, a factor that modulates histones through acetylation, a factor that modulates histones through phosphorylation, a factor that modulates histones through ADP-ribosylation, a factor that modulates histones through glycosylation, a factor that modulates histones through SUMOylation, a factor that modulates histones through ubiquitination, a factor that modulates histones by remodeling histone structure, e.g., via an ATP hydrolysis-dependent process, a histone acetyltransferase, a histone lysine methyltransferase, a component of a chromatin remodeling complex, a transcriptional regulator, a transcriptional activator, a transcriptional repressor domain, a transcription factor, a mesenchymal stem cell transcription factor, an embryonic stem cell transcription factor, an induced pluripotent stem cell (iPSC) transcription factor, an epithelial stem cell transcription factor, a cancer stem cell transcription factor, a cancer-related transcription factor, an immune cell transcription factor, a nuclear receptor, a nuclear hormone receptor, a validated human virus transcriptional regulator, a factor from a genome of a virus (e.g., from a virus family, subfamily, genus, species etc. disclosed herein), a factor from a genome of a virus that is capable of zoonotic transmission to humans, a factor from a shared human/bat virus, a factor from a viral genome from a metagenomic survey, a factor from a virus found in the human gut, a factor from a virus found in extreme environments, a factor from a virus or protein class with a high degree of documented transcriptional regulator modularity, or another source disclosed herein.

In some embodiments, a library of complexes comprises heterologous gene effectors from a particular subcellular localization, for example, factors that are capable of localizing or primarily localize to the nucleus of cells.

In some embodiments, an individual complex of a library comprises (i) a heterologous gene effector that is different from heterologous gene effectors in other complexes of the library; and (ii) a guide nucleic acid sequence that exhibits 100% sequence identity to guide nucleic acid sequences in the other complexes of the library.

In some embodiments, different individual complexes of a library comprise guide nucleic acids with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to guide nucleic acid sequences in the other complexes of the library. In some cases, even if the guide nucleic acid molecules (e.g., sgRNAs) in the library disclosed herein do not exhibit 100% sequence identity to each other (e.g., between about 80% and 99% sequence identity among each other), the guide nucleic acid molecules may be capable of binding and complexing with the same target gene (e.g., the same target polynucleotide sequence, such as the same genomic sequence).

In some embodiments, an individual complexes of a library comprises (i) a heterologous gene effector that is different to heterologous gene effectors present in other individual complexes in the library; (ii) a guide moiety (e.g., comprising a guide nucleic acid sequence) that is similar to or the same as other individual complexes in the library, for example, the same for all individual complexes in the library. In some embodiments, the guide moiety comprises a nuclease as disclosed herein, for example, an endonuclease that is heterologous with respect to the gene effector. The nuclease can be a nuclease-deficient or nuclease-dead nuclease of the disclosure. The nuclease can exhibit 100% sequence identity to heterologous nuclease of other complexes (e.g., all other complexes) in the library.

In some embodiments, an individual complexes of a library comprises (i) a first heterologous gene effector that is different to heterologous gene effectors present in other individual complexes in the library; (ii) a second heterologous gene effector that is the same as heterologous gene effectors present in other individual complexes in the library; and (iii) a guide moiety that is the same as other individual complexes in the library, for example, the same for all individual complexes in the library. The guide moiety can be or can comprise a guide nucleic acid that exhibits 100% identity to other (e.g., all) other individual complexes in the library. The guide moiety can comprise a nuclease (e.g., a heterologous endonuclease) that exhibits 100% sequence identity to heterologous nuclease of other complexes (e.g., all other complexes) in the library.

In some embodiments, an individual complexes of a library comprises (i) a first heterologous gene effector that is different to heterologous gene effectors present in other individual complexes in the library; (ii) a second heterologous gene effector that is the same as heterologous gene effectors present in other individual complexes in the library; (iii) a third heterologous gene effector that is the same as heterologous gene effectors present in other individual complexes in the library; and (iv) a guide moiety that is the same as other individual complexes in the library, for example, the same for all individual complexes in the library. The guide moiety can be or can comprise a guide nucleic acid that exhibits 100% identity to other (e.g., all other individual complexes) in the library. The guide moiety can comprise a nuclease (e.g., a heterologous endonuclease) that exhibits 100% sequence identity to heterologous nuclease of other complexes (e.g., all other complexes) in the library.

In some embodiments, an individual complexes of a library comprises (i) a first heterologous gene effector that is different to heterologous gene effectors present in other individual complexes in the library; (ii) a second heterologous gene effector that is different to heterologous gene effectors present in other individual complexes in the library; (iii) a third heterologous gene effector that is the same as heterologous gene effectors present in other individual complexes in the library; and (iv) a guide moiety that is the same as other individual complexes in the library, for example, the same for all individual complexes in the library. The guide moiety can be or can comprise a guide nucleic acid that exhibits 100% identity to other (e.g., all other individual complexes) in the library. The guide moiety can comprise a nuclease (e.g., a heterologous endonuclease) that exhibits 100% sequence identity to heterologous nuclease of other complexes (e.g., all other complexes) in the library.

In some embodiments, an individual complexes of a library comprises (i) a first heterologous gene effector that is different to heterologous gene effectors present in other individual complexes in the library; (ii) a second heterologous gene effector that is different to heterologous gene effectors present in other individual complexes in the library and the same as yet other individual complexes in the library; (iii) a third heterologous gene effector that is the same as heterologous gene effectors present in other individual complexes in the library; and (iv) a guide moiety that is the same as other individual complexes in the library, for example, the same for all individual complexes in the library. The guide moiety can be or can comprise a guide nucleic acid that exhibits 100% identity to other (e.g., all other) individual complexes in the library). The guide moiety can comprise a nuclease (e.g., a heterologous endonuclease) that exhibits 100% sequence identity to heterologous nuclease of other complexes (e.g., all other complexes) in the library.

In some embodiments, an individual complexes of a library comprises (i) a first heterologous gene effector that is different to heterologous gene effectors present in other individual complexes in the library; (ii) a second heterologous gene effector that is different to heterologous gene effectors present in other individual complexes in the library and the same as yet other individual complexes in the library; (iii) a third heterologous gene effector that is different to heterologous gene effectors present in other individual complexes in the library; and (iv) a guide moiety that is the same as other individual complexes in the library, for example, the same for all individual complexes in the library. The guide moiety can be or can comprise a guide nucleic acid that exhibits 100% identity to other (e.g., all other) individual complexes in the library). The guide moiety can comprise a nuclease (e.g., a heterologous endonuclease) that exhibits 100% sequence identity to heterologous nuclease of other complexes (e.g., all other complexes) in the library.

In some embodiments, an individual complexes of a library comprises (i) a heterologous gene effector that is different to heterologous gene effectors present in other individual complexes in the library; and (ii) a guide moiety (e.g., comprising a guide nucleic acid sequence) that is different to other individual complexes in the library. The guide moiety can comprise a guide nucleic acid sequence that is different to guide sequences present in other individual complexes in the library. The guide moiety can comprise a nuclease (e.g., a heterologous endonuclease) that exhibits 100% sequence identity to heterologous nuclease of other complexes (e.g., all other complexes) in the library. The guide moiety can comprise a nuclease (e.g., a heterologous endonuclease) that is different to other individual complexes in the library.

In some embodiments, individual complexes of a library (e.g., that comprise different heterologous gene effectors) specifically bind to the same target gene (e.g., target endogenous gene) or target gene regulatory sequence. In some embodiments, individual complexes of a library (e.g., that comprise the same and/or different heterologous gene effectors) specifically bind to different parts (e.g., subsequences) of a target gene or target gene regulatory sequence. In some embodiments, individual complexes of a library (e.g., that comprise the same and/or different heterologous gene effectors) specifically bind to different target gene regulatory sequences, each of which is capable of regulating expression of the same gene. In some embodiments, individual complexes of a library (e.g., that comprise the same and/or different heterologous gene effectors) specifically bind to different target genes (e.g., target endogenous genes) or target gene regulatory sequences.

A library of the disclosure can comprise, consist essentially of, or consist of any suitable number of different complexes for the intended purpose of the library. In some embodiments, “different complexes”, “individual complexes”, or “different individual complexes” can refer to members of the library that differ in composition from each other, for example, comprise different heterologous gene effectors (or combinations of heterologous gene effectors) compared to each other. In such cases multiple copies of the “different complexes”, “individual complexes”, or “different individual complexes” can be present in the library, and multiple copies of the same complex are not “different complexes”, “individual complexes”, or “different individual complexes”. For example, in a library can comprise 5 different complexes, each of which contains a different heterologous gene effector, and 100 copies of each complex can be present in the library, resulting in a library with 500 molecular complexes but only 5 “different complexes”.

In some embodiments, a library of the disclosure comprises at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, at least 2000, at least 2500, at least 3000, at least 3500, at least 4000, at least 4500, at least 5000, at least 5500, at least 6000, at least 6500, at least 7000, at least 7500, at least 8000, at least 8500, at least 9000, at least 9500, at least 10000, at least 10500, at least 11000, at least 11500, at least 12000, at least 12500, at least 13000, at least 13500, at least 14000, at least 14500, at least 15000, at least 15500, at least 16000, at least 16500, at least 17000, at least 17500, at least 18000, at least 18500, at least 19000, at least 19500, at least 20000, at least 20500, at least 21000, at least 21500, at least 22000, at least 22500, at least 23000, at least 23500, at least 24000, at least 24500, at least 25000, at least 25500, at least 26000, at least 26500, at least 27000, at least 27500, at least 28000, at least 28500, at least 29000, at least 29500, at least 30000, at least 35000, at least 40000, at least 45000, at least 50000, at least 60000, at least 70000, at least 80000, at least 90000, or at least 100000 different complexes.

In some embodiments, a library of the disclosure comprises at least 25 different complexes. In some embodiments, a library of the disclosure comprises at least 100 different complexes. In some embodiments, a library of the disclosure comprises at least 500 different complexes. In some embodiments, a library of the disclosure comprises at least 1000 different complexes. In some embodiments, a library of the disclosure comprises at least 15000 different complexes. In some embodiments, a library of the disclosure comprises at least 25000 different complexes.

In some embodiments, a library of the disclosure comprises at most 5, at most 10, at most 15, at most 20, at most 25, at most 30, at most 35, at most 40, at most 45, at most 50, at most 60, at most 70, at most 80, at most 90, at most 100, at most 110, at most 120, at most 130, at most 140, at most 150, at most 160, at most 170, at most 180, at most 190, at most 200, at most 250, at most 300, at most 350, at most 400, at most 450, at most 500, at most 550, at most 600, at most 650, at most 700, at most 750, at most 800, at most 850, at most 900, at most 950, at most 1000, at most 1100, at most 1200, at most 1300, at most 1400, at most 1500, at most 1600, at most 1700, at most 1800, at most 1900, at most 2000, at most 2500, at most 3000, at most 3500, at most 4000, at most 4500, at most 5000, at most 5500, at most 6000, at most 6500, at most 7000, at most 7500, at most 8000, at most 8500, at most 9000, at most 9500, at most 10000, at most 10500, at most 11000, at most 11500, at most 12000, at most 12500, at most 13000, at most 13500, at most 14000, at most 14500, at most 15000, at most 15500, at most 16000, at most 16500, at most 17000, at most 17500, at most 18000, at most 18500, at most 19000, at most 19500, at most 20000, at most 20500, at most 21000, at most 21500, at most 22000, at most 22500, at most 23000, at most 23500, at most 24000, at most 24500, at most 25000, at most 25500, at most 26000, at most 26500, at most 27000, at most 27500, at most 28000, at most 28500, at most 29000, at most 29500, at most 30000, at most 35000, at most 40000, at most 45000, at most 50000, at most 60000, at most 70000, at most 80000, at most 90000, or at most 100000 different complexes.

In some embodiments, a library of the disclosure comprises at most 100000 different complexes. In some embodiments, a library of the disclosure comprises at most 50000 different complexes. In some embodiments, a library of the disclosure comprises at most 30000 different complexes. In some embodiments, a library of the disclosure comprises at most 20000 different complexes.

In some embodiments, a library of the disclosure comprises at least 25 different complexes and at most 100, at most 110, at most 120, at most 130, at most 140, at most 150, at most 160, at most 170, at most 180, at most 190, at most 200, at most 250, at most 300, at most 350, at most 400, at most 450, at most 500, at most 550, at most 600, at most 650, at most 700, at most 750, at most 800, at most 850, at most 900, at most 950, at most 1000, at most 1100, at most 1200, at most 1300, at most 1400, at most 1500, at most 1600, at most 1700, at most 1800, at most 1900, at most 2000, at most 2500, at most 3000, at most 3500, at most 4000, at most 4500, at most 5000, at most 5500, at most 6000, at most 6500, at most 7000, at most 7500, at most 8000, at most 8500, at most 9000, at most 9500, at most 10000, at most 10500, at most 11000, at most 11500, at most 12000, at most 12500, at most 13000, at most 13500, at most 14000, at most 14500, at most 15000, at most 15500, at most 16000, at most 16500, at most 17000, at most 17500, at most 18000, at most 18500, at most 19000, at most 19500, at most 20000, at most 20500, at most 21000, at most 21500, at most 22000, at most 22500, at most 23000, at most 23500, at most 24000, at most 24500, at most 25000, at most 25500, at most 26000, at most 26500, at most 27000, at most 27500, at most 28000, at most 28500, at most 29000, at most 29500, at most 30000, at most 35000, at most 40000, at most 45000, at most 50000, at most 60000, at most 70000, at most 80000, at most 90000, or at most 100000 different complexes.

In some embodiments, a library of the disclosure comprises at least 100 different complexes and at most 100, at most 110, at most 120, at most 130, at most 140, at most 150, at most 160, at most 170, at most 180, at most 190, at most 200, at most 250, at most 300, at most 350, at most 400, at most 450, at most 500, at most 550, at most 600, at most 650, at most 700, at most 750, at most 800, at most 850, at most 900, at most 950, at most 1000, at most 1100, at most 1200, at most 1300, at most 1400, at most 1500, at most 1600, at most 1700, at most 1800, at most 1900, at most 2000, at most 2500, at most 3000, at most 3500, at most 4000, at most 4500, at most 5000, at most 5500, at most 6000, at most 6500, at most 7000, at most 7500, at most 8000, at most 8500, at most 9000, at most 9500, at most 10000, at most 10500, at most 11000, at most 11500, at most 12000, at most 12500, at most 13000, at most 13500, at most 14000, at most 14500, at most 15000, at most 15500, at most 16000, at most 16500, at most 17000, at most 17500, at most 18000, at most 18500, at most 19000, at most 19500, at most 20000, at most 20500, at most 21000, at most 21500, at most 22000, at most 22500, at most 23000, at most 23500, at most 24000, at most 24500, at most 25000, at most 25500, at most 26000, at most 26500, at most 27000, at most 27500, at most 28000, at most 28500, at most 29000, at most 29500, at most 30000, at most 35000, at most 40000, at most 45000, at most 50000, at most 60000, at most 70000, at most 80000, at most 90000, or at most 100000 different complexes.

In some embodiments, a library of the disclosure comprises at least 500 different complexes and at most 100, at most 110, at most 120, at most 130, at most 140, at most 150, at most 160, at most 170, at most 180, at most 190, at most 200, at most 250, at most 300, at most 350, at most 400, at most 450, at most 500, at most 550, at most 600, at most 650, at most 700, at most 750, at most 800, at most 850, at most 900, at most 950, at most 1000, at most 1100, at most 1200, at most 1300, at most 1400, at most 1500, at most 1600, at most 1700, at most 1800, at most 1900, at most 2000, at most 2500, at most 3000, at most 3500, at most 4000, at most 4500, at most 5000, at most 5500, at most 6000, at most 6500, at most 7000, at most 7500, at most 8000, at most 8500, at most 9000, at most 9500, at most 10000, at most 10500, at most 11000, at most 11500, at most 12000, at most 12500, at most 13000, at most 13500, at most 14000, at most 14500, at most 15000, at most 15500, at most 16000, at most 16500, at most 17000, at most 17500, at most 18000, at most 18500, at most 19000, at most 19500, at most 20000, at most 20500, at most 21000, at most 21500, at most 22000, at most 22500, at most 23000, at most 23500, at most 24000, at most 24500, at most 25000, at most 25500, at most 26000, at most 26500, at most 27000, at most 27500, at most 28000, at most 28500, at most 29000, at most 29500, at most 30000, at most 35000, at most 40000, at most 45000, at most 50000, at most 60000, at most 70000, at most 80000, at most 90000, or at most 100000 different complexes.

In some embodiments, a library of the disclosure comprises at least 1000 different complexes and at most 100, at most 110, at most 120, at most 130, at most 140, at most 150, at most 160, at most 170, at most 180, at most 190, at most 200, at most 250, at most 300, at most 350, at most 400, at most 450, at most 500, at most 550, at most 600, at most 650, at most 700, at most 750, at most 800, at most 850, at most 900, at most 950, at most 1000, at most 1100, at most 1200, at most 1300, at most 1400, at most 1500, at most 1600, at most 1700, at most 1800, at most 1900, at most 2000, at most 2500, at most 3000, at most 3500, at most 4000, at most 4500, at most 5000, at most 5500, at most 6000, at most 6500, at most 7000, at most 7500, at most 8000, at most 8500, at most 9000, at most 9500, at most 10000, at most 10500, at most 11000, at most 11500, at most 12000, at most 12500, at most 13000, at most 13500, at most 14000, at most 14500, at most 15000, at most 15500, at most 16000, at most 16500, at most 17000, at most 17500, at most 18000, at most 18500, at most 19000, at most 19500, at most 20000, at most 20500, at most 21000, at most 21500, at most 22000, at most 22500, at most 23000, at most 23500, at most 24000, at most 24500, at most 25000, at most 25500, at most 26000, at most 26500, at most 27000, at most 27500, at most 28000, at most 28500, at most 29000, at most 29500, at most 30000, at most 35000, at most 40000, at most 45000, at most 50000, at most 60000, at most 70000, at most 80000, at most 90000, or at most 100000 different complexes.

In some embodiments, a library of the disclosure comprises at least 5000 different complexes and at most 100, at most 110, at most 120, at most 130, at most 140, at most 150, at most 160, at most 170, at most 180, at most 190, at most 200, at most 250, at most 300, at most 350, at most 400, at most 450, at most 500, at most 550, at most 600, at most 650, at most 700, at most 750, at most 800, at most 850, at most 900, at most 950, at most 1000, at most 1100, at most 1200, at most 1300, at most 1400, at most 1500, at most 1600, at most 1700, at most 1800, at most 1900, at most 2000, at most 2500, at most 3000, at most 3500, at most 4000, at most 4500, at most 5000, at most 5500, at most 6000, at most 6500, at most 7000, at most 7500, at most 8000, at most 8500, at most 9000, at most 9500, at most 10000, at most 10500, at most 11000, at most 11500, at most 12000, at most 12500, at most 13000, at most 13500, at most 14000, at most 14500, at most 15000, at most 15500, at most 16000, at most 16500, at most 17000, at most 17500, at most 18000, at most 18500, at most 19000, at most 19500, at most 20000, at most 20500, at most 21000, at most 21500, at most 22000, at most 22500, at most 23000, at most 23500, at most 24000, at most 24500, at most 25000, at most 25500, at most 26000, at most 26500, at most 27000, at most 27500, at most 28000, at most 28500, at most 29000, at most 29500, at most 30000, at most 35000, at most 40000, at most 45000, at most 50000, at most 60000, at most 70000, at most 80000, at most 90000, or at most 100000 different complexes.

In some embodiments, a library of the disclosure comprises at least 10000 different complexes and at most 100, at most 110, at most 120, at most 130, at most 140, at most 150, at most 160, at most 170, at most 180, at most 190, at most 200, at most 250, at most 300, at most 350, at most 400, at most 450, at most 500, at most 550, at most 600, at most 650, at most 700, at most 750, at most 800, at most 850, at most 900, at most 950, at most 1000, at most 1100, at most 1200, at most 1300, at most 1400, at most 1500, at most 1600, at most 1700, at most 1800, at most 1900, at most 2000, at most 2500, at most 3000, at most 3500, at most 4000, at most 4500, at most 5000, at most 5500, at most 6000, at most 6500, at most 7000, at most 7500, at most 8000, at most 8500, at most 9000, at most 9500, at most 10000, at most 10500, at most 11000, at most 11500, at most 12000, at most 12500, at most 13000, at most 13500, at most 14000, at most 14500, at most 15000, at most 15500, at most 16000, at most 16500, at most 17000, at most 17500, at most 18000, at most 18500, at most 19000, at most 19500, at most 20000, at most 20500, at most 21000, at most 21500, at most 22000, at most 22500, at most 23000, at most 23500, at most 24000, at most 24500, at most 25000, at most 25500, at most 26000, at most 26500, at most 27000, at most 27500, at most 28000, at most 28500, at most 29000, at most 29500, at most 30000, at most 35000, at most 40000, at most 45000, at most 50000, at most 60000, at most 70000, at most 80000, at most 90000, or at most 100000 different complexes.

In some embodiments, a library of the disclosure comprises about 5, about 10, about 15, about 20, about 25, about 30, about 35, about 40, about 45, about 50, about 60, about 70, about 80, about 90, about 100, about 110, about 120, about 130, about 140, about 150, about 160, about 170, about 180, about 190, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2500, about 3000, about 3500, about 4000, about 4500, about 5000, about 5500, about 6000, about 6500, about 7000, about 7500, about 8000, about 8500, about 9000, about 9500, about 10000, about 10500, about 11000, about 11500, about 12000, about 12500, about 13000, about 13500, about 14000, about 14500, about 15000, about 15500, about 16000, about 16500, about 17000, about 17500, about 18000, about 18500, about 19000, about 19500, about 20000, about 20500, about 21000, about 21500, about 22000, about 22500, about 23000, about 23500, about 24000, about 24500, about 25000, about 25500, about 26000, about 26500, about 27000, about 27500, about 28000, about 28500, about 29000, about 29500, about 30000, about 35000, about 40000, about 45000, about 50000, about 60000, about 70000, about 80000, about 90000, or about 100000 different complexes.

In some embodiments, a library of the disclosure comprises about 10000 different complexes. In some embodiments, a library of the disclosure comprises about 16000 different complexes. In some embodiments, a library of the disclosure comprises about 28000 different complexes.

### Cells

Compositions, methods, and systems of the disclosure can be applied to cells of various types, and populations thereof. For example, a complex of the disclosure can be used to elicit changes in the expression or activity level of a target gene (e.g., target endogenous gene) in cells of a particular type, or populations thereof. Methods of the disclosure can be used to identify complexes that are capable of eliciting changes in the expression or activity of target genes (e.g., target endogenous genes) in cells of a particular type, or populations thereof.

In some embodiments, a complex or a heterologous gene effector identified by methods of the disclosure effects a desirable change in expression of a target gene (e.g., target endogenous gene) that is specific to a particular cell type. In some embodiments, a complex or a heterologous gene effector identified by methods of the disclosure effects a desirable change in expression of a target gene (e.g., target endogenous gene) that is applicable to two or more cell types. In some embodiments, a complex or a heterologous gene effector identified by methods of the disclosure effects a desirable change in expression of a target gene (e.g., target endogenous gene) that is applicable to three or more cell types. In some embodiments, a complex or a heterologous gene effector identified by methods of the disclosure effects a desirable change in expression of a target gene (e.g., target endogenous gene) that is applicable to a class of cell types, for example, cell types with overlapping functional roles, that are present in similar tissues, or that are from the same or similar differentiation lineages, e.g., stem cells, immune cells, T cells, T effector cells, etc. In some embodiments, a complex or a heterologous gene effector identified by methods of the disclosure effects a desirable change in expression of a target gene (e.g., target endogenous gene) that is broadly applicable to a wide variety of cell types, for example, elicits an expression level of a target gene that is above or below a certain threshold for multiple target cell types when introduced to the cells using suitable methods.

In some embodiments, a composition, complex, system, or method of the disclosure is used to effect a change in the expression or activity level of a target gene in a primary cell. In some embodiments, a composition, complex, system, or method of the disclosure is used to effect a change in the expression or activity level of a target gene in a cell line. In some embodiments, a composition, complex, system, or method of the disclosure is used to effect a change in the expression or activity level of a target gene in an immortalized cell.

In some embodiments, a composition, complex, system, or method of the disclosure is used to effect a change in the expression or activity level of a target gene (e.g., target endogenous gene) in a mammalian cell, for example, a human cell, non-human primate cell, non-rodent mammal cell, non-human mammal cell, swine cell, lagomorph cell, canine cell, etc. In some embodiments, a composition, complex, system, or method of the disclosure is used to effect a change in the expression or activity level of a target gene in a plant cell, an avian cell, a reptilian cell, a bacterial cell, or an archaeal cell.

A composition, complex, system, or method of the disclosure can be used to effect a change in the expression or activity level of a target gene (e.g., target endogenous gene) in a human cell.

A composition, complex, system, or method of the disclosure can be used to effect a change in the expression or activity level of a target gene (e.g., target endogenous gene) in a stem cell.

A composition, complex, system, or method of the disclosure can be used to effect a change in the expression or activity level of a target gene (e.g., target endogenous gene) in a differentiated cell.

A composition, complex, system, or method of the disclosure can be used to effect a change in the expression or activity level of a target gene (e.g., target endogenous gene) in a disease-associated cell.

A composition, complex, system, or method of the disclosure can be used to effect a change in the expression or activity level of a target gene (e.g., target endogenous gene) in a cancer cell.

A composition, complex, system, or method of the disclosure can be used to effect a change in the expression or activity level of a target gene (e.g., target endogenous gene) in a non-cancer cell.

A composition, complex, system, or method of the disclosure can be used to effect a change in the expression or activity level of a target gene (e.g., target endogenous gene) in a lymphoid cell, such as a B cell, a T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell, Reticulocyte, Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver cells (e.g., Hepatocyte, or Kupffer cell), Cartilage/bone/muscle; bone cells, including Osteoblast, Osteocyte, Osteoclast, teeth cells, (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte, Epidermal basal cell, Keratinocyte of fingernails and toenails, Nail bed basal cell, Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell, Wet stratified barrier epithelial cells, Surface epithelial cell of stratified squamous epithelium of cornea, tongue, oral cavity, esophagus, anal canal, distal urethra and vagina, basal cell of epithelia of cornea, tongue, oral cavity, esophagus, anal canal, distal urethra and vagina, Urinary epithelium cell, Exocrine secretory epithelial cells, Salivary gland mucous cell, Salivary gland serous cell, Von Ebner's gland cell in tongue, Mammary gland cell, Lacrimal gland cell, Ceruminous gland cell in ear, Eccrine sweat gland dark cell, Eccrine sweat gland clear cell. Apocrine sweat gland cell, Gland of Moll cell in eyelid, Sebaceous gland cell, Bowman's gland cell in nose, Brunner's gland cell in duodenum, Seminal vesicle cell, Prostate gland cell, Bulbourethral gland cell, Bartholin's gland cell, Gland of Littre cell, Uterus endometrium cell, Isolated goblet cell of respiratory and digestive tracts, Stomach lining mucous cell, Gastric gland zymogenic cell, Gastric gland oxyntic cell, Pancreatic acinar cell, Paneth cell of small intestine, Type II pneumocyte of lung, Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell, Macula densa cell of kidney, Metabolism and storage cells, Barrier function cells (e.g., Lung, Gut, Exocrine Glands and Urogenital Tract), Kidney, Type I pneumocyte, Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte, Megakaryocyte, Monocyte, Connective tissue macrophage (various types), Epidermal Langerhans cell, Osteoclast, Dendritic cell, Microglial cell, Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, neurons, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell, Spermatozoon, Nurse cells, Ovarian follicle cell, Sertoli cell, Thymus epithelial cell, Interstitial cells, Interstitial kidney cells, common myeloid progenitors, common lymphoid progenitors, and stem cells that are differentiated into or are to be differentiated into any cell type disclosed herein.

A composition, complex, system, or method of the disclosure can be used to effect a change in the expression or activity level of a target gene (e.g., target endogenous gene) in a stem cell, for example, an isolated stem cell (e.g., an ESC) or an induced stem cell (e.g., an iPSC).

A composition, complex, system, or method of the disclosure can be used to effect a change in the expression or activity level of a target gene (e.g., target endogenous gene) in a hematopoietic stem cell, for example, a hematopoietic stem cell from a subject, for example, from bone marrow, or peripheral blood (e.g., a mobilized peripheral blood apheresis product, for example, mobilized by administration of GCSF, GM-CSF, mozobil, or a combination thereof).

In some cases, pluripotency of stem cells (e.g., ESCs or iPSCs) can be determined, in part, by assessing pluripotency characteristics of the cells. Pluripotency characteristics can include, but are not limited to: pluripotent stem cell morphology; the potential for unlimited self-renewal; expression of pluripotent stem cell markers including, but not limited to SSEA1, SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; ability to differentiate to all three somatic lineages (ectoderm, mesoderm and endoderm); ability to form teratomas comprising the three somatic lineages; and/or (vi) formation of embryoid bodies comprising cells from the three somatic lineages.

A composition, complex, system, or method of the disclosure can be used to effect a change in the expression or activity level of a target gene (e.g., target endogenous gene) in an immune cell, for example, lymphocytes, T cells, CD4+T cells, CD8+T cells, alpha-beta T cells, gamma-delta T cells, T regulatory cells (Tregs), cytotoxic T lymphocytes, Th1 cells, Th2 cells, Th17 cells, Th9 cells, naive T cells, memory T cells, effector T cells, effector-memory T cells (TEM), central memory T cells (TCM), resident memory T cells (TRM), follicular helper T cells (TFH), Natural killer T cells (NKTs), tumor-infiltrating lymphocytes (TILs), Natural killer cells (NKs), Innate Lymphoid Cells (ILCs), ILC1 cells, ILC2 cells, ILC3 cells, lymphoid tissue inducer (LTi) cells, B cells, B1 cells, Bla cells, B1b cells, B2 cells, plasma cells, B regulatory cells, memory B cells, marginal zone B cells, follicular B cells, germinal center B cells, antigen presenting cells (APCs), monocytes, macrophages, M1 macrophages, M2 macrophages, tissue-associated macrophages, dendritic cells, plasmacytoid dendritic cells, neutrophils, mast cells, basophils, eosinophils, common myeloid progenitors, common lymphoid progenitors, or any combination thereof.

A composition, complex, system, or method of the disclosure can be used to effect a change in the expression or activity level of an engineered cell that is used to manufacture a biologic, for example, an antibody or other protein-based therapeutic.

### Assay Systems, Subjects, and Illustrative Applications

Assay systems of the disclosure allow for a systematic and large scale survey of heterologous gene effectors, combinations thereof, complexes comprising the heterologous gene effector(s), and libraries of the same, for example, to identify one or more lead heterologous gene effectors or complexes that elicits a desirable change in an expression or activity level of a target gene (e.g., target endogenous gene).

In some embodiments, compositions and methods of the disclosure can be used to identify one or more lead heterologous gene effectors of a library that effect a desirable change, for example, increased expression of a target gene (e.g., target endogenous gene) above a certain threshold, or decreased expression of a target gene to below a certain threshold.

In some embodiments, a heterologous gene effector identified by methods of the disclosure (e.g., any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052, or another sequence) is modified to comprise one or more amino acid insertions, deletions, or substitutions, and the impact of the mutation(s) on the change in expression elicited in the target gene is evaluated. Such a strategy can be used, for example, to refine the activity of the heterologous gene effector or to identify a heterologous gene effector sequence with improved properties compared to the initially identified sequence. In some embodiments, methods of the disclosure comprise identifying one or more lead heterologous gene effectors, mutating one or more amino acid residues of the heterologous gene effector (e.g., with one or more deletions, insertions, and/or substitutions, to arrive at a degree of sequence identity or sequence similarity to the original sequence as disclosed herein), and testing the impact of the one or more amino acid mutations on the change in expression of the target gene elicited by the heterologous gene effector. In some embodiments, the disclosure provides a heterologous gene effector that comprises one or more amino acid insertions, deletions, or substitutions relative to any one of SEQ ID NOs: 16-16154, any one of SEQ ID NOs: 16-13605, any one of SEQ ID NOs: 16155-47350, any one of SEQ ID NOs: 16155-43953, any one of SEQ ID NOs: 47351-49333, any one of SEQ ID NOs: 49353-50052, or any two or more of SEQ ID NOs: 16-49333 and 49353-50052.

In some embodiments, compositions and methods of the disclosure can be used to identify a combination of two or more heterologous gene effectors (e.g., that are present in the same complex) that effect a desirable change, for example, increased expression of a target gene (e.g., target endogenous gene) above a certain threshold, or decreased expression of a target gene to below a certain threshold.

In some embodiments, compositions and methods of the disclosure can be used to identify a combination of three or more heterologous gene effectors (e.g., that are present in the same complex) that effect a desirable change, for example, increased expression of a target gene (e.g., target endogenous gene) above a certain threshold, or decreased expression of a target gene (e.g., target endogenous gene) to below a certain threshold.

In some embodiments, an assay system is unbiased by design. In some embodiments, an assay system is targeted by design.

Complexes comprising the heterologous gene effector(s) can be delivered to cells using any suitable method.

In some embodiments, complexes comprising the heterologous gene effector(s) are delivered as nucleic acids that encode one or more components of the complex using any suitable method, for example, electroporation or use of suitable vectors such as viral vectors, liposomal vector, microparticles, nanoparticles, dendrimers, etc. In some embodiments, one or more components of a complex are delivered in a manner that results in transient expression, for example, transient transfection. In some embodiments, one or more components of a complex are delivered in a manner that results in persistent expression, for example, lentiviral transduction, genomic integration using nuclease systems disclosed herein, etc. Components of complexes can be delivered using separate vectors/methods or the same vector. In some embodiments, one or more components are delivered in a manner that results in persistent expression, for example, using lentiviral transduction, and one or more other components are delivered in a manner that results in transient expression.

In some embodiments, complexes comprising the heterologous gene effector(s) are delivered as proteins or ribonucleoproteins, for example, using a suitable vector, such as a liposomal vector, nanoparticle, viral vector, non-viral vector, etc.

Optionally, cells that express adequate levels of one or more components of a system or complex of the disclosure (e.g., guide moiety, guide nucleic acid, nuclease, heterologous gene effector, components of an inducible system, etc) can be enriched, e.g., by use of selectable markers (e.g., resistance or susceptibility genes) or by cell sorting, for example, based on expression of the component or based on expression of a reporter gene that is co-expressed with the component.

Expression or activity level of a target gene (e.g., target endogenous gene) can be measured any suitable amount of time after delivery of a complex or component of a complex to cells. In some embodiments, expression or activity level of a target gene is measured at least 1 hour, at least 2 hours, at least 3 hours, at least 4 hours, at least 5 hours, at least 6 hours, at least 7 hours, at least 8 hours, at least 9 hours, at least 10 hours, at least 12 hours, at least 14 hours, at least 18 hours, at least 20 hours, at least 1 day, at least 2 days, at least 3 days, at least 4 days, at least 5 days, at least 6 days, at least 7 days, at least 8 days, at least 9 days, at least 10 days, at least 14 days, at least 21 days, at least 28 days, at least 5 weeks, at least 6 weeks, at least 7 weeks, at least 8 weeks, at least 9 weeks, at least 10 weeks, at least 12 weeks, at least 14 weeks, at least 18 weeks, at least 20 weeks, or at least 26 weeks after delivery of the complex or component of a complex to the cells.

In some embodiments, expression or activity level of a target gene (e.g., target endogenous gene) is measured at most 1 hour, at most 2 hours, at most 3 hours, at most 4 hours, at most 5 hours, at most 6 hours, at most 7 hours, at most 8 hours, at most 9 hours, at most 10 hours, at most 12 hours, at most 14 hours, at most 18 hours, at most 20 hours, at most 1 day, at most 2 days, at most 3 days, at most 4 days, at most 5 days, at most 6 days, at most 7 days, at most 8 days, at most 9 days, at most 10 days, at most 14 days, at most 21 days, at most 28 days, at most 5 weeks, at most 6 weeks, at most 7 weeks, at most 8 weeks, at most 9 weeks, at most 10 weeks, at most 12 weeks, at most 14 weeks, at most 18 weeks, at most 20 weeks, or at most 26 weeks after delivery of the complex or component of a complex to the cells.

In some embodiments, expression or activity level of a target gene (e.g., target endogenous gene) is measured about 1 hour, about 2 hours, about 3 hours, about 4 hours, about 5 hours, about 6 hours, about 7 hours, about 8 hours, about 9 hours, about 10 hours, about 12 hours, about 14 hours, about 18 hours, about 20 hours, about 1 day, about 2 days, about 3 days, about 4 days, about 5 days, about 6 days, about 7 days, about 8 days, about 9 days, about 10 days, about 14 days, about 21 days, about 28 days, about 5 weeks, about 6 weeks, about 7 weeks, about 8 weeks, about 9 weeks, about 10 weeks, about 12 weeks, about 14 weeks, about 18 weeks, about 20 weeks, or about 26 weeks after delivery of the complex or component of a complex to the cells.

In some embodiments, an expression level is a protein expression level can be measured by, for example, Western Blot, ELISA, multiplex immunoassay, mass spectrometry, NMR, proteomics, flow cytometry, mass cytometry, etc.

In some embodiments, a complex of the disclosure can increase expression of a target gene (e.g., upon introducing the complex into a cell or population of cells). In some embodiments, an expression level is an RNA expression level can be measured by, for example, RNAseq, qPCR, microarray, gene array, FISH, etc.

Cells that express high or low levels of a target gene (e.g., target endogenous gene) can optionally be enriched by sorting, for example, using fluorescent activated cell sorting, magnetic activated cell sorting, or a combination thereof. Cells having high or low activity levels of a target gene can be identified using functional assays.

One or more lead heterologous gene effectors or complexes that elicits a desirable change in an expression or activity level of a target gene (e.g., target endogenous gene) can be identified based on the use of unique molecular identifiers (e.g., barcodes) that are designed to correspond to specific heterologous gene effectors, combinations thereof, complexes, etc.

Unique molecular identifiers can be short sequences used to uniquely tag distinct constructs, e.g., each heterologous gene effector can be associated with a unique molecular identifier in a pre-determined manner, and the information can be stored to allow mapping of an unique molecular identifier to a specific heterologous gene effector, e.g., when the unique molecular identifier is found by sequencing. The unique molecular identifier can be synthesized together with the coding sequence for the heterologous gene effector as disclosed herein, e.g., for cloning into a vector, such as a lentiviral vector, an expression plasmid, a sequence to be integrated into the genome, etc. A unique molecular identifier can be any suitable length, for example, about 8-30, about 8-25, about 8-20, about 8-15, about 10-30, about 10-25, about 10-20, about 10-15 nucleotides in length. A unique molecular identifier can be about 12 nucleotides in length.

In some embodiments, a single unique molecular identifier is used to tag and identify a heterologous gene effector. In some embodiments, two or more unique molecular identifiers are used to identify a heterologous gene effector, for example, one sequence can be used that is shared between heterologous gene effectors that are derived from the same source protein, class of proteins, or sub-library, and a second unique molecular identifier is used to distinguish between the distinct sequences, e.g., tiled segments of the protein that are encoded by different members of a library. In some embodiments, one set of unique molecular identifiers is used to identify heterologous gene effectors, and a separate set of unique molecular identifiers is used to uniquely tag molecules generated while processing a sample for sequencing, e.g., for deduplication.

Unique molecular identifiers can be rationally designed in silico. Methods of designing unique molecular identifiers can comprise, for example, criteria for minimum pairwise Hamming distance between unique molecular identifiers, a maximum homopolymer length, lower and upper GC content limits, blacklisted sequences (e.g., based on contents of a library and/or genome), a Markov chain model, and/or hyper-parameter optimization by grid search.

In some embodiments, assay systems of the disclosure can be iterated, for example, for strong binary combinations of effectors, screens can be performed to identify ternary complexes. In this scenario, stable cell lines could be generated with GA1-dCas-binary effector, and GID1-tagged individual effectors could be introduced.

In some embodiments, a polypeptide that is part of a complex of the disclosure (e.g., a heterologous gene effector, guide moiety, fusion protein etc.) is fused to a tag, such as a purification tag or epitope tag. Examples of tags include glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, hemagglutinin (HA), nus, Softag 1, Softag 3, Strep, SBP, Glu-Glu, HSV, KT3, S, SI, T7, V5, VSV-G, histidine (His), biotin carboxyl carrier protein (BCCP), and calmodulin.

In some embodiments, a polypeptide that is part of a complex of the disclosure (e.g., a heterologous gene effector, guide moiety, nuclease, fusion protein etc.) is fused to a degron to allow for temporal control of dCas9-effector expression, for example, a mini auxin-inducible degron (mAID; Yesbolatova et al., Nature Communications 2020). In some embodiments, a reporter cell line is generated. The cell line can be engineered to express OsTIRI. TIR (transport inhibitor 1 protein) is a subunit of a ubiquitin ligase complex from Oryza saliva that interacts with the AID in the presence of auxin. This interaction facilitates the ubiquitination and degradation of the AID-tagged protein, allowing the dCas9-effector to be degraded upon treating with auxin. An OsTIRI expression construct can be integrated into a safe harbor site (e.g., AAVS1) for consistent expression levels in screening assays. The OsTIRlconstruct can also be also modified to express a guide RNA that will target the dCas9 to a genomic locus (e.g., promoter) of interest in a given screening assay, or the guide RNA can be introduced separately.

In some embodiments, a polypeptide that is part of a complex of the disclosure (e.g., a heterologous gene effector, guide moiety, fusion protein etc.) is fused to a reporter gene, such as a fluorescent or luminescent protein. In some embodiments, a polypeptide that is part of a complex of the disclosure (e.g., a heterologous gene effector or guide moiety) is co-expressed with a reporter gene, e.g., co-expressed from an expression construct separated by an IRES. Non-limiting examples of reporter genes include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g. eBFP, eBFP2, Azurite, mKalamal, GFPuv, Sapphire, T-sapphire), cyan fluorescent proteins (e.g. eCFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (mOrange, mKO, Kusabira-Orange, Monomeric Kusabira-Orange, mTangerine, tdTomato), and any other suitable fluorescent protein.

In some embodiments, a complex or a heterologous gene effector identified by methods of the disclosure effects a desirable change in expression of a target gene (e.g., target endogenous gene) that is specific to a particular subject. In some embodiments, a complex or a heterologous gene effector identified by methods of the disclosure effects a desirable change in expression of a target gene that is applicable to two or more subjects. In some embodiments, a complex or a heterologous gene effector identified by methods of the disclosure effects a desirable change in expression of a target gene that is applicable to a class of subjects, for example, mammalian subjects, human subjects, male subjects, female subjects, subjects in a given age range, subjects with a similar disease or condition (e.g., having a disease with a genetic basis and/or a disease that is impacted by an expression level of one or more endogenous genes, including target genes or genes with related or opposing functions). In some embodiments, a complex or a heterologous gene effector identified by methods of the disclosure effects a desirable change in expression of a target gene that is broadly applicable to a wide variety subjects, for example, elicits an expression level of a target gene that is above or below a certain threshold for multiple subjects when introduced to the subject's cells using suitable methods.

In some embodiments, compositions and methods of the disclosure can be used to establish inducible and reversible disease models to understand disease mechanism.

In some embodiments, compositions and methods of the disclosure can be used to identify and/or test potential therapeutic agents or therapeutic agents for diseases that comprise aberrant expression of one or more particular genes.

In some embodiments, compositions and methods of the disclosure can be used to control cell differentiation by modulating expression of key drivers of cell fate and lineage commitment, for example, to induce differentiation into a particular cell type or de-differentiation into a cell type that is capable of differentiation down multiple pathways, such as a stem cell.

The disclosure further encompasses nucleic acids encoding any one or more of the elements disclosed herein, for example, heterologous gene effector(s), guide moieties, guide nucleic acids, complexes, oligomerization (e.g., heterodimerization) domains, combinations thereof, and libraries comprising multiples of the same.

The disclosure further encompasses kits comprising one or more of the elements disclosed herein, for example, heterologous gene effector(s), guide moieties, guide nucleic acids, complexes, oligomerization (e.g., heterodimerization) domains, combinations thereof, and libraries comprising multiples of the same, and nucleic acids encoding the same. Kits can further comprise, for example, instructions for use.

### Reporter Expression Vector

The disclosure provides a reporter expression vector that can be utilized, for example, in assay systems and methods disclosed herein. The reporter expression vector can be used, for example, in an engineered synthetic reporter (ESR) system or cell line described herein.

In some embodiments, the present disclosure provides an expression vector (e.g., a heterologous expression vector) comprising at least one polynucleotide sequence (e.g., one or more heterologous polynucleotide sequences) that can be targeted by any of the heterologous gene effector and/or the guide nucleic acid sequence (e.g., a complex comprising (i) a Cas protein coupled to a transcriptional/chromatin regulator and (ii) a guide RNA). The at least one polynucleotide sequence can be operatively coupled to a target gene (e.g., disposed upstream and adjacent to regulatory sequence, such as a promoter of the target gene), such that targeting of the at least one polynucleotide sequence by any of the heterologous gene effector and/or the guide nucleic acid sequence can modulate expression of the target gene. The at least one polynucleotide sequence can be a synthetic sequence that is not normally present in a cell (e.g., a mammalian cell), such to minimize (e.g., avoid) off-target effect(s), such as when screening for heterologous gene effector domain(s) and complex(es) thereof that exhibit desirable properties for modulating expression of the target gene. Thus, additional aspects of the present disclosure provide various embodiments of such expression vector, cells comprising thereof, and methods of use thereof.

The expression vector can comprise a heterologous polynucleotide sequence (e.g., a single heterologous polynucleotide sequence). Alternatively or additionally, the expression vector can comprise a plurality of heterologous polynucleotide sequences. The expression vector can comprise at least or up to about 2 heterologous polynucleotide sequences, at least or up to about 3 heterologous polynucleotide sequences, at least or up to about 4 heterologous polynucleotide sequences, at least or up to about 5 heterologous polynucleotide sequences, at least or up to about 6 heterologous polynucleotide sequences, at least or up to about 7 heterologous polynucleotide sequences, at least or up to about 8 heterologous polynucleotide sequences, at least or up to about 9 heterologous polynucleotide sequences, at least or up to about 10 heterologous polynucleotide sequences, at least or up to about 15 heterologous polynucleotide sequences, at least or up to about 20 heterologous polynucleotide sequences, at least or up to about 30 heterologous polynucleotide sequences, at least or up to about 40 heterologous polynucleotide sequences, or at least or up to about 50 heterologous polynucleotide sequences.

Each of the plurality of heterologous polynucleotide sequences of the expression vector can be substantially the same. In some embodiments, at least some of (e.g., each of) the plurality of heterologous polynucleotide sequences can be different from each other (e.g., by at least or up to about 1 nucleobase, at least or up to about 2 nucleobases, at least or up to about 3 nucleobases, at least or up to about 4 nucleobases, at least or up to about 5 nucleobases, 6 nucleobases, at least or up to about 7 nucleobases, at least or up to about 8 nucleobases, at least or up to about 9 nucleobases, or at least or up to about 10 nucleobases, etc.).

At least two (e.g., each) of the plurality of heterologous polynucleotide sequences of the expression vector can be directly adjacent to each other (e.g., not separated by any other nucleobase). Alternatively, at least two (e.g., each) of the plurality of heterologous polynucleotide sequences of the expression vector can be separated by a spacer, such as one or more nucleobases (e.g., at least or up to about 1 nucleobase, at least or up to about 2 nucleobases, at least or up to about 3 nucleobases, at least or up to about 4 nucleobases, at least or up to about 5 nucleobases, at least or up to about 6 nucleobases, at least or up to about 7 nucleobases, at least or up to about 8 nucleobases, at least or up to about 9 nucleobases, at least or up to about 10 nucleobases, at least or up to about 15 nucleobases, at least or up to about 20 nucleobases, at least or up to about 30 nucleobases, at least or up to about 40 nucleobases, at least or up to about 50 nucleobases, at least or up to about 60 nucleobases, at least or up to about 70 nucleobases, at least or up to about 80 nucleobases, at least or up to about 90 nucleobases, or at least or up to about 100 nucleobases, etc.).

A heterologous polynucleotide sequence of the expression vector, as disclosed herein, can comprise (i) an endonuclease target sequence (e.g., a CRISPR/Cas protein target sequence) and/or (ii) one or more CRISPR protospacer adjacent motif (PAM) sequences. The heterologous polynucleotide sequence can comprise a single PAM sequence. Alternatively, the heterologous polynucleotide sequence can comprise a plurality of PAM sequences (e.g., at least or up to about 2 PAM sequences, at least or up to about 3 PAM sequences, at least or up to about 4 PAM sequences, at least or up to about 5 PAM sequences, at least or up to about 6 PAM sequences, at least or up to about 7 PAM sequences, at least or up to about 8 PAM sequences, at least or up to about 9 PAM sequences, or at least or up to about 10 PAM sequences). The plurality of PAM sequences can be identical. In some embodiments, two or more of the plurality of PAM sequences can be different, e.g., a first PAM sequence can be for a first type of CRISPR/Cas protein (e.g., Cas9, such as SpCas9 and/or SaCas9) and a second PAM sequence can be for a second type of CRISPR/Cas protein (e.g., Cas12a). At least two of the plurality of PAM sequences may not overlap with each other (e.g., Cas12a PAM and Cas9 PAM). Alternatively, some of the plurality of PAM sequences may overlap with each other (e.g., SpCas9 PAM and SaCas9 PAM).

A distance between two PAM sequences as disclosed herein can be at least or up to about 1 nucleobase, at least or up to about 5 nucleobases, at least or up to about 10 nucleobases, at least or up to about 15 nucleobases, at least or up to about 20 nucleobases, at least or up to about 25 nucleobases, at least or up to about 30 nucleobases, at least or up to about 35 nucleobases, at least or up to about 40 nucleobases, at least or up to about 45 nucleobases, at least or up to about 50 nucleobases, at least or up to about 55 nucleobases, at least or up to about 60 nucleobases, at least or up to about 70 nucleobases, at least or up to about 80 nucleobases, at least or up to about 90 nucleobases, or at least or up to about 100 nucleobases. In some cases, at least two PAM sequences can be disposed on an end (e.g., the 5′ end or the 3′ end) of the heterologous polynucleotide sequence. At least two PAM sequences can be disposed on opposite ends of the heterologous polynucleotide sequence (e.g., the heterologous polynucleotide sequence can be flanked by two different PAM sequences).

The endonuclease target sequence (e.g., targeted by a guide RNA molecule of a Cas/guide RNA complex) of the heterologous polynucleotide sequence, as disclosed herein, can be at least or up to about 10 nucleobases, at last or up to about 15 nucleobases, at last or up to about 20 nucleobases, at last or up to about 25 nucleobases, at last or up to about 30 nucleobases, at last or up to about 35 nucleobases, at last or up to about 40 nucleobases, at last or up to about 45 nucleobases, at last or up to about 50 nucleobases, at last or up to about 60 nucleobases, at last or up to about 70 nucleobases, at last or up to about 80 nucleobases, at last or up to about 90 nucleobases, or at last or up to about 100 nucleobases.

The endonuclease target sequence (e.g., targeted by a guide RNA molecule of a Cas/guide RNA complex) of the heterologous polynucleotide sequence, as disclosed herein, can comprise a plurality of polynucleotide sequences that are derived from different sources. Non-limiting examples of the different sources can be an exon and an intron of the same gene, different genes, different chromosomes (e.g., different human chromosomes), etc. Different chromosomes can be from chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, chromosome X, and/or chromosome Y. In some examples, a polynucleotide sequence of the plurality of polynucleotide sequences can be derived from a gene encoding Cluster of Differentiation (CD) protein, such as CD1a, CD2, CD3, CD4, CD5, CD7, CD8, CD10, CD15, CD20, CD23, CD30, CD31, CD33, CD34, CD42b, CD43, CD45, CD45 RO, CD56, CD57, CD61, CD68, CD71, CD79a, CD99, CD103, CD117, CD138, CD163, etc.

The plurality of polynucleotide sequences can comprise at least or up to about 2 polynucleotide sequences, at least or up to about 3 polynucleotide sequences, at least or up to about 4 polynucleotide sequences, at least or up to about 5 polynucleotide sequences, at least or up to about 6 polynucleotide sequences, at least or up to about 7 polynucleotide sequences, at least or up to about 8 polynucleotide sequences, at least or up to about 9 polynucleotide sequences, or at least or up to about 10 polynucleotide sequences. Sizes (e.g., lengths) of the plurality of polynucleotide sequences can be the same. Alternatively, the sizes of the plurality of polynucleotide sequences can be different. Sizes of two polynucleotide sequences of the plurality of polynucleotide sequences can be different by at least or up to about 2 polynucleotides, at least or up to about 3 polynucleotides, at least or up to about 4 polynucleotides, at least or up to about 5 polynucleotides, at least or up to about 6 polynucleotides, at least or up to about 7 polynucleotides, at least or up to about 8 polynucleotides, at least or up to about 9 polynucleotides, or at least or up to about 10 polynucleotides.

The expression vector as disclosed herein can comprise an Upstream Activating Sequence (UAS) that is downstream of one or more heterologous polynucleotide sequence (e.g., each heterologous polynucleotide sequence) as disclosed herein. The UAS can be a non-human UAS. The UAS can be derived from a promoter (e.g., GAL4 promoter). Non-limiting examples of such promoter can include U6, H1, CYC1, HIS3, GAL1, GAL4, GAL10, ADH1, PGK, PHO5, GAPDH, T7, CMV, SV40 and EF1a. The UAS can be upstream of, for example, a promoter, and/or a target gene for which expression is to be regulated by the reporter expression vector, such as a reporter gene.

The expression vector as disclosed herein can comprise a promoter. In some cases, one or more heterologous polynucleotide sequences can be upstream of the promoter. In some cases, one or more heterologous polynucleotide sequences can be downstream of the promoter. In some cases, the promoter can be a strong constitutive promoter (e.g., EF1a). In some cases, the promoter can be a weak minimal viral promoter (e.g., CMV). The promoter can control expression of a target gene, e.g., the target gene can be under regulatory control of the promoter.

A heterologous polynucleotide sequence of the expression vector, as disclosed herein, can exhibit at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99, or substantially about 100% sequence identity or sequence similarity to any one or more of SEQ ID NOs: 49334-49341 or 49344-49352, as provided in TABLE 3. A heterologous polynucleotide sequence of the expression vector, as disclosed herein, can be joined or operatively coupled to a promoter that exhibits at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99, or substantially about 100% sequence identity or sequence similarity to any one of SEQ ID NOs: 49346, 49347, 49351, or 49352.

TABLE 3

| SEQ | | |
|---|---|---|
| ID | | |
| NO: | Description | Sequence |
| 49334 | Heterologous | GTTGTTCTAAACGCTCTGAG |
| | guide target | |
| 49335 | Cas12a PAM- | tttaGTTGTTCTAAACGCTCTGAG |
| | heterologous | |
| | guide target | |
| 49336 | Heterologous | GTTGTTCTAAACGCTCTGAGcgg |
| | guide target- | |
| | SpCas9 PAM | |
| 49337 | Heterologous | GTTGTTCTAAACGCTCTGAGcggag |
| | guide target- | |
| | SpCas9 PAM- | |
| | SaCas9 PAM | |
| 49338 | Cas12a PAM- | tttaGTTGTTCTAAACGCTCTGAGcggag |
| | heterologous | |
| | guide target- | |
| | SpCas9 PAM- | |
| | SaCas9 PAM | |
| 49339 | Guide RNA | tttaGTTGTTCTAAACGCTCTGAGcgg |
| | targeting | |
| | sequence | |
| 49340 | Guide RNA | tttaGTTGTTCTAAACGCTCTGAGcggagtactgtcctccg |
| | targeting | |
| | sequence with | |
| | UAS | |
| 49341 | UAS | Cggagtactgtcctccg |
| 49344 | Single ESR | GATCCTTTAGTTGTTCTAAACGCTCTGAGCGGAGTACTG |
| | repeat | TCCTCCGAGA |
| 49345 | 7 copies of ESR | GATCCTTTAGTTGTTCTAAACGCTCTGAGCGGAGTACTG |
| | | TCCTCCGAGAGATCCTTTAGTTGTTCTAAACGCTCTGAG |
| | | CGGAGTACTGTCCTCCGAGAGATCCTTTAGTTGTTCTAA |
| | | ACGCTCTGAGCGGAGTACTGTCCTCCGAGAGATCCTTTA |
| | | GTTGTTCTAAACGCTCTGAGCGGAGTACTGTCCTCCGAG |
| | | AGATCCTTTAGTTGTTCTAAACGCTCTGAGCGGAGTACT |
| | | GTCCTCCGAGAGATCCTTTAGTTGTTCTAAACGCTCTGA |
| | | GCGGAGTACTGTCCTCCGAGAGATCCTTTAGTTGTTCTA |
| | | AACGCTCTGAGCGGAGTACTGTCCTCCGAGAGATCCAT |
| | | TAGGCGGCCGCGTGGATAACCGTATTACCGCCATGCAT |
| 49346 | miniCMV | GTAGGCGTGTACGGTGGGAGGTCTATATAAGCAGAGCT |
| | promoter | |
| 49347 | EF1a promoter | GTGCCCGTCAGTGGGCAGAGCGCACATCGCCCACAGTC |
| | | CCCGAGAAGTTGGGGGGAGGGGTCGGCAATTGAACCG |
| | | GTGCCTAGAGAAGGTGGCGCGGGGTAAACTGGGAAAG |
| | | TGATGTCGTGTACTGGCTCCGCCTTTTTCCCGAGGGTGG |
| | | GGGAGAACCGTATATAAGTGCAGTAGTCGCCGTGAACG |
| | | TTCTTTTTCGCAACGGGTTTGCCGCCAGAACACAGGTAA |
| | | GTGCCGTGTGTGGTTCCCGCGGGCCTGGCCTCTTTACGG |
| | | GTTATGGCCCTTGCGTGCCTTGAATTACTTCCACCTGGC |
| | | TGCAGTACGTGATTCTTGATCCCGAGCTTCGGGTTGGAA |
| | | GTGGGTGGGAGAGTTCGAGGCCTTGCGCTTAAGGAGCC |
| | | CCTTCGCCTCGTGCTTGAGTTGAGGCCTGGCCTGGGCGC |
| | | TGGGGCCGCCGCGTGCGAATCTGGTGGCACCTTCGCGC |
| | | CTGTCTCGCTGCTTTCGATAAGTCTCTAGCCATTTAAAA |
| | | TTTTTGATGACCTGCTGCGACGCTTTTTTTCTGGCAAGA |
| | | TAGTCTTGTAAATGCGGGCCAAGATCTGCACACTGGTA |
| | | TTTCGGTTTTTGGGGCCGCGGGCGGCGACGGGGCCCGT |
| | | GCGTCCCAGCGCACATGTTCGGCGAGGCGGGGCCTGCG |
| | | AGCGCGGCCACCGAGAATCGGACGGGGGTAGTCTCAAG |
| | | CTGGCCGGCCTGCTCTGGTGCCTGGCCTCGCGCCGCCGT |
| | | GTATCGCCCCGCCCTGGGCGGCAAGGCTGGCCCGGTCG |
| | | GCACCAGTTGCGTGAGCGGAAAGATGGCCGCTTCCCGG |
| | | CCCTGCTGCAGGGAGCTCAAAATGGAGGACGCGGCGCT |
| | | CGGGAGAGCGGGCGGGTGAGTCACCCACACAAAGGAA |
| | | AAGGGCCTTTCCGTCCTCAGCCGTCGCTTCATGTGACTC |
| | | CACGGAGTACCGGGCGCCGTCCAGGCACCTCGATTAGT |
| | | TCTCGcGCTTTTGGAGTACGTCGTCTTTAGGTTGGGGGG |
| | | AGGGGTTTTATGCGATGGAGTTTCCCCACACTGAGTGG |
| | | GTGGAGACTGAAGTTAGGCCAGCTTGGCACTTGATGTA |
| | | ATTCTCCTTGGAATTTGCCCTTTTTGAGTTTGGATCTTGG |
| | | TTCATT |
| 49348 | Single TRE3G | GAGTTTACTCCCTATCAGTGATAGAGAACGTATGAA |
| | repeat | |
| 49349 | Cas9 spacer | TATCAGTGATAGAGAACGTA |
| 49350 | CasMINI/Cas12a | CTCCCTATCAGTGATAGAGAACG |
| | spacer | |
| 49351 | TRE3GS | GAGTTTACTCCCTATCAGTGATAGAGAACGTATGAAGA |
| | promoter | GTTTACTCCCTATCAGTGATAGAGAACGTATGCAGACTT |
| | | TACTCCCTATCAGTGATAGAGAACGTATAAGGAGTTTA |
| | | CTCCCTATCAGTGATAGAGAACGTATGACCAGTTTACTC |
| | | CCTATCAGTGATAGAGAACGTATCTACAGTTTACTCCCT |
| | | ATCAGTGATAGAGAACGTATATCCAGTTTACTCCCTATC |
| | | AGTGATAGAGAACGTATAAGCTTTGCTTATGTAAACCA |
| | | GGGCGCCTATAAAAGAGTGCTGATTTTTTGAGTAAACTT |
| | | CAATTCCACAACACTTTTGTCTTATACCAACTTTCCGTA |
| | | CCACTTCCTACCCTCGTAAA |
| 49352 | modified | CTTTGCTTATGTAAACCAGGGCGCCTATAAAAGAGTGC |
| | miniCMV | T |
| | promoter | |

Another aspect of the present disclosure provides cells (e.g., reporter cell lines) comprising any of the expression vectors disclosed herein, methods of producing the cells, and methods of using the cells to, for example, screen for identifying one or more lead heterologous gene effectors of the library as disclosed herein. The expression vector can be integrated into one or more chromosomes (e.g., nuclear chromosomes) of the cells. Alternatively, the expression vector may not be integrated into a chromosome (e.g., may be achromosomal).

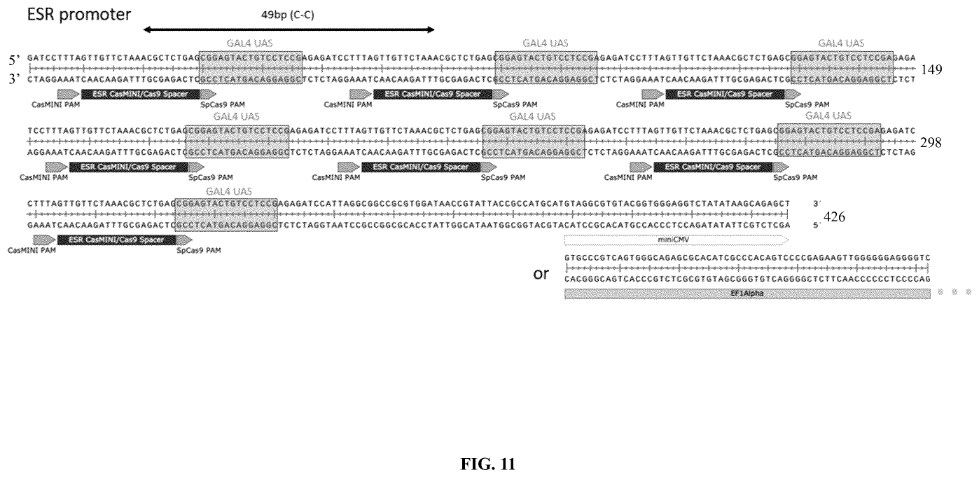

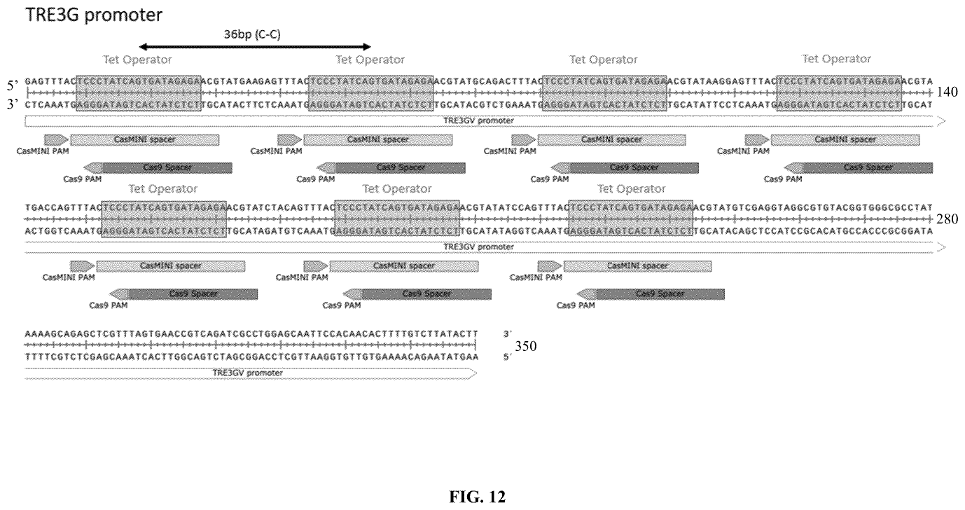

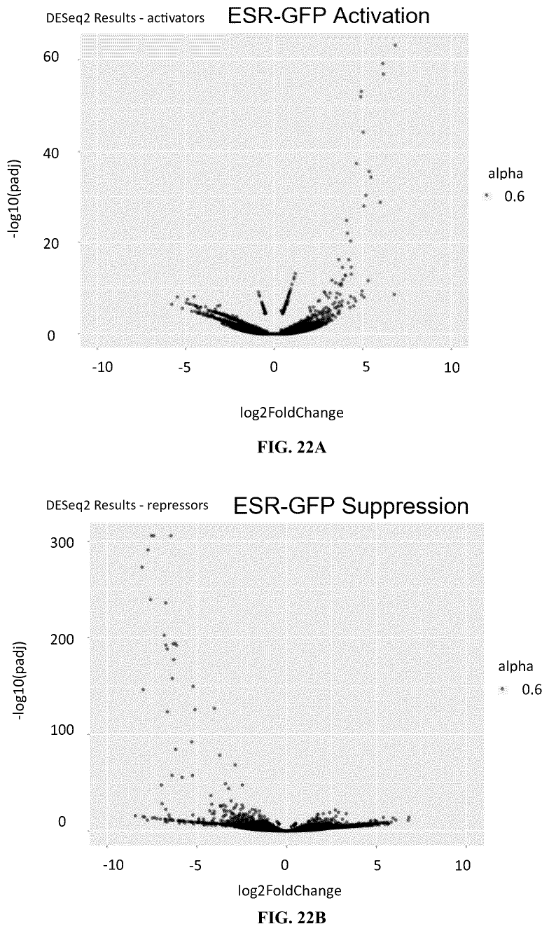

A population of cells comprising the expression vector (e.g., see FIG. 11) disclosed herein can exhibit a narrower distribution of target gene-positive cells as compared to a control population of cells comprising a control expression vector (e.g., see FIG. 12), by at least or up to about 1 percent (%), at least or up to about 2%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 90%, at least or up to about 95%, at least or up to about 100%, at least or up to about 0.1-fold, at least or up to about 0.2-fold, at least or up to about 0.5-fold, at least or up to about 1-fold, at least or up to about 1.5-fold, at least or up to about 2-fold, at least or up to about 3-fold, at least or up to about 4-fold, at least or up to about 5-fold, or at least or up to about 10-fold (see FIG. 19). The distribution can be, for example, a range, interquartile range, or range of the from about the 5th to 95th percentile of fluorescence intensities for expression of a reporter gene by cells in a population as determined by flow cytometry.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure.

### Example 1: Human Epigenetic Effector Screen

This example describes identification and characterization of novel gene effectors (e.g., domains that modulate gene expression) from the human nuclear proteome.

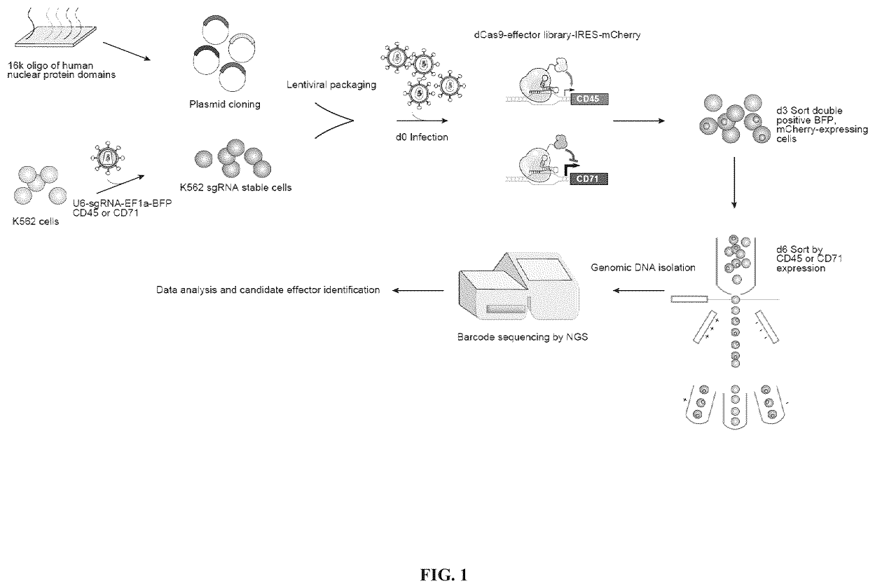

A high throughput screen is conducted utilizing a library of complexes that contain a nuclease-dead Cas9 (dCas9) and a heterologous gene effector candidate peptide sequence. Each heterologous gene effector candidate peptide sequence is fused to dCas9, recruited to target gene regulatory sequences (e.g., promoters) in human cells, and the effect on target gene expression determined. An overview of an illustrative effector screen design is provided in FIG. 1.

### Library Design

The heterologous gene effector candidate peptide sequences are designed to cover protein sequences from the human nuclear proteome. An analysis of human proteins experimentally determined to be localized (e.g., exclusively localized) to the nucleus (ProteinAtlas.org) is conducted, resulting in a shortlist of 549 proteins. The heterologous gene effector candidate peptide sequences are generated from synthetic DNA oligonucleotides 300 nucleotides in length, of which 255 nucleotides are target specific. The 5′ and 3′ ends of each oligo contains sequences complementary to the destination vector (p-dCas9), with the 3′ 15 nucleotide overlap comprising part of the Illumina Read 1 Primer. The oligonucleotides also contain unique molecular identifiers (e.g., “barcodes”) for each heterologous gene effector candidate peptide sequence. This design allows for cloning of the library using NEB HiFi cloning, and for downstream generation of Illumina Hiseq-compatible NGS libraries. For each target gene, oligos are designed such that they are tiled across the coding nucleotide sequence of the gene from 5′ to 3′, with overlaps of 129 nucleotides.

In total 16139 oligos are designed in this initial effector library, including positive and negative controls. The following provides an illustrative example of an oligonucleotide of this example, annotated as follows:

vector overlap 1 (SEQ ID NO: 11)-target sequence (SEQ ID
NO: 12)-[stop codon; SEQ ID NO: 13]-unique molecular identifier (SEQ ID NO: 14)-
vector overlap 2 (SEQ ID NO: 15; (Illumina Read 1 partial):
GCAGTGGTGGACATAATGTCTATGCCAGAACATGTTTTAACGAGTGAATCC
ATGCATGTGTGTGACATTGGACATGTTGAACATATGGTGCATGATAGTGTAGTGGAA
GCAGAAATCATTACTGATCCTCTGACGAGTGACATAGTTTCAGAAGAAGTATTGGTA
GCAGACTGTGCCCCTGAAGCAGTCATAGATGCCAGCGGGATCTCAGTGGACCAGCA
AGATAATGACAAAGCCAGCTGTGAGGACTACCTAATGATTTCGTTGGAT[TAG]AGG
GGTTCACTATACGTTACTGGCCGA

Oligos used in libraries of the disclosure can utilize a similar format to associate a unique molecular identifier to each target sequence, and to provide vector overlap sequences for cloning.

Library cloning

The oligonucleotides are obtained at a yield of >0.2 fmol per oligo (e.g., several hundred nanograms of lyophilized DNA). The oligos are resuspended in 10 mM Tris pH 8.0 to a stock concentration of 20ng/μL. Stock solutions can be maintained at −20° C.

PCR is conducted to convert ssDNA to dsDNA, ready for NEBuilder HiFi cloning. A KAPA HiFi HotStart PCR Kit (Catalog #KK2502) is used, with approximately 12-14 cycles.

Table 4 provides illustrative PCR reaction components for library amplification. 400 μL reactions are split amount 8 PCR tubes, with 50 μL each.

| | | Final |
|---|---|---|
| Component | Volume | concentration |
| Library Amp F primer at 100 μM | 2 μl | 0.5 μM |
| Library Amp R primer at 100 μM | 2 μl | 0.5 μM |
| Twist oligo pool at 4 ng/μl | 20 μl | 0.2 ng/μl |
| dH2O | 176 μl | — |
| Q5 Hot Start 2× Master Mix | 200 μl | 1× |

Table 5 provides illustrative PCR conditions.

| Temperature | Time | Cycles |
|---|---|---|
| 98° C. | 30 s | 1× |
| 98° C. | 10 s | 12-14× |
| 59° C. | 30 s | |
| 72° C. | 20 s | |
| 72° C. | 2 m | 1× |

PCR products are cleaned up via column purification.

The overlapping sequences present at the 5′ and 3′ ends of each oligo permits cloning into the destination vector (e.g., via NEB HiFi).

A destination vector (p-dCas9) is generated by modifying a lentiviral backbone based on pSLQ6604. The destination vector is designed to allow fusion of the heterologous gene effector to the C-terminus of dCas9 via a short flexible GS linker. The destination vector is generated by direct synthesis (e.g., GenScript) or by assembly (e.g., HiFI PCR assembly using three synthetic gBlock fragments from IDT, with subsequent ligation into EcoRI NotI-linearized pSLQ6604).

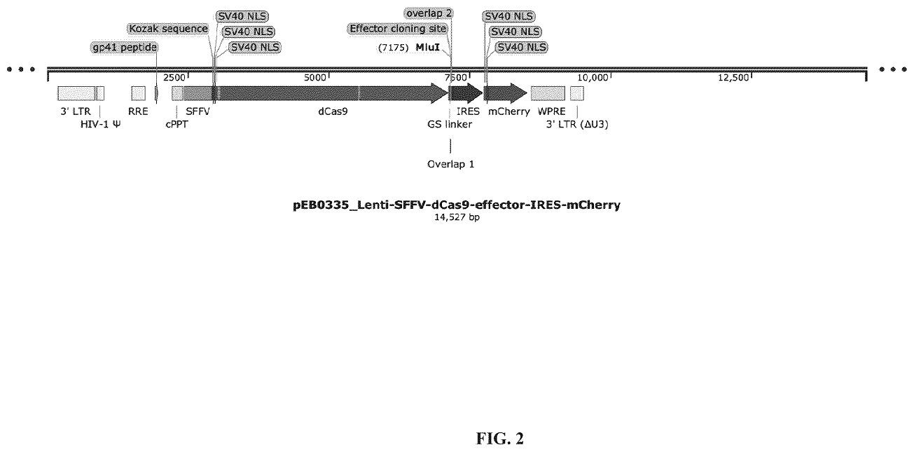

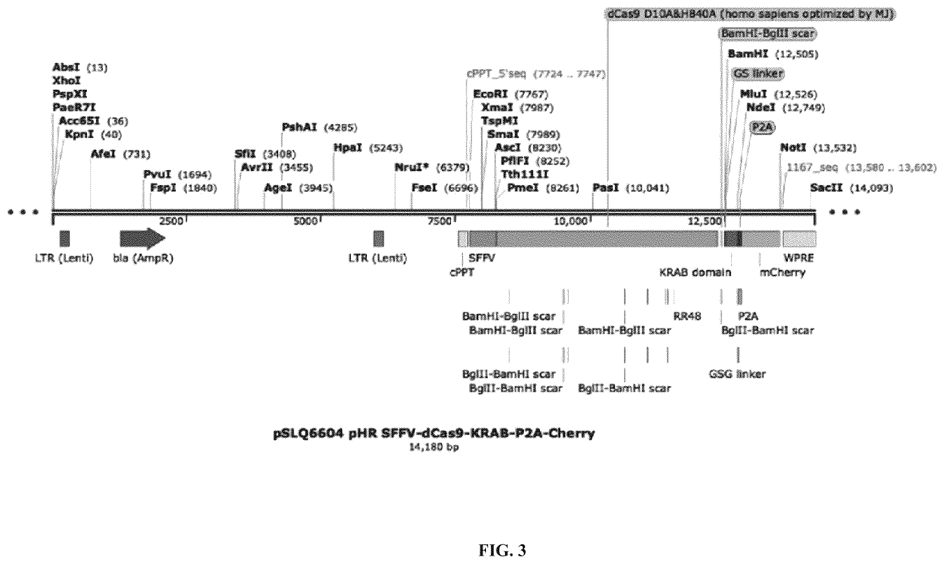

The starting destination vector, illustrated in FIG. 2, is linearized (e.g., via digestion with EcoRI (high fidelity) and NotI (high fidelity) restriction enzymes. An insert sequence generated from the three synthetic gBlock fragments is inserted via HiFi assembly to generate p-dCas9, as illustrated in FIG. 3.

The p-dCas9 destination vector is linearized (e.g., via digestion with MluI (high fidelity)), and an insert encoding the heterologous gene effector sequence is added into the vector using HiFi assembly, with a ratio of insert to vector of approximately 10:1.

Illustrative activator screens

The library is used to identify heterologous gene effectors that activate or increase expression of target genes. For each screen, a suitable cell type/cell line and target gene or target gene regulatory sequence is selected. A guide RNA is used to direct the dCas9-effector to a regulatory sequence (e.g., promoter) of the endogenous gene. The guide RNA can be a validated guide RNA. The library includes positive control oligonucleotides (e.g., tiled across the P300, TET1, TET2, TET3, and HSF1 genes).

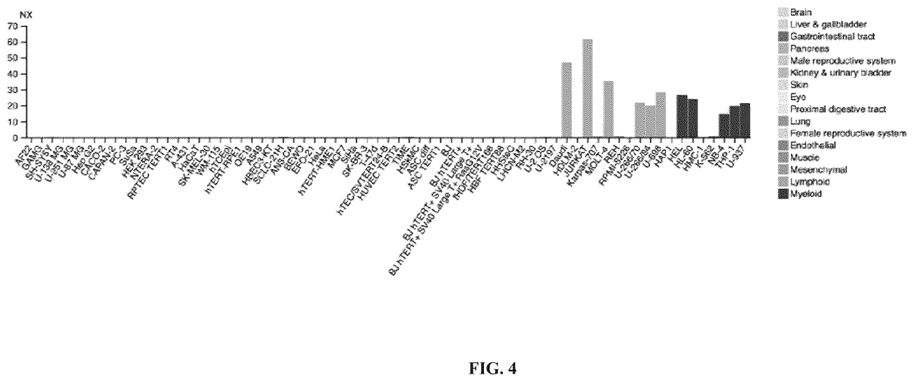

Target genes encoding cell surface proteins that are expressed at low levels in a given cell type can be used. For example, cell lines that express low levels of CD45 are illustrated in FIG. 4.

In one iteration, K562 cells are used, with CD45 as a target gene. K562 cells express low levels of CD45. A guide RNA is used to direct the dCas9-effector to a regulatory sequence (e.g., promoter) of CD45.

In one iteration, K562 cells are used, with CDB1 as a target gene. K562 cells express low levels of CDB1. A guide RNA is used to direct the dCas9-effector to a regulatory sequence (e.g., promoter) of CDB1.

Cells are transduced with vectors (e.g., lentiviral particles) to induce expression of complexes of the library. A multiplicity of infection is selected such that no more than one candidate heterologous gene effector is expressed in a transduced cell. Transduced cells are sorted based on reporter gene (e.g., mCherry) expression to minimize the number of background cells in the screen that do not express a dCas9-effector. Cells are then transduced with vectors (e.g., lentiviral particles at a high multiplicity of infection) to induce expression of the guide RNAs specific to the screen (e.g., guide RNA targeting a CD45 promoter). Transduced cells are maintained for up to about five days in culture.

Cells that express higher levels of the target gene can be identified, for example, by sorting cells based on expression of the target gene (e.g., via fluorescent activated cell sorting and/or magnetic activated cell sorting).

Genomic DNA is extracted from the cells, and the integrated effector cassette is isolated by PCR, and used to generate libraries for HiSeq single end barcode sequencing. Gene effectors are identified that increase expression of the target gene.

The approach can also be adapted to screen combinations of heterologous gene effectors.

### Illustrative Repressor Screens

The library is used to identify heterologous gene effectors that reduce expression of target genes. For each screen, a suitable cell type/cell line and target gene or target gene regulatory sequence is selected. A guide RNA is used to direct the dCas9-effector to a regulatory sequence (e.g., promoter) of the endogenous gene. The guide RNA can be a validated guide RNA.

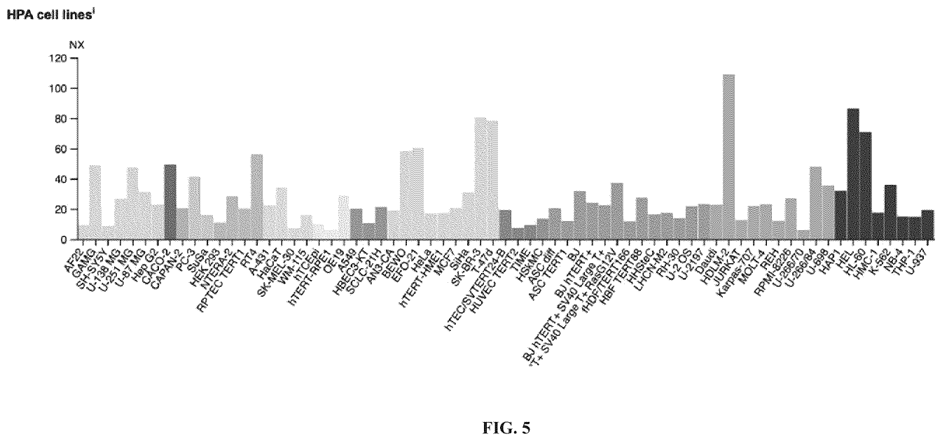

Target genes encoding cell surface proteins that are expressed at relatively high levels in a given cell type can be used. For example, cell lines that express high levels of the transferrin receptor CD71 are illustrated in FIG. 5.

In one iteration, K562 cells are used, with CD71 as a target gene. K562 cells express relatively high levels of CD71. A guide RNA is used to direct the dCas9-effector to a regulatory sequence (e.g., promoter) of CD71. The guide RNA can be encoded by, for example, GGACGCGCTAGTGTGAGTGC or CGATATCCCGACGCTCTGAG.

Cells are transduced with vectors (e.g., lentiviral particles) to induce expression of complexes of the library. A multiplicity of infection is selected such that no more than one candidate heterologous gene effector is expressed in a transduced cell. Transduced cells are sorted based on reporter gene (e.g., mCherry) expression to minimize the number of background cells in the screen that do not express a dCas9-effector. Cells are then transduced with vectors (e.g., lentiviral particles at a high multiplicity of infection) to induce expression of the guide RNAs specific to the screen (e.g., guide RNA targeting a CD71 promoter). Transduced cells are maintained for up to about five days in culture.

Cells that express lower levels of the target gene can be identified, for example, by sorting cells based on expression of the target gene (e.g., via fluorescent activated cell sorting and/or magnetic activated cell sorting).

Genomic DNA is extracted from the cells, and the integrated effector cassette is isolated by PCR, and used to generate libraries for HiSeq single end barcode sequencing. Gene effectors are identified that reduce expression of the target gene.

The approach can also be adapted to screen combinations of heterologous gene effectors.

### Example 2: Pilot Experiment Demonstrating Activation and Repression of Gene Expression by Complexes of the Disclosure

This example describes a pilot experiment demonstrating the ability of complexes of the disclosure to activate and repress expression of target genes, and the ability to detect upregulation and downregulation of target genes using methods of the disclosure.

A library of complexes that each contain a nuclease-dead Cas9 (dCas9) and a heterologous gene effector candidate peptide sequence was generated using methods described in Example 1. Synthetic DNA nucleotides encoding each heterologous gene effector and containing 5′ and 3′ cloning sequences were generated and cloned into the destination vector p-dCas9. A library of candidate effectors was pooled for the pilot screens. The library includes candidate transcriptional activators and candidate transcriptional repressors, including from genes that have been identified as modulating transcriptional activity (e.g., P300, TET1, TET2, TET3, HDAC3, HSF1 and ZIM3, MeCp2, DNMT3L, DNMT3a, DNMT3b, G9a, and EZH2).

A K562 cell line was used to test the impact of the complexes on gene expression. Cells were transduced with constructs encoding the candidate effectors to induce expression of complexes of the library. Cells were also transduced with lentiviral vectors to induce stable expression of guide RNAs to direct the dCas9-effectors to (i) a regulatory sequence of CD45 (activator screen, experimental condition), (ii) a regulatory sequence of CD71 (repressor screen, experimental condition), or (iii) GAL4 (controls). The transduced cells were sorted based on reporter gene expression to enrich for cells that express both the guide RNA and a dCas9-effector.

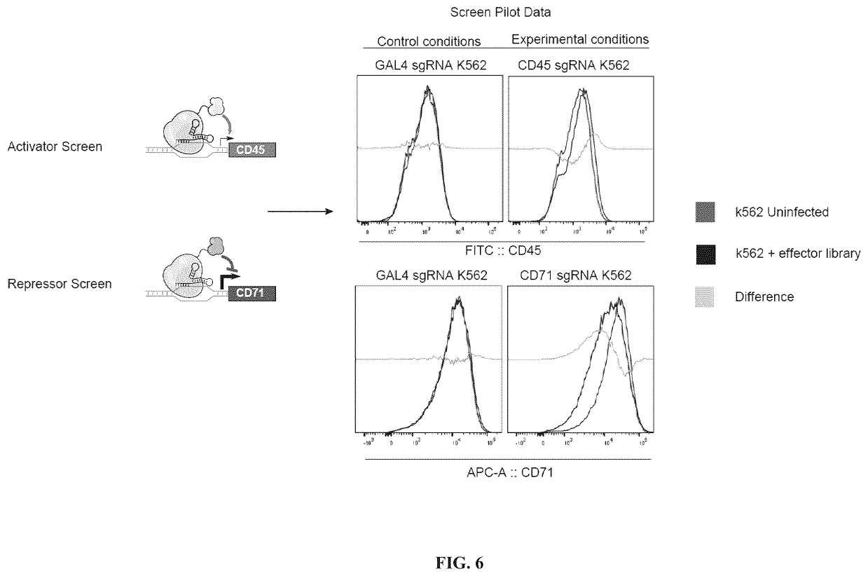

Transduced cells were maintained for up to about five days in culture, and expression of CD45 or CD71 was quantified via flow cytometry. Increased expression of CD45 was observed in experimental conditions in the activator screen (FIG. 6, top right panel), and reduced expression of CD71 was observed in experimental conditions in the repressor screen (FIG. 6, bottom right panel).

### Example 3: Viral Epigenetic Effector Screen Design

Heterologous gene effector candidate peptide sequences are designed to cover protein sequences from the viral genomes. Candidate sequences are selected based on:
(i) viral transcriptional regulators that have been experimentally validated by ChIP/ChIP-seq, EMSA, SELEX, reporter assays, binding assays, and/or crystal structures. Experimentally validated viral transcriptional regulators can represent viruses from, for example, any one or more of the families Adenoviridae, Arenaviridae, Bornaviridae, Coronaviridae, Filoviridae, Flaviviridae, Hepadnaviridae, Herpesviridae, Orthomyxoviridae, Papillomaviridae, Paramyxoviridae, Parvoviridae, Peribunyaviridae, Phenuiviridae, Pneumoviridae, Polyomaviridae, Poxviridae, Retroviridae, and Rhabdoviridae. Transcriptional regulators can be included from Liu et al., Human virus transcriptional regulators. Cell, 182(1), pp.22-37.
(ii) viral transcriptional regulators from human-bat shared viruses and viral proteins, such as viral families with documented zoonotic transmission from bats and/or viruses that have been demonstrated to function in human cells. Viral transcriptional regulators from human-bat shared viruses and viral proteins can represent viruses from, for example, any one or more of Flaviviridae, Lyssaviridae, Filoviridae, Paramyxoviridae, Orthomyxoviridae, Coronaviridae, Reoviridae, Togaviridae, Phenuviridae, and Hantaviridae. Sequences from Adenoviridae and poxviridae can be included, for example, due to a high degree of transcriptional regulator modularity in these viruses. Sequences can be obtained manually from the dBatVir database.
(iii) metagenomic virus genomes and viral proteins. Sequences can be obtained from, for example, data from studies evaluating the gut virome; genomes of Siphoviridae, podoviridae, and myoviridae that are abundant in human gut, contain transcriptional regulators, and occur in acidic environments; viral sequences detected in oceans; viral sequences detected in geothermal vents; sources that utilize structural data (e.g., X-ray, NMR) from public databases; records of archaea-tropic viruses (e.g., sulfolobus) from extreme environments (e.g., high-acid, high-temperature environments) that may be enriched for effectors with desirable properties such as acidic residues and persistent function due to evolutionary pressures favoring vertical versus horizontal transmission; and sequences manually obtained from public sources (e.g., NCBI).

Predictive filtering techniques can be applied to candidate sequences, for example, based on identification of suitable biophysical properties for core activator domains (e.g., down to 13 bp) through experiments in yeast; the presence of acidic, bulky hydrophobic, alpha helix, and/or negative charge; the presence of motif repeats that may influence duration of effect; and PADDLE-like convolutional neural network/transformer algorithms or similar predictive techniques.

### Example 4: Viral Epigenetic Effector Screen

This example describes identification and characterization of novel gene effectors (e.g., domains that modulate expression of human genes) from viral genomes.

A high throughput screen is conducted utilizing a library of complexes that each contain a nuclease-dead Cas9 (dCas9) and a heterologous gene effector candidate peptide sequence from a viral genome. The heterologous gene effector candidate peptide sequences (e.g., identified as in example 3) are fused to dCas9, recruited to target gene regulatory sequences (e.g., promoters) in human cells, and the effect on target gene expression determined (e.g., using techniques described for the human nuclear proteome screen in example 1).

27799 oligos are designed targeting approximately 3500 genes, plus approximately 3000 controls. Positive and negative controls are used as disclosed in Example 1. Viral transcriptional regulators with published experimental data are used as a benchmark for assay performance, and known activities are manually organized to guide data analysis.

Gene effectors are identified that enhance or reduce expression of the target gene.

The approach can also be adapted to screen combinations of heterologous gene effectors.

### Example 5: Combinatorial Epigenetic Effector Screen

This example describes identification and characterization of novel combinations of gene effectors that modulate gene expression.

A high throughput screen is conducted utilizing a library of complexes that contain a nuclease-dead Cas9 (dCas9) complexed with combinations of heterologous gene effector candidate peptide sequences. The screen is designed to allow a systematic and large scale survey of combinations of gene effectors (e.g., genetic/epigenetic factors) in human cells.

In a first approach, chromatin regulators (CRs) and transcriptional regulators (TRs) from diverse protein families are drawn from literature and databases. Combinations of the CRs and TRs are screened to identify novel combinations of factors that modulate (e.g., increase or reduce) expression of target genes. The screen allows identification of combinations of factors that may not interact in their in vivo contexts (e.g., due to cell type specificity, cofactor or complex requirements, etc.) but could nevertheless mediate epigenetic and/or transcriptional effects when orthogonally recruited to a locus of interest.

CR gene effectors include functional domains from various classes of histone and DNA modifying enzymes (e.g., DNMTs, HATs, HMTs, etc.). These are primarily of human origin, but may also include factors derived from plants or other species. The CR library is in the order of ˜100 candidate gene effectors.

TR gene effectors include transcriptional regulatory domains from various families of TFs (e.g. KRAB, p65, MED, GTFs, etc.). Similar to the CR library, these domains will include primarily human sequences, and similarly in the order of ˜100 factors, providing approximately 10,000 unique combinations of CR+TR.

In a second approach, gene effectors identified in examples 1-3 are screened. The approach allows identification of novel effector domains whose activity can be augmented when deployed in combination with each other or with other effector domains (e.g., effector domains from literature). Paired libraries are generated comprising effectors from the initial screens that achieve activation or repression above a certain threshold, as well as other activators and repressors. These libraries are then tested in a pairwise fashion to identify combinations of effectors exhibit desirable (e.g., synergistic) activity.

An inducible system is used that facilitates orthogonal recruitment of candidate effector domains and allows thousands of possible combinations to be tested in a targeted or an unbiased manner. The inducibility and reversibility of the system also allows the persistence of observed effects on transcriptional activation or repression to be evaluated.

The system utilizes inducible heterodimeric protein pairs derived from Arabidopsis thaliana (PYL1-ABI and GID1-GAI). By fusing one protein from each heterodimeric pair to dCas9 and the cognate proteins to effector domains, effector recruitment to dCas9 is achieved by addition of the inducer molecules (the plant hormones ABA and GA). Up to 80 amino acid gene effectors are fused to the heterodimerization domains.

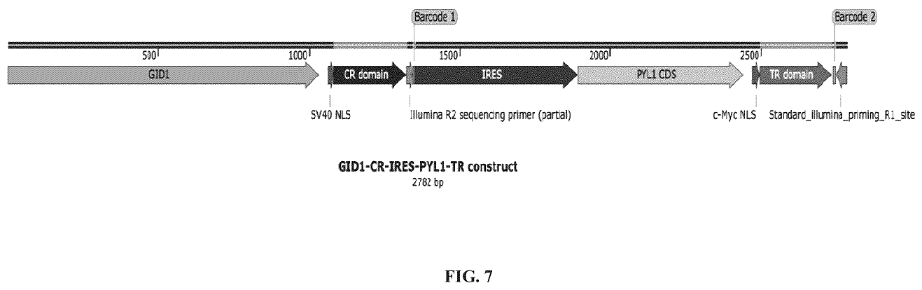

Each library element contains a unique molecular identifier (barcode), and the unique molecular identifiers are arranged in a way such that pair-end Illumina sequencing will deconvolute the combinations present in cells that pass selection thresholds. DNA encoding the up to 80 amino acid gene effector fragments and unique molecular identifiers is obtained in the form of approximately 300 nucleotide oligonucleotides. Lentiviral expression plasmids are generated, with single plasmids having a CR gene effector and a TR gene effector in-frame with GID1 and PYL1, respectively. An illustrative schematic of an expression construct for the combinatorial screen is provided in FIG. 7.

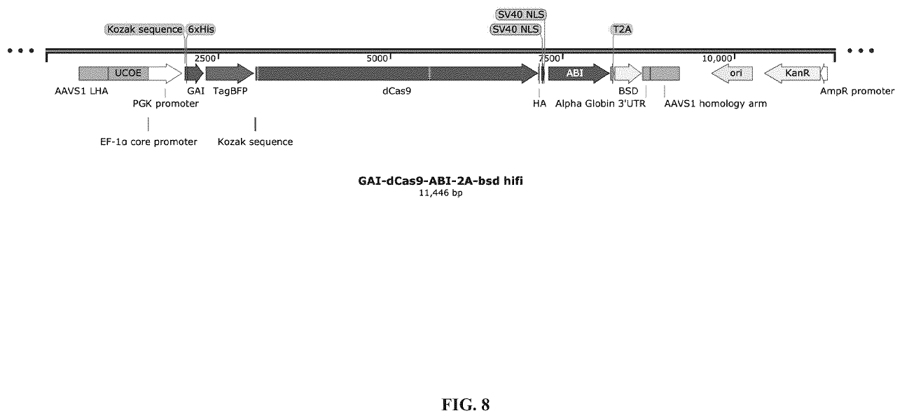

For compatibility with the screens described in examples 1-3, the same loci are targeted (CD45 for activation, CD71 for repression). A stable K562 cell line expressing GAI-dCas9-ABI is generated, for example, using the expression construct illustrated in FIG. 8.

The combinatorial libraries are transduced as lentiviral particles into K562 cells stably expressing GAI-dCas9-ABI. Transduced cells are exposed to ABA and GA at concentrations experimentally determined to induce the recruitment of PYL— and GID1—tagged candidate effector domains. The assembled complex of gene effectors and dCas9 is allowed to remain associated with the target gene locus (CD71 in repressor, and CD45 in activator screens) for up to 5 days before the withdrawal of the inducing hormones.

The activity of effector combinations is determined by sorting and collection of the desired population of cells at 5 day intervals post-induction for up to 30 days. The identities of candidate combinations is then determined by sequencing the unique molecular identifiers from DNA extracted from isolated cells at the indicated times.

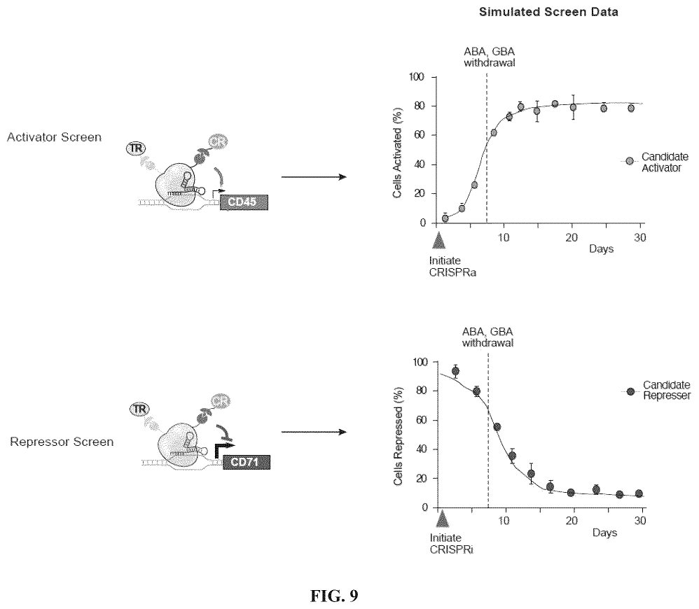

FIG. 9 illustrates modulation of CD45 and CD71 expression by complexes of the disclosure that comprise combinations of a transcriptional regulator and a chromatin regulator associated with dCas9. The top images show an illustrative activator screen for CD45, and the bottom images show an illustrative repressor screen for CD71. The graphs on the right illustrate repression of CD71 expression by a complex of the disclosure, and an increase in CD45 expression.

Combinations of gene effectors are identified that increase or repress expression of genes of interest.

Further iterations of the screen can be conducted with additional candidate gene effectors, and/or to identify ternary complexes. For example, stable lines of K562 cells can be generated with GA1-dCas-binary effector, and GID1-tagged individual effectors can be introduced.

### Example 6: Therapeutic Applications of the Systems and Methods Disclosed Herein

The systems and methods disclosed herein can be used, for example, to identify a novel gene effector complex. The novel gene effector complex can comprise a novel gene effector and/or a novel combination of a plurality of gene effectors. The novel gene effector identified, as disclosed herein, can be unique for a specific target gene, a specific cell type, a specific target disease, a specific subject, etc.

a. Same Cell Type, and Different Target Genes

The library of complexes, as disclosed herein, can be used to identify a novel gene effector complex for different target genes from the same cell type. Within the same cell type, a first novel gene effector complex identified to regulate (e.g., optimally upregulate or downregulate) expression or activity level of a first gene may be different than a second novel gene effector complex identified to regulate (e.g., optimally upregulate or downregulate) expression or activity level of a second gene (that is different than the first gene).

For example, within the same stem cell type, a first novel gene effector complex identified to optimally enhance expression or activity level of telomerase for inducing rejuvenation of the stem cell may be different than a second novel gene effector complex identified to optimally enhance expression or activity level of a transforming growth factor for tissue repair/regeneration.

b. Different Cell Types, and Same Target Gene

The library of complexes, as disclosed herein, can be used to identify novel gene effector complex for the same target gene but in different cell types. Even if the same target gene is utilized for the screening, a first novel gene effector complex identified to regulate (e.g., optimally upregulate or downregulate) expression or activity level of the target gene in a first cell type may be different than a second novel gene effector complex identified to regulate (e.g., optimally upregulate or downregulate) expression or activity level of the same target gene in a second cell type.

For example, given the same target gene, a first novel gene effector complex identified to optimally enhance expression or activity level of a gene encoding telomerase for inducing rejuvenation of a stem cell may be different than a second novel gene effector complex identified to optimally enhance expression or activity level of the same gene encoding the telomerase for inducing rejuvenation of a more differentiated cell (e.g., a skin cell, a muscle cell, a neuron, etc.).

In a different example, given the same target gene, a first novel gene effector complex identified to optimally enhance expression or activity level of a gene encoding telomerase for inducing rejuvenation of a first type of differentiated cell (e.g., a skin cell) may be different than a second novel gene effector complex identified to optimally enhance expression or activity level of the same gene encoding the telomerase for inducing rejuvenation of a second type of differentiated cell (e.g., a muscle cell).

c. Different Cell Types, and Different Target Genes

The library of complexes, as disclosed herein, can be used to identify novel gene effector complex for different target genes in different cell types. A first novel gene effector complex identified to regulate (e.g., optimally upregulate or downregulate) expression or activity level of a first target gene in a first cell type may be different than a second novel gene effector complex identified to regulate (e.g., optimally upregulate or downregulate) expression or activity level of a second target gene in a second cell type.

For example, a first novel gene effector complex identified to optimally enhance expression or activity level of a first gene encoding myoblast determination protein 1 (MyoD) in a muscle stem cell (or a muscle satellite cell) for inducing myogenesis may be different than a second novel gene effector complex identified to optimally enhance expression or activity level of a second gene encoding Runt-related transcription factor 2 (RUNX2) for inducing osteogenesis in a mesenchymal stem cell (MSC).

### d Different Subjects, and Same Target Gene

The library of complexes, as disclosed herein, can be used to identify novel gene effector complex for the same target gene, in the same target cell type, but from different target cell source. In some cases, the target cells of the same type may be from different subjects. The different subjects may be different by, for example, gender, age, condition (e.g., disease state), etc. A first novel gene effector complex identified to regulate (e.g., optimally upregulate or downregulate) expression or activity level of a target gene in a cell type from a first subject may be different than a second novel gene effector complex identified to regulate (e.g., optimally upregulate or downregulate) expression or activity level of the same target gene in the same cell type from a second subject that is different from the first subject.

For example, given the same target gene, a first novel gene effector complex identified to optimally enhance expression or activity level of a gene encoding telomerase for inducing rejuvenation of a stem cell type from a female subject may be different than a second novel gene effector complex identified to optimally enhance expression or activity level of the same gene encoding the telomerase in the same stem cell type from a male subject.

### Example 7: Design for Engineered Synthetic Reporter (ESR)

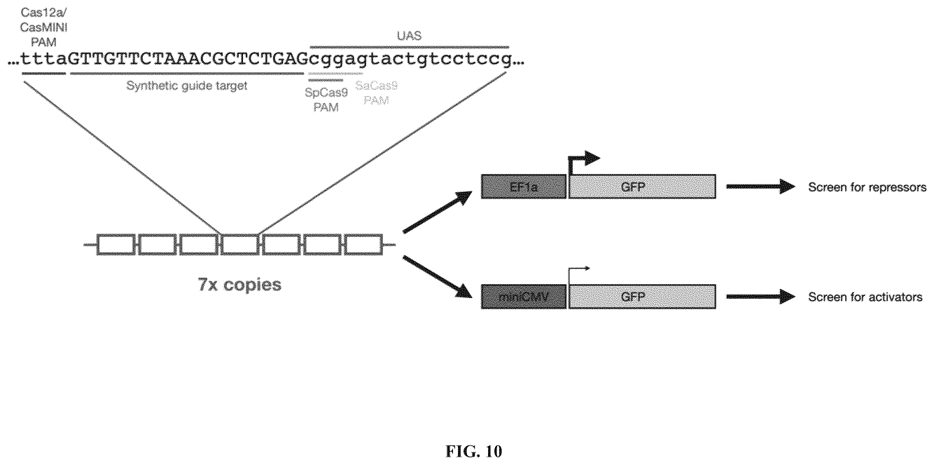

FIG. 10 schematically demonstrates an example structure of an expression vector for an ESR. The expression vector can comprise a plurality of copies (e.g., 7 copies) of a heterologous polynucleotide sequence. Each heterologous polynucleotide sequence can comprise a synthetic guide target (e.g., targetable by a guide RNA, such as SEQ ID NO: 49334), a plurality of CRISPR PAM sequences (e.g., Cas12a PAM sequence such as “TTTA”, SpCas9 PAM sequence such as “CGG”, SaCas9 PAM sequence such as “CGGAG”, and/or an Upstream Activating Sequence (UAS) sequence, and some of these components may overlap with each other (e.g., may share one or more common nucleobases, such as a common polynucleotide sequence). The UAS (e.g., upstream of a promoter) can be targeted by a transcription activator, and such targeting can be sufficient to regulate (e.g., enhance) expression of a target gene under the control of the promoter.

The synthetic guide target can comprise a polynucleotide sequence and an additional polynucleotide sequence from two different sources (e.g., a polynucleotide sequence (e.g., 10 nucleobase length) from a guide target of human CD45 (chr1) and an additional polynucleotide sequence (e.g., 10 nucleobase length) from a guide target of human CD71 (chr3)).

The UAS can be from yeast GAL4 promoter (e.g., bound by GAL4 protein in yeast). The UAS may not exhibit or affect any function in mammalian cells (e.g., human cells), thus minimizing side effects or off target effects of having the expression vector in the mammalian cells.

The plurality of copies of the heterologous polynucleotide sequence can be upstream of a gene, such as a target gene (e.g., a gene and its promoter sequence). The target gene can encode a protein naturally present in the cell. The target gene can encode a protein that can be naturally occurring in the cell. Alternatively, the target gene can encode a protein that is not naturally occurring in the cell (e.g., a reporter protein, for instance a fluorescent protein, such as green fluorescent protein (GFP)). The target gene can be under the control of a promoter, such as strong constitutive human promoter (EF1a) or a weak minimal viral promoter (e.g., miniCMV).

In some examples, a population of ESR cells comprising the expression vector can be treated with a library of different heterologous gene effectors and a guide nucleic acid sequence against the synthetic guide target, and change in the expression level of the target gene can be measured to screen for lead heterologous gene effectors (e.g., repressors for an expression vector comprising a strong constitutive human promoter, or activators for an expression vector comprising a weak minimal viral promoter).

FIG. 11 schematically shows an example sequence of the ESR. FIG. 12 schematically shows an example sequence of a control reporter vector. In some cases, the spacing between each synthetic guide target sequence in the ESR can be different from that in the control reporter vector. For example, the spacing between each synthetic guide target sequence in the ESR can be longer than that in the control reporter vector.

### Example 8: Generation of Reporter Cell Lines Comprising the ESR

Various cells of different types can be engineered to comprise the ESR (e.g., as demonstrated in Example 7), to generate various types of reporter cell lines, e.g., that can be used in screening for heterologous gene effector domain(s) and complex(es) thereof that exhibit desirable properties for modulating target gene expression.

K562 cells can be engineered with ESR comprising miniCMV-GFP (ESRI 11), to screen for novel gene activators. K562 cells can be engineered with ESR comprising EF1a-GFP (ESR211), to screen for gene repressors. 293T cells can be engineered with ESR comprising miniCMV-GFP (ESR121), for validation of gene activators. 293T cells can be engineered with ESR comprising EF1a-GFP (ESR221), for validation of gene repressors.

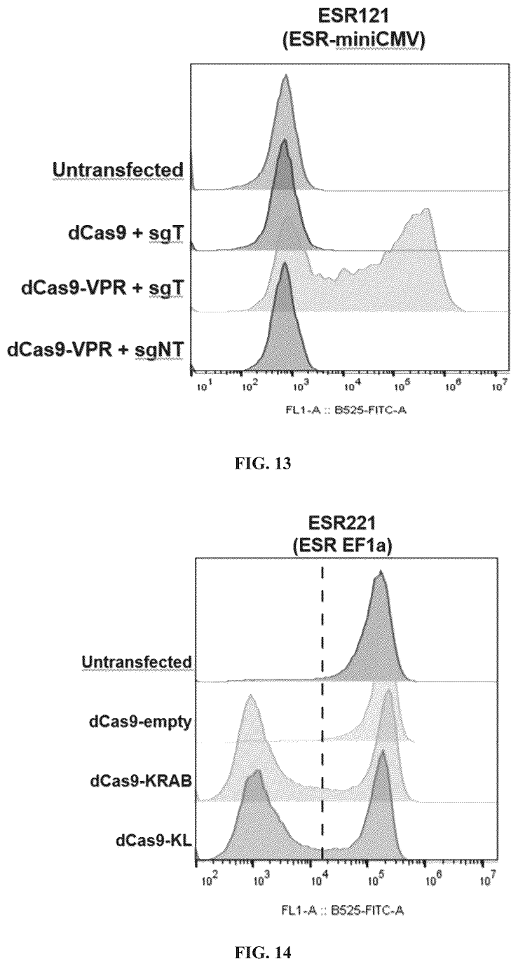

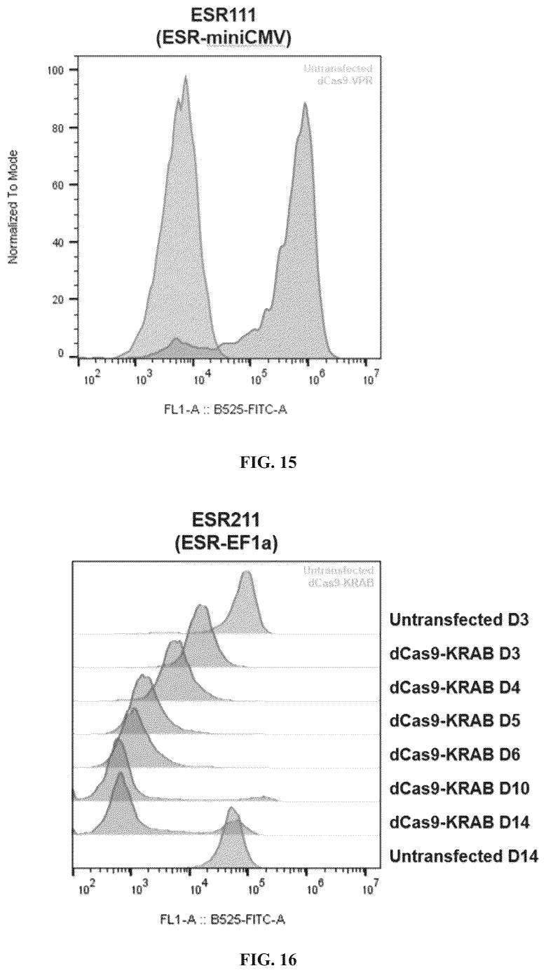

FIG. 13 shows flow cytometry data analysis of a reporter 293T cell line engineered with the ESR encoding miniCMV-GFP (ESR121), either (i) untransfected, (ii) transfected with dCas9 and a guide RNA against the ESR vector (dCas9+sgT), (iii) transfected with dCas9-VPR activator and a guide RNA against the ESR vector (dCas9-VPR+sgT), or (iv) transfected with dCas9-VPR activator and a non-targeting control guide RNA against the ESR vector (dCas9-VPR+sgNT). FIG. 14 shows flow cytometry data analysis of a reporter 293T cell line engineered with the ESR encoding EF1a-GFP (ESR221), either (i) untransfected, (ii) transfected with dCas9 and a guide RNA against the ESR vector (dCas9-empty), (iii) transfected with dCas9-KRAB repressor and a guide RNA against the ESR vector (dCas9-KRAB), or (iv) transfected with dCas9-KRAB-DNMT3L repressor and a guide RNA against the ESR vector (dCas9-KL). Based on FIG. 13 and FIG. 14, 293T ESR cells exhibit potent activation or repression following dCas9-VPR or dCas9-KRAB transfection.

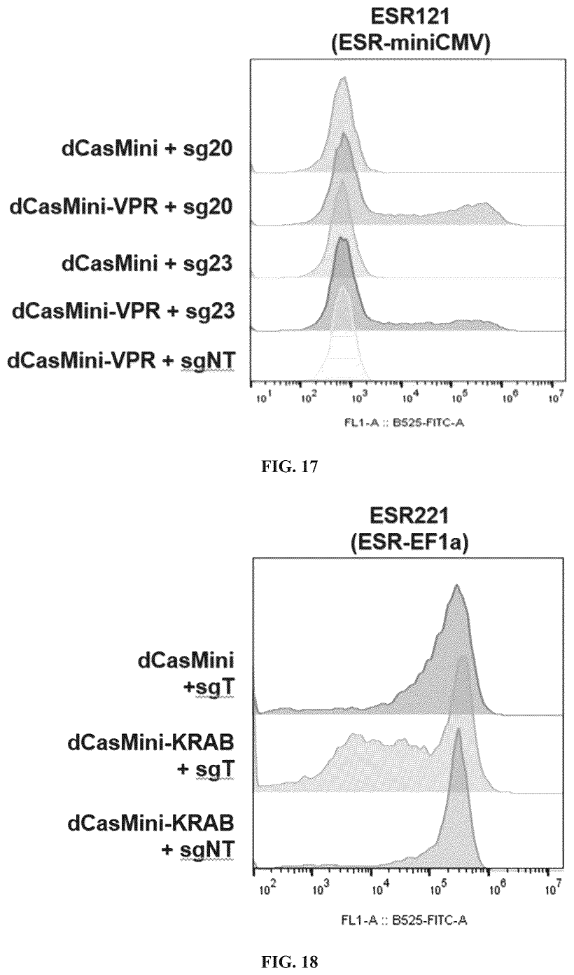

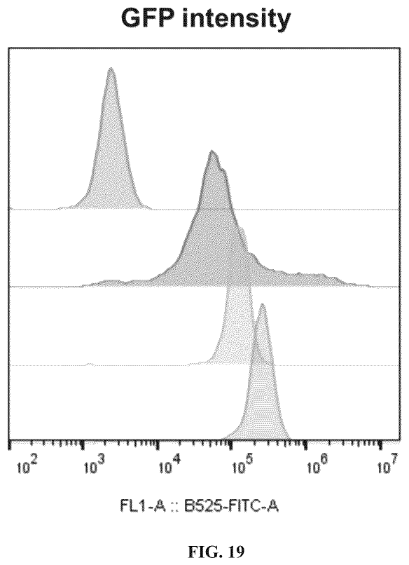

FIG. 15 shows flow cytometry data analysis of a reporter K562 cell line engineered with the ESR encoding miniCMV-GFP (ESR111), either (i) untransfected or (ii) transfected with dCas9-VPR and a guide RNA against the ESR vector (dCas9-VPR). FIG. 16 shows flow cytometry data analysis of a reporter K562 cell line engineered with the ESR encoding EF1a-GFP (ESR211), either untransfected and measured on day 3 (untransfected D3), untransfected and measured on day 14 (untransfected D14), transfected with dCas9-KRAB and a guide RNA against the ESR vector and measured on day 3 (dCas9-KRAB D3), transfected with dCas9-KRAB and a guide RNA against the ESR vector and measured on day 4 (dCas9-KRAB D4), transfected with dCas9-KRAB and a guide RNA against the ESR vector and measured on day 5 (dCas9-KRAB D5), transfected with dCas9-KRAB and a guide RNA against the ESR vector and measured on day 6 (dCas9-KRAB D6), transfected with dCas9-KRAB and a guide RNA against the ESR vector and measured on day 10 (dCas9-KRAB D10), or transfected with dCas9-KRAB and a guide RNA against the ESR vector and measured on day 14 (dCas9-KRAB D14). Based on FIG. 15 and FIG. 16, K562 ESR cells exhibit potent activation or repression following dCas9-VPR or dCas9-KRAB mRNA transfection.

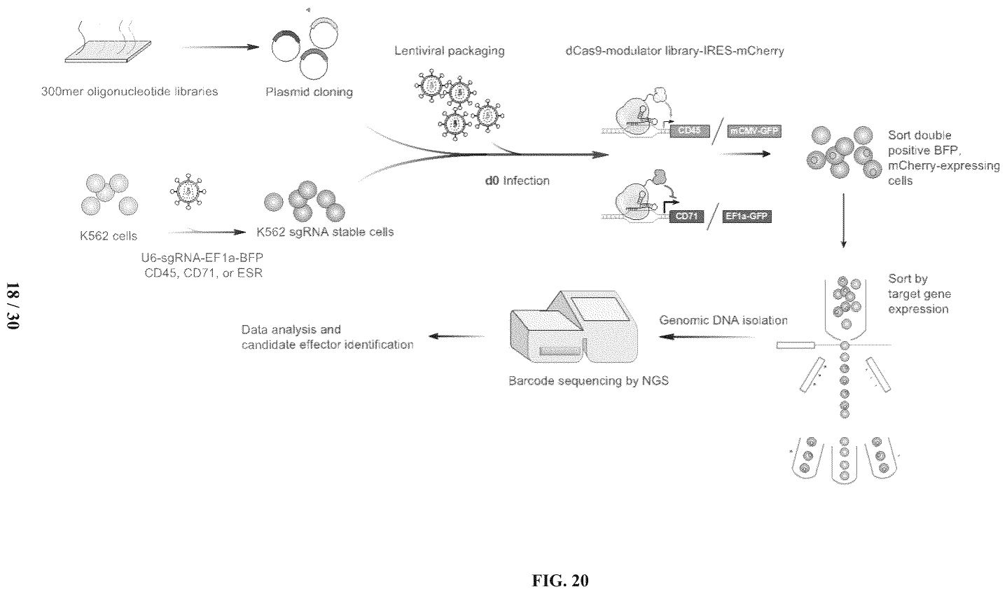

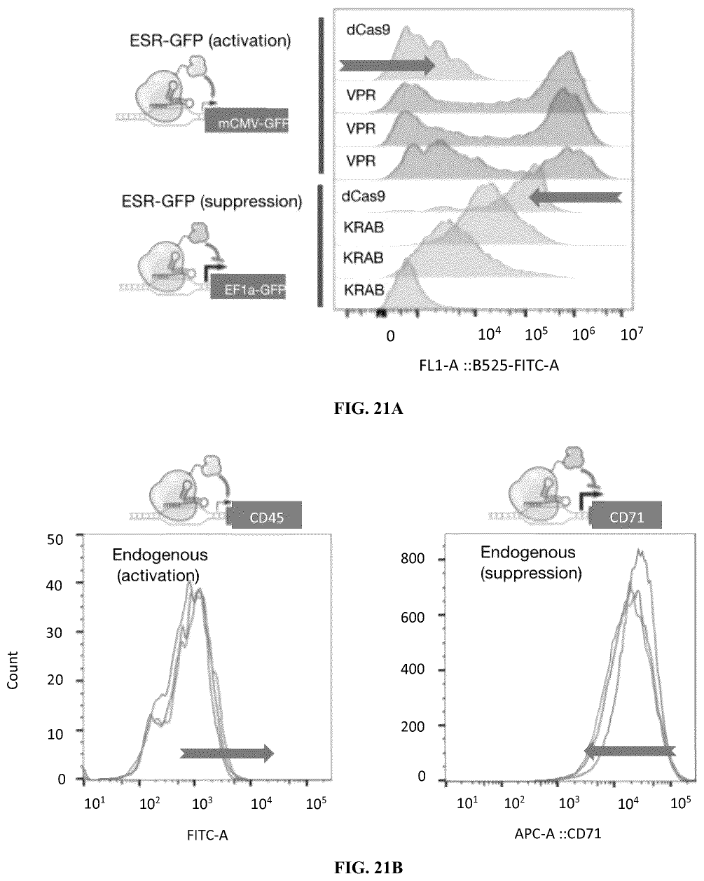

FIG. 17 shows flow cytometry data analysis of a reporter 293T cell line engineered with the ESR encoding miniCMV-GFP (ESR121), either (i) transfected with dCasMINI and a guide RNA against the ESR vector (dCasMINI+sg20), (ii) transfected with dCasMINI-VPR and the guide RNA against the ESR vector (dCasMINI-VPR+sg20), (iii) transfected with dCasMINI and a different guide RNA against the ESR vector (dCasMINI+sg23), (iv) transfected with dCasMINI-VPR and the different guide RNA against the ESR vector (dCasMINI-VPR+sg23), or (v) transfected with dCasMINI-VPR and a control non-specific guide RNA sequence (dCasMINI-VPR+sgNT). FIG. 18 shows flow cytometry data analysis of a reporter 293T cell line engineered with the ESR encoding EF1a-GFP (ESR221), either (i) transfected with dCasMINI and a guide RNA against the ESR vector (dCasMINI+sgT), (ii) transfected with dCasMINI-KRAB and a guide RNA against the ESR vector (dCasMINI-KRAB+sgT), or (iii) transfected with dCasMINI-KRAB and a control non-specific guide RNA sequence (dCasMINI-KRAB+sgNT). Based on FIG. 17 and FIG. 18, 293T ESR cells is strongly repressed by dCasMini-KRAB, but activation by dCasMINI-VPR is less robust.

### Example 9: Effector library screen

A custom DNA oligo library encoding>46,000 candidate heterologous gene effectors (each 255 bp), paired to unique 12mer DNA unique molecular identifiers (e.g., barcodes) and cloning homology arms, was cloned into dCas9 lentiviral expression plasmid. The candidate heterologous gene effectors encoded included each of SEQ ID NOs: 16-47350 and 49353-50052, which includes candidate heterologous gene effectors derived from human proteins (e.g., SEQ ID NOs: 16-13605, identified as described in Example 1), control effectors (e.g., SEQ ID NOs: 13606-16154), candidate heterologous gene effectors derived from viral proteins (e.g., SEQ ID NOs: 16155-43953, identified as described in examples 3 & 4), and additional control effectors (e.g., SEQ ID NOs: 43954-47350). Additional peptide sequences encoded downstream of putative stop codons within the 255 bp sequences are provided in SEQ ID NOs: 47351-49333.

In parallel, custom GFP reporters (engineered synthetic reporter (ESR)-GFP) were generated by placing 7 copies of a unique guide RNA targeting sequence (tttaGTTGTTCTAAACGCTCTGAGegg, SEQ ID NO: 49339; CasMini and Cas9 PAM sequences indicated in bold and underlined text, respectively) upstream of a minimal CMV promoter driving GFP to test for activators, or a constitutive human EF1a promoter driving GFP to test for repressors (e.g., as described in Example 7 and FIG. 10). These reporter plasmids were packaged into lentivirus and transduced into K562 cells. Following puromycin selection for transduced cells, individual cells were isolated by limiting dilution. After clonal expansion, several clones of each reporter cell line were tested by transfection of canonical modulators and favorable clones were selected for downstream experiments.

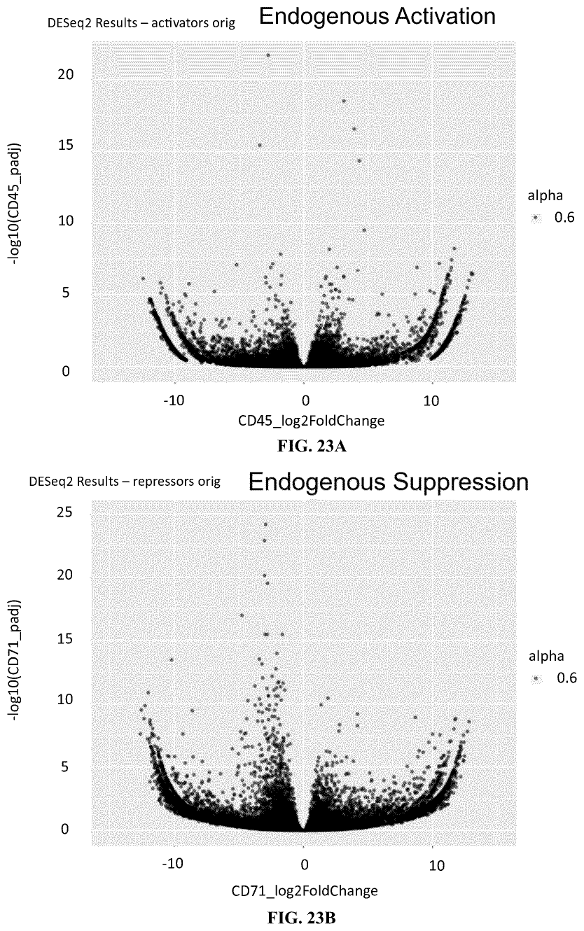

sgRNA-BFP expression cassettes targeting ESR-GFP, CD45, or CD71 were individually transduced into ESR-GFP or WT K562 cells followed by FACS for BFP+ cells. The dCas9-candidate effector library was packaged into lentivirus and delivered into K562 cells expressing the appropriate sgRNAs. Transduced cells were enriched by blasticidin selection, followed by fluorescence-activated cell sorting (FACSAria) to separate populations of interest (GFP-ON cells for activation and GFP-OFF cells for repression) at 10 days post-transduction. Sorted populations of interest were further enriched by culturing for 6 additional days and subjected to 4-way gated FACS separation into discrete bins based on GFP fluorescence intensity. Genomic DNA was extracted from each discrete bin, as well as bulk GFP-ON and GFP-OFF cells, then processed in parallel into next-generation sequencing (NGS) libraries and sequenced to identify barcodes present in each sample (Illumina Next-Seq, 1×75 bp). The workflow is summarized in FIG. 20.

### Screen Using Engineered Synthetic Reporter (ESR) System

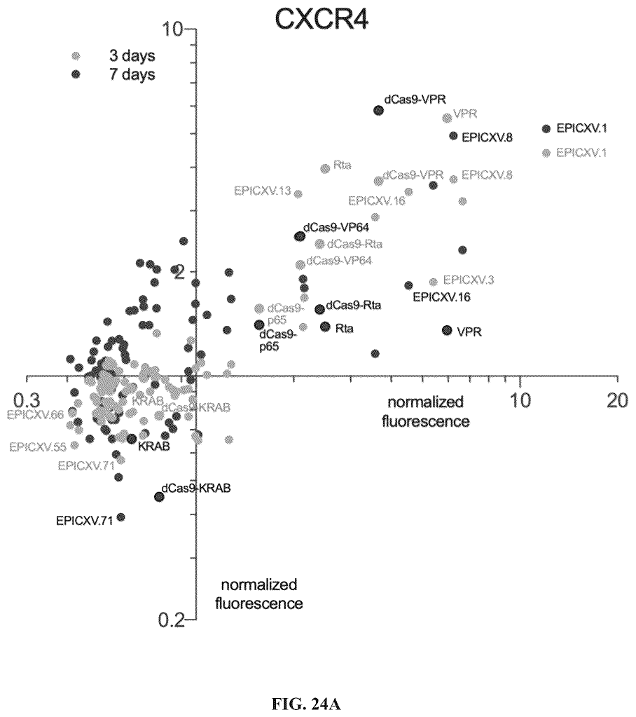

The library of candidate heterologous gene effectors was screened in ESR-GFP reporter K562 cells. The ESR-GFP reporters exhibited high dynamic range in response to activation with control dCas9-VPR and repression in response to control dCas9-KRAB (FIG. 21A).

Read count matrices for each library were generated based on alignment of sequenced barcodes corresponding to heterologous gene effectors. All subsequent data analysis was performed using R version 4.1.0.

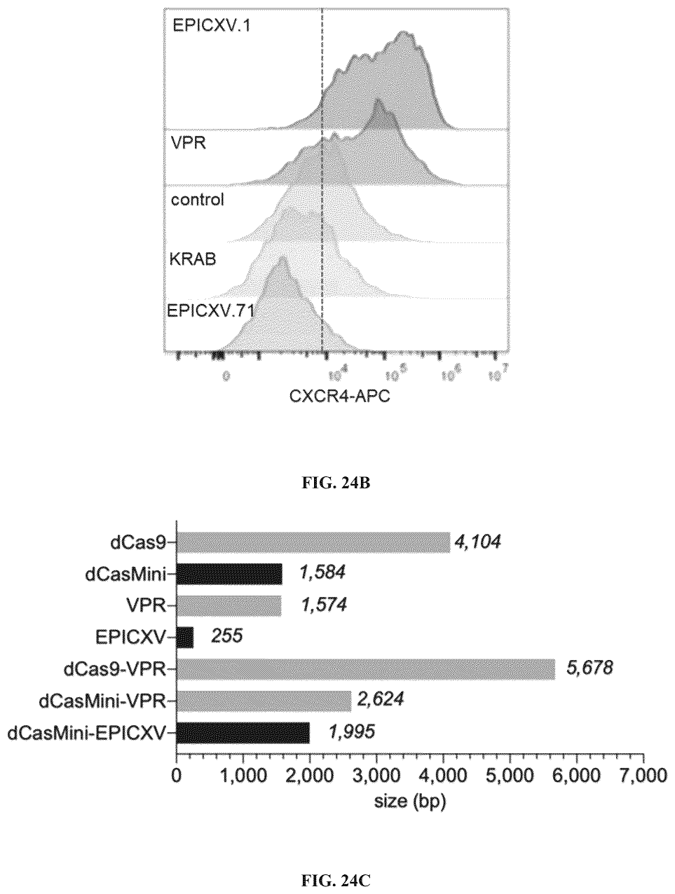

For the activation screen, technical replicates for GFP-OFF libraries were collapsed (counts per barcode were summed) resulting in two GFP-OFF replicates. For the GFP-ON conditions, one GFP-ON library was collected at 10 days post-transduction and another was built in silico by taking the weighted sum of all GFP-ON gates (collected after a further 6 days of enrichment). DESEQ2 (version 1.32.0) was used to identify statistically significant activator elements that were enriched in the two GFP-ON libraries compared to the two GFP-OFF libraries. A volcano plot showing hits in the activator screen is provided in FIG. 22A.

For the repressor screen, technical replicates for GFP-ON libraries were collapsed (counts per barcode were summed) resulting in two GFP-ON replicates. For the GFP-OFF conditions, one GFP-OFF was library collected at 10 days post-transduction and another that was built in silico by taking the weighted sum of all GFP-OFF gates (collected after a further 6 days of enrichment). DESEQ2 (version 1.32.0) was used to identify statistically significant suppressor elements that were enriched in the two GFP-OFF libraries compared to the two GFP-ON libraries. A volcano plot showing hits in the repressor screen is provided in FIG. 22B.

### Screen Using Endogenous Targets

The library of candidate heterologous gene effectors was screened in wild type K562 cells stably expressing sgRNAs against two endogenous human gene targets: lowly-expressed CD45 to screen for activation, or highly-expressed CD71 to screen for repression (FIG. 21B). Transduced cells were enriched by FACS, then stained with respective fluorophore-conjugated antibodies prior to 4-way gated FACS binning followed NGS library preparation and sequencing as above.

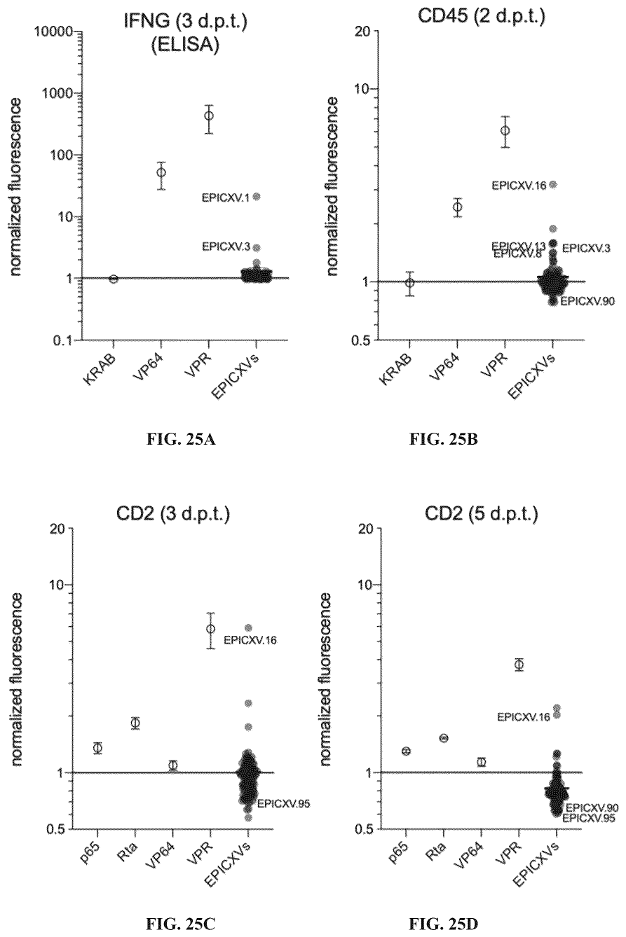

Read count matrices for each library were generated based on alignment of sequenced barcodes corresponding to heterologous gene effectors. All subsequent data analysis was performed using R version 4.1.0. For the activation screen (CD45), two replicates of the lowest GFP gate (GFP-OFF) were compared to two replicates of the highest GFP gate (GFP-ON) to identify novel activators. Any barcodes that had zero read counts in all conditions were removed, and then DESEQ2 (version 1.32.0) was used to identify statistically significant activator elements that were enriched in the two GFP-ON libraries compared to the two GFP-OFF libraries. A volcano plot showing hits in the activator screen is provided in FIG. 23A. The analysis for the repressor screen (CD71) was identical except that statistically significant repressor elements were identified that were enriched in the two GFP-OFF libraries compared to the two GFP-ON libraries. A volcano plot showing hits in the repressor screen is provided in FIG. 23B.

### Example 10: Arrayed-Format Heterologous Gene Effector Validations at an Endogenous Human Gene Locus

The top ˜200 candidate heterologous gene effectors identified in the screens of Example 9 ranked by false discovery rate (FDR) were individually re-synthesized and cloned into dCasMini expression plasmid for experimental validation by transient plasmid transfection in arrayed 96-well plate format. Wild-type HEK293T cells were seeded in 96-well plates at a density of 20,000 cells per well. 16 hours after seeding, cells were co-transfected with uniform masses of plasmids expressing sgRNA and candidate effector complex (effector-dCasMini fusion) in arrayed format such that each well received a different single candidate effector construct to be tested, but the same targeting sgRNA (sgCXCR4) across all wells. Each well received 100ng of effector-encoding plasmid and 50ng sgRNA plasmid, and experiments were performed in technical triplicate. Negative controls were dCas9 and dCasMini expression plasmids without any effector fusion, co-transfected with targeting and non-targeting sgRNA plasmids. Positive controls were dCas9 and dCasMini fusions to canonical transcriptional modulators: KRAB for repressor controls and p65, Rta, VP64, and VPR for activation controls.

At 3 and 7 days post-transfection (d.p.t.), cells were stained with CXCR4-APC conjugated antibody (BioLegend) and analyzed by flow cytometry (Cytoflex LX) to monitor CXCR4 protein expression, with analysis gates (FlowJo) to ensure measurements of live, singlet, and double-transfected cells to verify both effector and sgRNA plasmid expression via mCherry and BFP fluorescence, respectively. Geometric mean of APC fluorescence for each modulator was normalized against that of negative controls and reported as fold-change relative to negative controls.

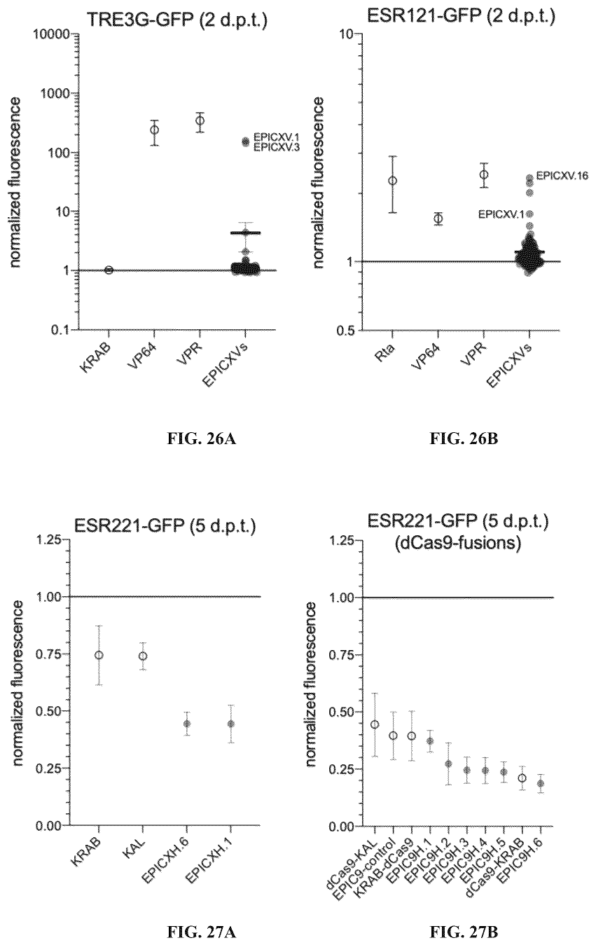

The impact of a number of candidate effectors on gene expression was shown to positively correlate between two independent experiments and between time points (FIG. 24A; the x-axis indicates normalized fluorescence at 3 d.p.t. (first of two independent experiments, each performed in triplicate), and the y-axis indicates normalized fluorescence measured in a second independent experiment (performed in triplicate). Data points represent dCasMini-modulator fusions unless indicated (dCas9). Candidate effectors are labelled with prefixes beginning with “EPIC” e.g., “EPICXV”, “EPIC9V”, “EPICXH”, or “EPIC9H” etc., followed by a numerical designation. Effectors were identified that resulted in upregulation of CXCR4 expression on day 3 and/or day 7 (e.g., EPICXV.1, SEQ ID NO: 23631; EPICXV.8, SEQ ID NO: 35737; EPICXV.3, SEQ ID NO: 23639); EPICXV.16, SEQ ID NO: 17629; or EPICXV.13, SEQ ID NO: 38138; or downregulation of CXCR4 expression on day 3 and/or day 7 (e.g., EPICXV.55, SEQ ID NO: 19860; EPICXV.66, SEQ ID NO: 40986; EPICXV.71, SEQ ID NO: 33890). These candidate effectors altered expression of the target gene to comparable or superior degree compared to control activators or suppressors (FIG. 24B, representative flow cytometry histograms at 3 d.p.t. for fusions of candidate heterologous gene effectors to dCasMini). The candidate effectors induced the observed changes in target gene expression despite being considerably smaller than the control effectors (FIG. 24C).

### Example 11: Gene Expression Modulation at Endogenous Loci

This example demonstrates the effect of candidate heterologous gene effectors fused to dCasMini on expression of endogenous target genes.

Wildtype HEK293T cells were seeded and transiently transfected as in Example 10 with plasmids expressing candidate effector-dCasMini fusion and sgRNA targeting promoters of human IFNG, CD45, or CD2.

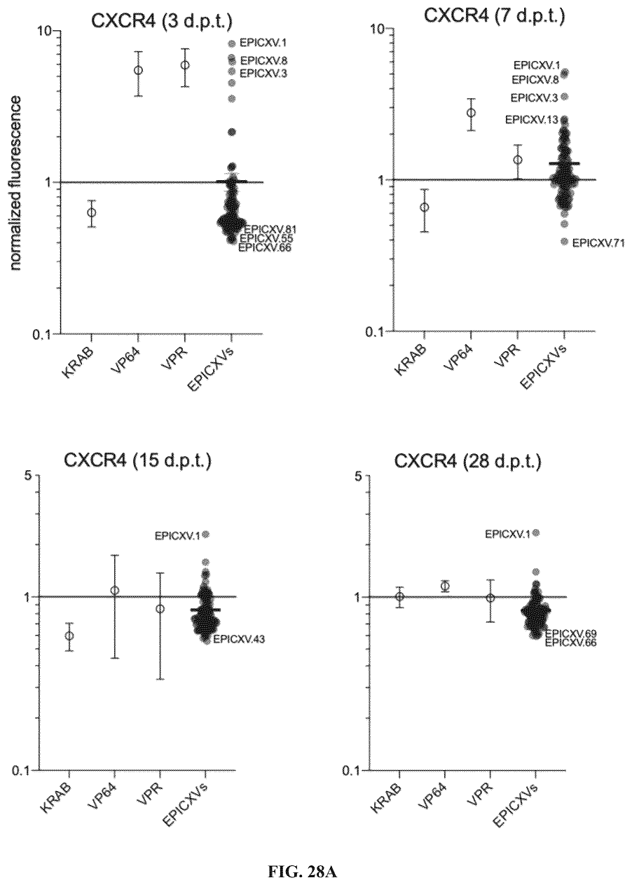

At 72 hours post-transfection, supernatants of cells transfected with sgRNA targeting IFNG were collected to monitor IFNG protein expression by ELISA, after verifying both effector and sgRNA plasmid expression via mCherry and BFP fluorescence, respectively, by fluorescence microscopy (EVOS FL). Interferon gamma ELISA protein concentration for each effector was normalized against that of negative controls and reported as fold-change relative to negative controls. A range of effects on interferon gamma production was observed (FIG. 25A).

At 2 days post-transfection, cells transfected with sgRNA targeting CD45 were stained with APC-conjugated anti-CD45 antibody (BioLegend) and analyzed by flow cytometry (Cytoflex LX), with analysis gates (FlowJo) to ensure measurements of live, singlet, and double-transfected cells to verify both effector and sgRNA plasmid expression via mCherry and BFP fluorescence, respectively. Geometric mean of APC fluorescence for each effector was normalized against that of negative controls and reported as fold-change relative to negative controls. A range of effects on CD45 expression was observed (FIG. 25B).

At 3 and 5 days post-transfection with sgRNA targeting CD2, were stained with APC-conjugated anti-CD2 antibody (BioLegend) and analyzed by flow cytometry (Cytoflex LX), with analysis gates (FlowJo) to ensure measurements of live, singlet, and double-transfected cells to verify both effector and sgRNA plasmid expression via mCherry and BFP fluorescence, respectively. Geometric mean of APC fluorescence for each effector was normalized against that of negative controls and reported as fold-change relative to negative controls. A range of effects on CD2 expression was observed (FIGS. 25C and 25D).

### Example 12: Gene Expression Modulation in Reporter Cells

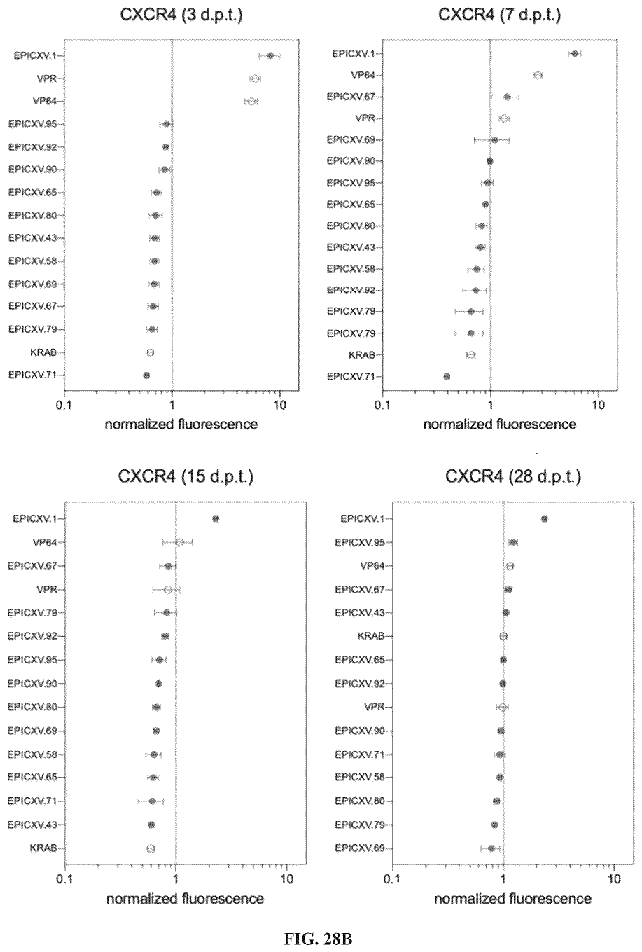

HEK293T cells bearing a stably integrated TRE3G promoter-driven GFP reporter were seeded and transiently transfected as in Example 10 with plasmids expressing candidate effector-dCasMini fusions and sgRNA targeting the synthetic TET promoter. 48 hours post-transfection, cells were analyzed by flow cytometry (Cytoflex LX) to monitor GFP expression, with analysis gates (FlowJo) to ensure measurements of live, singlet, and double-transfected cells to verify both effector and sgRNA plasmid expression via mCherry and BFP fluorescence, respectively. Geometric mean of GFP fluorescence for each effector was normalized against that of negative controls and reported as fold-change relative to negative controls. A range of effects on reporter expression was observed (FIG. 26A).

HEK293T cells bearing a stably integrated GFP synthetic reporter driven by low-expression miniCMV promoter (cell line ESR121) were seeded, transiently transfected as in Example 10 with plasmids expressing candidate effector-dCasMini fusions and sgRNA targeting the reporter, and analyzed as above 2 days post-transfection to measure transcriptional activation by dCasMini-effector fusions. A range of effects on reporter expression was observed (FIG. 26B).

### Example 13: Repression of Gene Expression in Reporter Cells

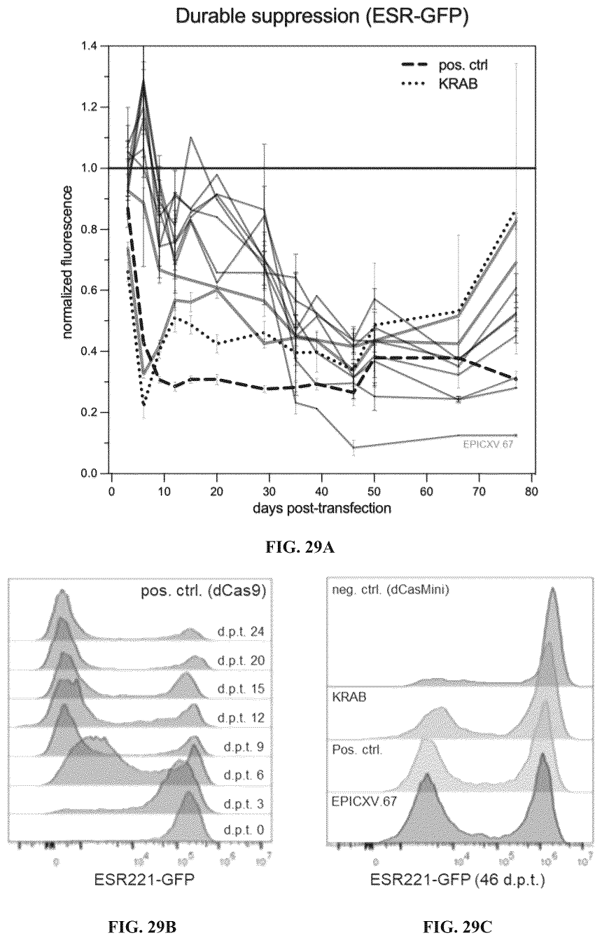

Effectors with repression activity identified from the library of candidate effectors derived from human peptide sequences were tested individually as fusions to dCasMini. HEK293T cells bearing a stably integrated GFP synthetic reporter driven by high-expression EFlu promoter (cell line ESR221) were seeded, transiently transfected as in Example 10 with plasmids expressing candidate effector-dCasMini or —dCas9 fusions and sgRNA targeting the reporter, and analyzed at 5 days post-transfection to measure transcriptional repression by dCasMini-effector fusions. Illustrative candidate effector domains EPICXH.6 (SEQ ID NO: 9066) and EPICXH.1 (SEQ ID NO: 1102) showed strong repression of reporter expression (FIG. 27A).
Additional candidate human repressor domains were tested as fusions to dCas9. HEK293T cells bearing a stably integrated GFP synthetic reporter driven by high-expression EFlu promoter (cell line ESR221) were seeded, transiently transfected, and analyzed as above at 5 days post-transfection to measure transcriptional suppression. Illustrative effector domains EPIC9H.1 (SEQ ID NO: 1102), EPIC9H.2 (SEQ ID NO: 5543), EPIC9H.3 (SEQ ID NO: 2057), EPIC9H.4 (SEQ ID NO: 15646), EPIC9H.5 (SEQ ID NO: 11948), and EPIC9H.6 (SEQ ID NO: 9066) showed strong repression of reporter expression (FIG. 27B).

### Example 14: Durability of Transcriptional Effects at Endogenous CXCR4 Locus

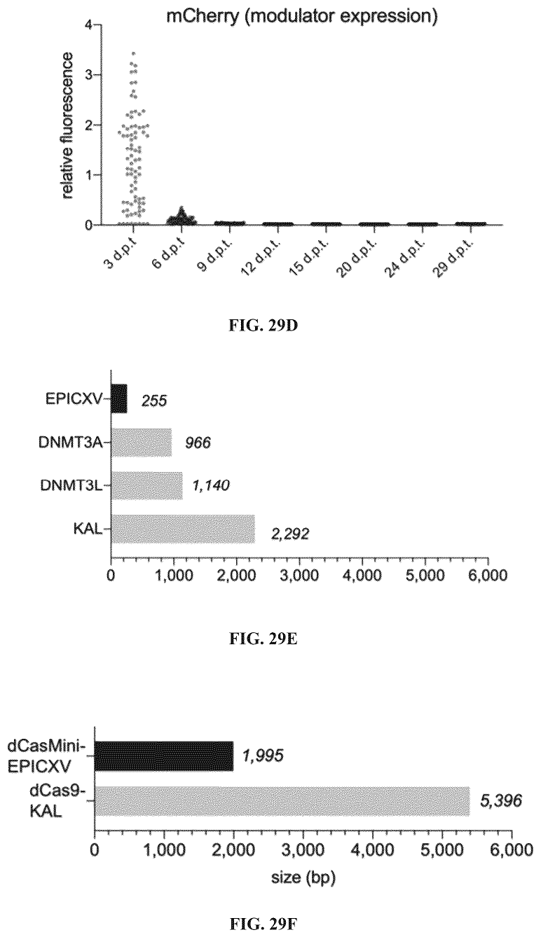

Wild-type HEK293T cells were transfected with plasmids expressing sgRNA targeting CXCR4 and candidate effector complex (effector-dCasMini fusion). Transfected cells were serially analyzed for transcriptional modulation of CXCR4 as in Example 10 on day 3, 7, 15, and 28 post-transfection (FIG. 28A, control effectors KRAB, VP64, and VPR, are depicted as clear circles and novel effectors are shown as dark circles representing the mean of replicates for each individual modulator). Normalized fluorescence values for selected individual novel modulators relative to positive controls at each time point are shown in FIG. 28B, with modulators ranked by effect size at each time point. Candidate heterologous gene effectors showing interesting activity included EPICXV.1 (SEQ ID NO: 23631), EPICXV.95 (SEQ ID NO: 40913), EPICXV.92 (SEQ ID NO: 22707), EPICXV.90 (SEQ ID NO: 42623), EPICXV.65 (SEQ ID NO: 22149), EPICXV.80 (SEQ ID NO: 25430), EPICXV.43 (SEQ ID NO: 34047), EPICXV.58 (SEQ ID NO: 21166), EPICXV.69 (SEQ ID NO: 25555), EPICXV.67 (SEQ ID NO: 40985), EPICXV.79 (SEQ ID NO: 38780), and EPICXV.71 (SEQ ID NO: 33890). A number of candidate effectors exhibited interesting activity, including strong upregulation of CXCR4 expression by EPICXV.1, which was observed at every time point.

### Example 15: Durability of Transcriptional Effects

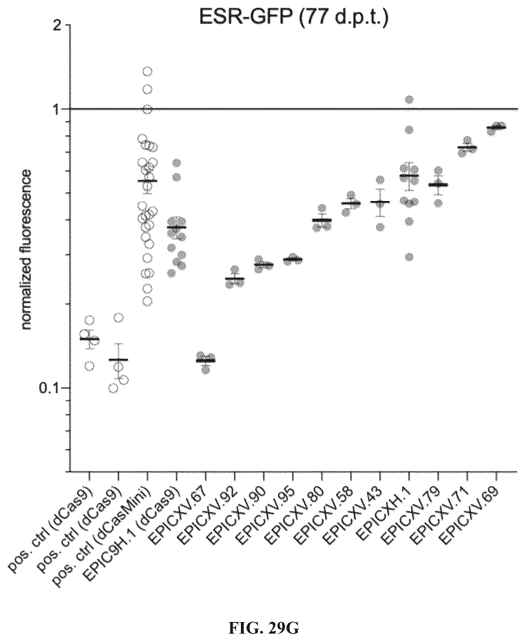

HEK293T cells bearing a stably integrated GFP synthetic reporter driven by high-expression EFlu promoter were seeded, transiently transfected, with plasmids expressing candidate effector-dCasMini fusions and sgRNA targeting the reporter, and analyzed serially up to 77 days post-transfection. Durable repression of gene expression was observed for several candidate heterologous gene effectors, including EPICXV.67 (SEQ ID NO: 40985) (FIG. 29A, positive control suppression effectors are indicated as dashed lines, while novel modulators are solid lines.

Reporter gene expression is shown over time in FIG. 29B for a positive control dCas9-KAL (a construct for persistent suppression comprising KRAB, DNMT3A, and DNMT3L domains fused to dCas9), demonstrating increased repression of GFP over progressive time points. Representative histograms for a negative control, positive controls, and a candidate heterologous gene effector (EPICXV.97) are provided in FIG. 29C, showing the candidate effector shows a strong and durable effect on gene expression.

Longitudinal measurements of expression of an mCherry reporter, indicative of expression of the heterologous gene effector, showed that expression of the effector drops precipitously after 6-9 d.p.t. and remains undetectable throughout subsequent time points, suggesting that any transcriptional effects observed beyond 9 d.p.t. are not attributable to maintained expression of the effector (FIG. 29D). The strong and durable effects on gene expression were observed despite the small size of the effector domain (encoded by a 255 nucleotide coding sequence) as compared to domains in control effectors (FIG. 29E) and fusion proteins (FIG. 29F).

Data collected on day 77 post-transfection showed that a number of candidate heterologous gene effectors showed varying degrees of repression of target gene expression that endured to this time point (FIG. 29G). Effectors exhibiting durable effects on expression included EPICXV.67 (SEQ ID NO: 40985), EPICXV.92 (SEQ ID NO: 22707), EPICXV.90 (SEQ ID NO: 42623), EPICXV.95 (SEQ ID NO: 40913), EPICXV.80 (SEQ ID NO: 25430), EPICXV.58 (SEQ ID NO: 21166), EPICXV.43 (SEQ ID NO: 34047), EPICXH.1 (SEQ ID NO: 1102), EPICXV.79 (SEQ ID NO: 38780), EPICXV.71 (SEQ ID NO: 33890), and EPICXV.69 (SEQ ID NO: 25555).

### Example 16: Comparison of Endogenous and Synthetic Reporter Data

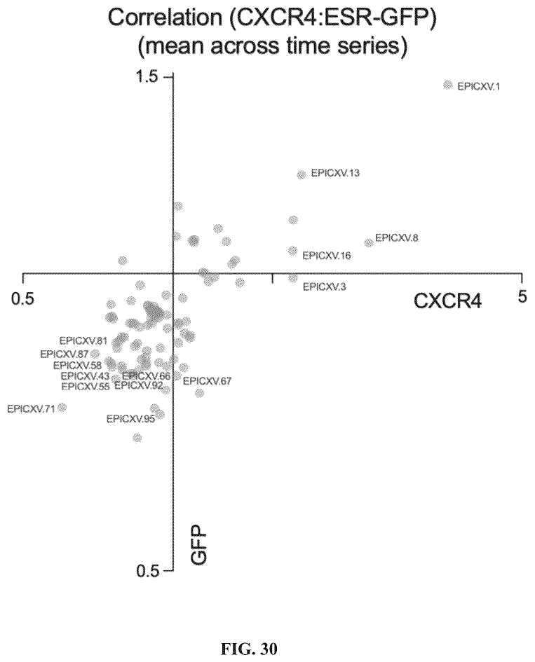

Data collected for heterologous gene effectors was compared for effects on expression of endogenous gene CXCR4 and ESR-GFP synthetic reporter cells (FIG. 30; identified effectors include EPICXV.81 (SEQ ID NO: 21015), EPICXV.87 (SEQ ID NO: 32678), and other effectors described herein). A correlation can be observed between normalized GFP fluorescence in ESR-GFP synthetic reporter cells (averaged across all time points from 3 to 77 d.p.t.; y-axis) and normalized CXCR4-APC fluorescence (averaged across all time points from 3 to 28 d.p.t.; x-axis).

1. (canceled)

2. A method, comprising:
(a) contacting a population of cells with a library of complexes, wherein an individual complex of the library comprises:
(i) a heterologous gene effector that is different from heterologous gene effectors in other complexes of the library; and
(ii) a guide nucleic acid sequence that exhibits 100% sequence identity to guide nucleic acid sequences in the other complexes of the library,
wherein the heterologous gene effector and the guide nucleic acid sequence form the individual complex that exhibits specific binding to a target endogenous gene in the population of cells, and
wherein the heterologous gene effector comprises a viral gene effector;
(b) upon the contacting, sorting the population of cells based on a change in expression or activity level of the target endogenous gene in the population of cells; and
(c) identifying one or more lead heterologous gene effectors of the library that effect the change.

3-51. (canceled)

52. A complex comprising a guide moiety and a heterologous gene effector, wherein the heterologous gene effector comprises an amino acid sequence with at least about 70% sequence identity to any one of SEQ ID NOs: 23631, 1102, 2057, 5543, 9066, 11948, 15646, 17629, 19860, 21015, 21166, 22149, 22707, 23639, 25430, 25555, 32678, 33890, 34047, 35737, 38138, 38780, 40913, 40985, 40986, or 42623.

53. (canceled)

54. The complex of claim 52, wherein the heterologous gene effector comprises the amino acid sequence of any one of SEQ ID NOs: 23631, 1102, 2057, 5543, 9066, 11948, 15646, 17629, 19860, 21015, 21166, 22149, 22707, 23639, 25430, 25555, 32678, 33890, 34047, 35737, 38138, 38780, 40913, 40985, 40986, or 42623.

55. The complex of claim 52, wherein the amino acid sequence has at least 90% sequence identity to SEQ ID NO: 23631.

56. (canceled)

57. The complex of claim 52, wherein the amino acid sequence has at least 90% sequence identity to SEQ ID NO: 33890.

58. (canceled)

59. The complex of claim 52, wherein the amino acid sequence has at least 90% sequence identity to SEQ ID NO: 40985.

60. (canceled)

61. The complex of claim 52, wherein the heterologous gene effector contains less than 500 amino acids.

62. (canceled)

63. (canceled)

64. The complex of claim 52, wherein the guide moiety comprises a guide nucleic acid sequence.

65. The complex of claim 64, wherein the guide nucleic acid sequence comprises between about 10 and about 30 nucleotides.

66. The complex of claim 64, wherein the guide nucleic acid sequence is a guide RNA.

67. (canceled)

68. The complex of claim 52, wherein the guide moiety comprises a nuclease or a part thereof.

69-71. (canceled)

72. The complex of claim 68, wherein the nuclease or part thereof is a nuclease deactivated Cas (dCas) protein or part thereof.

73. The complex of claim 52, the guide moiety and the heterologous gene effector are fused to each other.

74. The complex of claim 52, wherein the guide moiety and the heterologous gene effector are non-covalently coupled to each other.

75. (canceled)

76. (canceled)

77. A vector comprising the complex of claim 52.

78. A vector comprising a nucleic acid that encodes the heterologous gene effector of claim 52.

79. The vector of claim 78, wherein the vector further comprises a nucleic acid that encodes the guide moiety or a component thereof.

80. (canceled)

81. (canceled)

82. A population of cells comprising the complex of claim 52.

83. A method of modulating expression or activity of a target gene, the method comprising contacting a population of cells that comprise the target gene with the complex of claim 52.

84-89. (canceled)

90. A method of treating a subject in need thereof, the method comprising administering to the subject the complex of claim 52, thereby modulating expression or activity of a target gene in a population of cells in the subject.

91-103. (canceled)

104. An expression vector comprising: a plurality of heterologous polynucleotide sequences, wherein each heterologous polynucleotide sequence of said plurality of heterologous polynucleotide sequences exhibits at least about 80% sequence identity to the polynucleotide sequence of any one of SEQ ID NOs: 49334-49338.

105. A nucleic acid molecule comprising:
a heterologous polynucleotide sequence is a chimeric sequence comprising (i) a CRISPR target sequence and (ii) a CRISPR protospacer adjacent motif (PAM) sequence and an additional CRISPR PAM sequence that are different,
wherein said CRISPR target sequence is flanked by said CRISPR PAM sequence and said additional CRISPR PAM sequence.

106. A nucleic acid molecule comprising:
a plurality of heterologous polynucleotide sequences, wherein:
(i) each heterologous polynucleotide sequence of said plurality of heterologous polynucleotide sequences comprises a polynucleotide sequence and an additional polynucleotide sequence that are derived from different human chromosomes; and
(ii) a size of said each heterologous polynucleotide sequence is at most about 50 nucleobases.
